SEQUENCE LISTING (206 sequences)

SEQ ID NO: 1 - protein, 5 aa, Bacteroides fragilis
SDDDD

SEQ ID NO: 2 - protein, 5 aa, Flavobacterium johnsoniae
SDDFE

SEQ ID NO: 3 - protein, 5 aa, Artificial Sequence
  Mutated sequence of SEQ ID 1
SEEEE

SEQ ID NO: 4 - protein, 5 aa, Artificial Sequence
  Mutated sequence of SEQ ID 1
SDEDE

SEQ ID NO: 5 - protein, 5 aa, Artificial Sequence
  Mutated sequence of SEQ ID 1
SEDED

SEQ ID NO: 6 - protein, 5 aa, Artificial Sequence
  Mutated sequence of SEQ ID 1
SDDEE

SEQ ID NO: 7 - protein, 5 aa, Artificial Sequence
  Mutated sequence of SEQ ID 1
SEEDD

SEQ ID NO: 8 - protein, 5 aa, Artificial Sequence
  Mutated sequence of SEQ ID 1
SEEED

SEQ ID NO: 9 - protein, 5 aa, Artificial Sequence
  Mutated sequence of SEQ ID 1
SDDDE

SEQ ID NO: 10 - protein, 5 aa, Artificial Sequence
  Mutated sequence of SEQ ID 2
SEEFE

SEQ ID NO: 11 - protein, 5 aa, Artificial Sequence
  Mutated sequence of SEQ ID 2
SEEFD

SEQ ID NO: 12 - protein, 5 aa, Artificial Sequence
  Mutated sequence of SEQ ID 2
SEDFE

SEQ ID NO: 13 - protein, 5 aa, Artificial Sequence
  Mutated sequence of SEQ ID 2
SDEFE

SEQ ID NO: 14 - protein, 5 aa, Artificial Sequence
  Mutated sequence of SEQ ID 2
SEDFD

SEQ ID NO: 15 - protein, 5 aa, Artificial Sequence
  Mutated sequence of SEQ ID 2
SDEFD

SEQ ID NO: 16 - protein, 5 aa, Capnocytophaga canimorsus
QKDDE

SEQ ID NO: 17 - protein, 5 aa, Artificial Sequence
  Mutated sequence of SEQ ID 16
AKDDE

SEQ ID NO: 18 - protein, 5 aa, Artificial Sequence
  Mutated sequence of SEQ ID 16
AKDDA

SEQ ID NO: 19 - protein, 5 aa, Artificial Sequence
  Mutated sequence of SEQ ID 16
AKEEA

SEQ ID NO: 20 - protein, 5 aa, Artificial Sequence
  Mutated sequence of SEQ ID 16
QADDE

SEQ ID NO: 21 - protein, 6 aa, Artificial Sequence
  LES consensus sequence
  MISC_FEATURE (2)..(2): any amino acid
  MISC_FEATURE (6)..(6): any amino acid
CXKDEX

SEQ ID NO: 22 - protein, 6 aa, Artificial Sequence
  LES consensus sequence
  MISC_FEATURE (2)..(2): any amino acid
  MISC_FEATURE (6)..(6): any amino acid
CXKEDX

SEQ ID NO: 23 - protein, 6 aa, Artificial Sequence
  LES consensus sequence
  MISC_FEATURE (2)..(2): any amino acid
  MISC_FEATURE (6)..(6): any amino acid
CXKDDX

SEQ ID NO: 24 - protein, 6 aa, Artificial Sequence
  LES consensus sequence
  MISC_FEATURE (2)..(2): any amino acid
  MISC_FEATURE (6)..(6): any amino acid
CXKEEX

SEQ ID NO: 25 - protein, 5 aa, Artificial Sequence
  Mutated sequence of SEQ ID 1
TDDDD

SEQ ID NO: 26 - protein, 5 aa, Artificial Sequence
  Mutated sequence of SEQ ID 2
TDDFE

SEQ ID NO: 27 - protein, 5 aa, Artificial Sequence
  Mutated sequence of SEQ ID 1
TEEEE

SEQ ID NO: 28 - protein, 5 aa, Artificial Sequence
  Mutated sequence of SEQ ID 1
TDEDE

SEQ ID NO: 29 - protein, 5 aa, Artificial Sequence
  Mutated sequence of SEQ ID 1
TEDED

SEQ ID NO: 30 - protein, 5 aa, Artificial Sequence
  Mutated sequence of SEQ ID 1
TDDEE

SEQ ID NO: 31 - protein, 5 aa, Artificial Sequence
  Mutated sequence of SEQ ID 1
TEEDD

SEQ ID NO: 32 - protein, 5 aa, Artificial Sequence
  Mutated sequence of SEQ ID 1
TEEED

SEQ ID NO: 33 - protein, 5 aa, Artificial Sequence
  Mutated sequence of SEQ ID 1
TDDDE

SEQ ID NO: 34 - protein, 5 aa, Artificial Sequence
  Mutated sequence of SEQ ID 2
TEEFE

SEQ ID NO: 35 - protein, 5 aa, Artificial Sequence
  Mutated sequence of SEQ ID 2
TEEFD

SEQ ID NO: 36 - protein, 5 aa, Artificial Sequence
  Mutated sequence of SEQ ID 2
TEDFE

SEQ ID NO: 37 - protein, 5 aa, Artificial Sequence
  Mutated sequence of SEQ ID 2
TDEFE

SEQ ID NO: 38 - protein, 5 aa, Artificial Sequence
  Mutated sequence of SEQ ID 2
TEDFD

SEQ ID NO: 39 - protein, 5 aa, Artificial Sequence
  Mutated sequence of SEQ ID 2
TDEFD

SEQ ID NO: 40 - protein, 5 aa, Artificial Sequence
  LES consensus sequence
  MISC_FEATURE (1)..(1): any amino acid
XKDDD

SEQ ID NO: 41 - protein, 5 aa, Artificial Sequence
  LES consensus sequence
  MISC_FEATURE (1)..(1): any amino acid
XKEEE

SEQ ID NO: 42 - protein, 5 aa, Artificial Sequence
  LES consensus sequence
  MISC_FEATURE (1)..(1): any amino acid
XKDDE

SEQ ID NO: 43 - protein, 5 aa, Artificial Sequence
  LES consensus sequence
  MISC_FEATURE (1)..(1): any amino acid
XKDED

SEQ ID NO: 44 - protein, 5 aa, Artificial Sequence
  LES consensus sequence
  MISC_FEATURE (1)..(1): any amino acid
XKDEE

SEQ ID NO: 45 - protein, 5 aa, Artificial Sequence
  LES consensus sequence
  MISC_FEATURE (1)..(1): any amino acid
XKEDE

SEQ ID NO: 46 - protein, 5 aa, Artificial Sequence
  LES consensus sequence
  MISC_FEATURE (1)..(1): any amino acid
XKEDD

SEQ ID NO: 47 - protein, 5 aa, Artificial Sequence
  LES consensus sequence
  MISC_FEATURE (1)..(1): any amino acid
XKEED

SEQ ID NO: 48 - protein, 10 aa, Capnocytophaga canimorsus
CKKEVEVEEE

SEQ ID NO: 49 - protein, 7 aa, Capnocytophaga canimorsus
KKEVEEE

SEQ ID NO: 50 - protein, 7 aa, Artificial Sequence
  Mutated sequence of SEQ ID 49
AKEVEEE

SEQ ID NO: 51 - protein, 7 aa, Artificial Sequence
  Mutated sequence of SEQ ID 49
KKEAEEE

SEQ ID NO: 52 - protein, 7 aa, Artificial Sequence
  Mutated sequence of SEQ ID 49
KKEVEAE

SEQ ID NO: 53 - protein, 7 aa, Artificial Sequence
  Mutated sequence of SEQ ID 49
KAEVEEE

SEQ ID NO: 54 - protein, 7 aa, Artificial Sequence
  Mutated sequence of SEQ ID 49
KKAVEEE

SEQ ID NO: 55 - protein, 7 aa, Artificial Sequence
  Mutated sequence of SEQ ID 49
KKEVAEE

SEQ ID NO: 56 - protein, 7 aa, Artificial Sequence
  Mutated sequence of SEQ ID 49
AAEVEEE

SEQ ID NO: 57 - protein, 7 aa, Artificial Sequence
  Mutated sequence of SEQ ID 49
KKAAAAA

SEQ ID NO: 58 - protein, 7 aa, Artificial Sequence
  Mutated sequence of SEQ ID 49
KKAAAEE

SEQ ID NO: 59 - protein, 7 aa, Artificial Sequence
  Mutated sequence of SEQ ID 49
KKEVAAA

SEQ ID NO: 60 - protein, 7 aa, Artificial Sequence
  Mutated sequence of SEQ ID 49
RREVEEE

SEQ ID NO: 61 - protein, 7 aa, Artificial Sequence
  Mutated sequence of SEQ ID 49
RAEVEEE

SEQ ID NO: 62 - protein, 7 aa, Artificial Sequence
  Mutated sequence of SEQ ID 49
AREVEEE

SEQ ID NO: 63 - protein, 6 aa, Capnocytophaga canimorsus
KKEVEE

SEQ ID NO: 64 - protein, 5 aa, Capnocytophaga canimorsus
KKEVE

SEQ ID NO: 65 - protein, 5 aa, Artificial Sequence
  Mutated sequence of SEQ ID 64
KAEVE

SEQ ID NO: 66 - protein, 5 aa, Artificial Sequence
  Mutated sequence of SEQ ID 64
AKEVE

SEQ ID NO: 67 - protein, 5 aa, Artificial Sequence
  Mutated sequence of SEQ ID 64
KEVEE

SEQ ID NO: 68 - protein, 4 aa, Artificial Sequence
  LES consensus sequence
  MISC_FEATURE (1)..(1): X can be any amino acid
  MISC_FEATURE (2)..(2): X is selected from K, S, T and A with the proviso that when X(2) is A, X(1) is Q
XXDD

SEQ ID NO: 69 - protein, 4 aa, Artificial Sequence
  LES consensus sequence
  MISC_FEATURE (1)..(1): X can be any amino acid
  MISC_FEATURE (2)..(2): X is selected from K, S, T and A, with the proviso that when X(2) is A, X(1) is Q
XXDE

SEQ ID NO: 70 - protein, 4 aa, Artificial Sequence
  LES consensus sequence
  MISC_FEATURE (1)..(1): X can be any amino acid
  MISC_FEATURE (2)..(2): X is selected from K, S, T and A, with the proviso that when X(2) is A, X(1) is Q
XXED

SEQ ID NO: 71 - protein, 4 aa, Artificial Sequence
  LES consensus sequence
  MISC_FEATURE (1)..(1): X can be any amino acid
  MISC_FEATURE (2)..(2): X is selected from K, S, T and A, with the proviso that when X(2) is A, X(1) is Q
XXEE

SEQ ID NO: 72 - DNA, 30 bp, Artificial Sequence
  primer
cataccatgggaaatcgaattttttatctt

SEQ ID NO: 73 - DNA, 76 bp, Artificial Sequence
  primer
catgccatgggaaatcgaattttttatcttttattcgcttttgttcttttgtcggctggtggaagccaaaaaaacg

SEQ ID NO: 74 - DNA, 31 bp, Artificial Sequence
  Primer
ggccatggggaaaaaaatagtatccattagc

SEQ ID NO: 75 - DNA, 80 bp, Artificial Sequence
  Primer
ggccatggggaaaaaaatagtatccattagcttatttttccttatctcagcaactatttggttagccggtaaaaaggaag

SEQ ID NO: 76 - DNA, 35 bp, Artificial Sequence
  Primer
tggttagcctgtgcaaaggaagttgaagaagaacc

SEQ ID NO: 77 - DNA, 35 bp, Artificial Sequence
  primer
ggttcttcttcaacttcctttgcacaggctaacca

SEQ ID NO: 78 - DNA, 37 bp, Artificial Sequence
  primer
ttagcctgtaaagcggaagttgaagaagaaccttttc

SEQ ID NO: 79 - DNA, 37 bp, Artificial Sequence
  primer
gaaaaggttcttcttcaacttccgctttacaggctaa

SEQ ID NO: 80 - DNA, 38 bp, Artificial Sequence
  Primer
gcctgtaaaaaggcagttgaagaagaaccttttctaac

SEQ ID NO: 81 - DNA, 38 bp, Artificial Sequence
  primer
gttagaaaaggttcttcttcaactgcctttttacaggc

SEQ ID NO: 82 - DNA, 35 bp, Artificial Sequence
  primer
tgtaaaaaggaagctgaagaagaaccttttctaac

SEQ ID NO: 83 - DNA, 35 bp, Artificial Sequence
  primer
gttagaaaaggttcttcttcagcttcctttttaca

SEQ ID NO: 84 - DNA, 37 bp, Artificial Sequence
  primer
aaaaaggaagttgcagaagaaccttttctaacaatag

SEQ ID NO: 85 - DNA, 37 bp, Artificial Sequence
  primer
ctattgttagaaaaggttcttctgcaacttccttttt

SEQ ID NO: 86 - DNA, 35 bp, Artificial Sequence
  primer
tggttagcctgtgcagcggaagttgaagaagaacc

SEQ ID NO: 87 - DNA, 35 bp, Artificial Sequence
  primer
ggttcttcttcaacttccgctgcacaggctaacca

SEQ ID NO: 88 - DNA, 38 bp, Artificial Sequence
  primer
gcctgtaaaaaggcagctgcagaagaaccttttctaac

SEQ ID NO: 89 - DNA, 38 bp, Artificial Sequence
  primer
gttagaaaaggttcttctgcagctgcctttttacaggc

SEQ ID NO: 90 - DNA, 37 bp, Artificial Sequence
  primer
aaaaaggaagttgcagcagcaccttttctaacaatag

SEQ ID NO: 91 - DNA, 37 bp, Artificial Sequence
  primer
ctattgttagaaaaggtgctgctgcaacttccttttt

SEQ ID NO: 92 - DNA, 39 bp, Artificial Sequence
  primer
gttgaagaagaagcttttctaacaatagaagaaaaaacc

SEQ ID NO: 93 - DNA, 39 bp, Artificial Sequence
  primer
ggttttttcttctattgttagaaaagcttcttcttcaac

SEQ ID NO: 94 - DNA, 57 bp, Artificial Sequence
  primer
ggctcgagctaagcgtaatctggaacatcgtatgggtaaaacgtaacttgagttctc

SEQ ID NO: 95 - DNA, 35 bp, Artificial Sequence
  primer
ggctcgagttagttcttgataaattcctcaactgg

SEQ ID NO: 96 - DNA, 40 bp, Artificial Sequence
  primer
tggttagcctgtagagcggaagttgaagaagaaccttttc

SEQ ID NO: 97 - DNA, 40 bp, Artificial Sequence
  primer
gaaaaggttcttcttcaacttccgctctacaggctaacca

SEQ ID NO: 98 - DNA, 37 bp, Artificial Sequence
  primer
ttagcctgtgcaagagaagttgaagaagaaccttttc

SEQ ID NO: 99 - DNA, 37 bp, Artificial Sequence
  primer
gaaaaggttcttcttcaacttctcttgcacaggctaa

SEQ ID NO: 100 - DNA, 40 bp, Artificial Sequence
  primer
tggttagcctgtagaagagaagttgaagaagaaccttttc

SEQ ID NO: 101 - DNA, 40 bp, Artificial Sequence
  primer
gaaaaggttcttcttcaacttctcttctacaggctaacca

SEQ ID NO: 102 - DNA, 42 bp, Artificial Sequence
  primer
gcagctgcagcggctccttttctaacaatagaagaaaaaacc

SEQ ID NO: 103 - DNA, 42 bp, Artificial Sequence
  primer
agccgctgcagctgcctttttacaggctaaccaaatagttgc

SEQ ID NO: 104 - DNA, 52 bp, Artificial Sequence
  primer
aaaaaggaagttgaagaagaagtaatcggcggaggcgaatttacacaacccg

SEQ ID NO: 105 - DNA, 46 bp, Artificial Sequence
  primer
ttcttcttcaacttcctttttacaagccgacaaaagaacaaaagcg

SEQ ID NO: 106 - DNA, 46 bp, Artificial Sequence
  primer
aaaaaggaagttgaagtaatcggcggaggcgaatttacacaacccg

SEQ ID NO: 107 - DNA, 40 bp, Artificial Sequence
  primer
ttcaacttcctttttacaagccgacaaaagaacaaaagcg

SEQ ID NO: 108 - DNA, 46 bp, Artificial Sequence
  primer
gcaaaggaagttgaagtaatcggcggaggcgaatttacacaacccg

SEQ ID NO: 109 - DNA, 40 bp, Artificial Sequence
  primer
ttcaacttcctttgcacaagccgacaaaagaacaaaagcg

SEQ ID NO: 110 - DNA, 46 bp, Artificial Sequence
  primer
aaagcggaagttgaagtaatcggcggaggcgaatttacacaacccg

SEQ ID NO: 111 - DNA, 40 bp, Artificial Sequence
  primer
ttcaacttccgctttacaagccgacaaaagaacaaaagcg

SEQ ID NO: 112 - DNA, 44 bp, Artificial Sequence
  primer
aggaagttgaagcagaaccttttctaacaatagaagaaaaaacc

SEQ ID NO: 113 - DNA, 39 bp, Artificial Sequence
  primer
gaaaaggttctgcttcaacttcctttttacaggctaacc

SEQ ID NO: 114 - DNA, 43 bp, Artificial Sequence
  primer
ggaagttgaagaagcaccttttctaacaatagaagaaaaaacc

SEQ ID NO: 115 - DNA, 48 bp, Artificial Sequence
  primer
gaaaaggtgcttcttcaacttcctttttacaggctaaccaaatagttg

SEQ ID NO: 116 - DNA, 46 bp, Artificial Sequence
  primer
aaggaagttgaagaagtaatcggcggaggcgaatttacacaacccg

SEQ ID NO: 117 - DNA, 40 bp, Artificial Seqence
  primer
ttcttcaacttccttacaagccgacaaaagaacaaaagcg

SEQ ID NO: 118 - DNA, 49 bp, Artificial Sequence
  primer
aaaaaggaagttgaagaagtaatcggcggaggcgaatttacacaacccg

SEQ ID NO: 119 - DNA, 43 bp, Artificial Sequence
  primer
ttcttcaacttcctttttacaagccgacaaaagaacaaaagcg

SEQ ID NO: 120 - DNA, 46 bp, Artificial Sequence
  primer
caaaaggacgatgaagtaatcggcggaggcgaatttacacaacccg

SEQ ID NO: 121 - DNA, 40 bp, Artificial Sequence
  primer
ttcatcgtccttttgacaagccgacaaaagaacaaaagcg

SEQ ID NO: 122 - DNA, 46 bp, Artificial Sequence
  primer
gcaaaggacgatgcagtaatcggcggaggcgaatttacacaacccg

SEQ ID NO: 123 - DNA, 40 bp, Artificial Sequence
  primer
tgcatcgtcctttgcacaagccgacaaaagaacaaaagcg

SEQ ID NO: 124 - DNA, 46 bp, Artificial Sequence
  primer
gcaaaggacgatgaagtaatcggcggaggcgaatttacacaacccg

SEQ ID NO: 125 - DNA, 40 bp, Artificial Sequence
  primer
ttcatcgtcctttgcacaagccgacaaaagaacaaaagcg

SEQ ID NO: 126 - DNA, 46 bp, Artificial Sequence
  primer
gcaaaggacgctgcagtaatcggcggaggcgaatttacacaacccg

SEQ ID NO: 127 - DNA, 40 bp, Artificial Sequence
  primer
tgcagcgtcctttgcacaagccgacaaaagaacaaaagcg

SEQ ID NO: 128 - DNA, 46 bp, Artificial Sequence
  primer
gcaaaggaagctgcagtaatcggcggaggcgaatttacacaacccg

SEQ ID NO: 129 - DNA, 40 bp, Artificial Sequence
  primer
tgcagcttcctttgcacaagccgacaaaagaacaaaagcg

SEQ ID NO: 130 - DNA, 46 bp, Artificial Sequence
  primer
gcaaaggaagaggcagtaatcggcggaggcgaatttacacaacccg

SEQ ID NO: 131 - DNA, 40 bp, Artificial Sequence
  primer
tgcctcttcctttgcacaagccgacaaaagaacaaaagcg

SEQ ID NO: 132 - DNA, 46 bp, Artificial Sequence
  primer
gctgcaaaggacgatgtaatcggcggaggcgaatttacacaacccg

SEQ ID NO: 133 - DNA, 40 bp, Artificial Sequence
  primer
atcgtcctttgcagcacaagccgacaaaagaacaaaagcg

SEQ ID NO: 134 - DNA, 49 bp, Artificial Sequence
  primer
gcagctgcaaaggacgatgtaatcggcggaggcgaatttacacaacccg

SEQ ID NO: 135 - DNA, 43 bp, Artificial Sequence
  primer
atcgtcctttgcagctgcacaagccgacaaaagaacaaaagcg

SEQ ID NO: 136 - DNA, 52 bp, Artificial Sequence
  primer
gccgcagctgcaaaggacgatgtaatcggcggaggcgaatttacacaacccg

SEQ ID NO: 137 - DNA, 46 bp, Artificial Sequence
  primer
atcgtcctttgcagctgcggcacaagccgacaaaagaacaaaagcg

SEQ ID NO: 138 - DNA, 46 bp, Artificial Sequence
  primer
tctgatgacttcgaagtaatcggcggaggcgaatttacacaacccg

SEQ ID NO: 139 - DNA, 40 bp, Artificial Sequence
  primer
ttcgaagtcatcagaacaagccgacaaaagaacaaaagcg

SEQ ID NO: 140 - DNA, 46 bp, Artificial Sequence
  primer
caagcggacgatgaagtaatcggcggaggcgaatttacacaacccg

SEQ ID NO: 141 - DNA, 40 bp, Artificial Sequence
  primer
ttcatcgtccgcttgacaagccgacaaaagaacaaaagcg

SEQ ID NO: 142 - DNA, 46 bp, Artificial Sequence
  primer
gcagctgacgatgcagtaatcggcggaggcgaatttacacaacccg

SEQ ID NO: 143 - DNA, 40 bp, Artificial Sequence
  primer
tgcatcgtcagctgcacaagccgacaaaagaacaaaagcg

SEQ ID NO: 144 - DNA, 46 bp, Artificial Sequence
  primer
aaggacgatgcagctgtaatcggcggaggcgaatttacacaacccg

SEQ ID NO: 145 - DNA, 40 bp, Artificial Sequence
  primer
agctgcatcgtccttacaagccgacaaaagaacaaaagcg

SEQ ID NO: 146 - DNA, 46 bp, Artificial Sequence
  primer
agtgatgacgacgatgtaatcggcggaggcgaatttacacaacccg

SEQ ID NO: 147 - DNA, 40 bp, Artificial Sequence
  primer
atcgtcgtcatcactacaagccgacaaaagaacaaaagcg

SEQ ID NO: 148 - protein, 15 aa, C. canimorsus - SiaC
CGSQKNVIGGGEFTI

SEQ ID NO: 149 - protein, 15 aa, Artificial Sequence
  mutated sequence of SEQ ID NO 148
GGSQKNVIGGGEFTI

SEQ ID NO: 150 - protein, 15 aa, Artificial Sequence
  mutated sequence of SEQ ID NO 148
CQKDDEVIGGGEFTI

SEQ ID NO: 151 - protein, 15 aa, Artificial Sequence
  mutated sequence of SEQ ID NO 148
CAKDDEVIGGGEFTI

SEQ ID NO: 152 - protein, 15 aa, Artificial Sequence
  mutated sequence of SEQ ID NO 148
CAKDDAVIGGGEFTI

SEQ ID NO: 153 - protein, 15 aa, Artificial Sequence
  mutated sequence of SEQ ID NO 148
CAKEEAVIGGGEFTI

SEQ ID NO: 154 - protein, 15 aa, Artificial Sequence
  mutated sequence of SEQ ID NO 148
CAKDAAVIGGGEFTI

SEQ ID NO: 155 - protein, 15 aa, Artificial Sequence
  mutated sequence of SEQ ID NO 148
CAKEAAVIGGGEFTI

SEQ ID NO: 156 - protein, 15 aa, Artificial Sequence
  mutated sequence of SEQ ID NO 148
CQADDEVIGGGEFTI

SEQ ID NO: 157 - protein, 15 aa, Artificial Sequence
  mutated sequence of SEQ ID NO 148
CAADDAVIGGGEFTI

SEQ ID NO: 158 - protein, 15 aa, Artificial Sequence
  mutated sequence of SEQ ID NO 148
CGSQKNVIGGGEFTI

SEQ ID NO: 159 - protein, 15 aa, Artificial Sequence
  mutated sequence of SEQ ID NO 148
CAKDDAVIGGGEFTI

SEQ ID NO: 160 - protein, 15 aa, Artificial Sequence
  mutated sequence of SEQ ID NO 148
CAAKDDVIGGGEFTI

SEQ ID NO: 161 - protein, 15 aa, Artificial Sequence
  mutated sequence of SEQ ID NO 148
CAAKDDVIGGGEFTI

SEQ ID NO: 162 - protein, 17 aa, Artificial Sequence
  mutated sequence of SEQ ID NO 148
CAAAAKDDVIGGGEFTI

SEQ ID NO: 163 - protein, 15 aa, Artificial Sequence
  mutated sequence of SEQ ID NO 148
CKDDAAVIGGGEFTI

SEQ ID NO: 164 - protein, 15 aa, Artificial Sequence
  mutated sequence of SEQ ID NO 148
GGSQKNVIGGGEFTI

SEQ ID NO: 165 - protein, 15 aa, Artificial Sequence
  mutated sequence of SEQ ID NO 148
CQKDDEVIGGGEFTI

SEQ ID NO: 166 - protein, 15 aa, Artificial Sequence
  mutated sequence of SEQ ID NO 148
CKKEVEVIGGGEFTI

SEQ ID NO: 167 - protein, 16 aa, Artificial Sequence
  mutated sequence of SEQ ID NO 148
CKKEVEEVIGGGEFTI

SEQ ID NO: 168 - protein, 17 aa, Artificial Sequence
  mutated sequence of SEQ ID NO 148
CKKEVEEEVIGGGEFTI

SEQ ID NO: 169 - protein, 15 aa, Artificial Sequence
  mutated sequence of SEQ ID NO 148
CSDDFEVIGGGEFTI

SEQ ID NO: 170 - protein, 15 aa, Artificial Sequence
  mutated sequence of SEQ ID NO 148
CSDDDDVIGGGEFTI

SEQ ID NO: 171 - protein, 15 aa, Artificial Sequence
  mutated sequence of SEQ ID NO 148
CAKEVEVIGGGEFTI

SEQ ID NO: 172 - protein, 15 aa, Artificial Sequence
  mutated sequence of SEQ ID NO 148
CKAEVEVIGGGEFTI

SEQ ID NO: 173 - protein, 15 aa, Artificial Sequence
  mutated sequence of SEQ ID NO 148
CKEVEEVIGGGEFTI

SEQ ID NO: 174 - protein, 18 aa, C. canimorsus MucG
CKKEVEEEPFLTIEEKTI

SEQ ID NO: 175 - protein, 18 aa, Artificial Sequence
  mutated sequence of SEQ ID NO 174
GKKEVEEEPFLTIEEKTI

SEQ ID NO: 176 - protein, 18 aa, Artificial Sequence
  mutated sequence of SEQ ID NO 174
CAKEVEEEPFLTIEEKTI

SEQ ID NO: 177 - protein, 18 aa, Artificial Sequence
  mutated sequence of SEQ ID NO 174
CKAEVEEEPFLTIEEKTI

SEQ ID NO: 178 - protein, 18 aa, Artificial Sequence
  mutated sequence of SEQ ID NO 174
CKKAVEEEPFLTIEEKTI

SEQ ID NO: 179 - protein, 18 aa, Artificial Sequence
  mutated sequence of SEQ ID NO 174
CKKEAEEEPFLTIEEKTI

SEQ ID NO: 180 - protein, 18 aa, Artificial Sequence
  mutated sequence of SEQ ID NO 174
CKKEVAEEPFLTIEEKTI

SEQ ID NO: 181 - protein, 18 aa, Artificial Sequence
  mutated sequence of SEQ ID NO 174
CKKEVEAEPFLTIEEKTI

SEQ ID NO: 182 - protein, 18 aa, Artificial sequence
  mutated sequence of SEQ ID NO 174
CKKEVEEAPFLTIEEKTI

SEQ ID NO: 183 - protein, 18 aa, Artificial Sequence
  mutated sequence of SEQ ID NO 174
CKKEVEEEAFLTIEEKTI

SEQ ID NO: 184 - protein, 18 aa, Artificial Sequence
  mutated sequence of SEQ ID NO 174
CAAEVEEEPFLTIEEKTI

SEQ ID NO: 185 - protein, 18 aa, Artificial Sequence
  mutated sequence of SEQ ID NO 174
CKKAAAAAPFLTIEEKTI

SEQ ID NO: 186 - protein, 18 aa, Artificial Sequence
  mutated sequence of SEQ ID NO 174
CKKAAAEEPFLTIEEKTI

SEQ ID NO: 187 - protein, 18 aa, Artificial Sequence
  mutated sequence of SEQ ID NO 174
CKKEVAAAPFLTIEEKTI

SEQ ID NO: 188 - protein, 18 aa, Artificial Sequence
  mutated sequence of SEQ ID NO 174
CRREVEEEPFLTIEEKTI

SEQ ID NO: 189 - protein, 18 aa, Artificial Sequence
  mutated sequence of SEQ ID NO 174
CRAEVEEEPFLTIEEKTI

SEQ ID NO: 190 - protein, 18 aa, Artificial Sequence
  mutated sequence of SEQ ID NO 174
CAREVEEEPFLTIEEKTI

SEQ ID NO: 191 - protein, 5 aa, Artificial Sequence
  LES consensus sequence
  MISC_FEATURE (1)..(1): X can be any amino acid
  MISC_FEATURE (5)..(5): X can be any amino acid
XKDEX

SEQ ID NO: 192 - protein, 5 aa, Artificial Sequence
  LES consensus sequence
  MISC_FEATURE (1)..(1): X can be any amino acid
  MISC_FEATURE (5)..(5): X can be any amino acid
XKEDX

SEQ ID NO: 193 - protein, 5 aa, Artificial Sequence
  LES consensus sequence
  MISC_FEATURE (1)..(1): X can be any amino acid
  MISC_FEATURE (5)..(5): X can be any amino acid
XKDDX

SEQ ID NO: 194 - protein, 5 aa, Artificial Sequence
  LES consensus sequence
  MISC_FEATURE (1)..(1): X can be any amino acid
  MISC_FEATURE (5)..(5): X can be any amino acid
XKEEX

SEQ ID NO: 195 - protein, 19 aa, C. canimorsus 5
MNRIFYLLFAFVLLSACGS

SEQ ID NO: 196 - protein, 22 aa, C. canimorsus 5
MKKIVSISLFFLISATIWLACK

SEQ ID NO: 197 - protein, 4 aa, Artificial Sequence
  LES consensus sequence
  MISC_FEATURE (1)..(1): X can be any amino acid
  MISC_FEATURE (2)..(2): X is selected from K and A, with the proviso that when X(2) is A, X(1) is Q
  MISC_FEATURE (3)..(4): X is selected from D and E
XXXX

SEQ ID NO: 198 - protein, 5 aa, Artificial Sequence
  LES consensus sequence
  MISC_FEATURE (1)..(1): X is selected from S and T
  MISC_FEATURE (2)..(3): X is selected from D and E
  MISC_FEATURE (4)..(4): X is selected from D, E and F
  MISC_FEATURE (5)..(5): X is selected from D and E
XXXXX

SEQ ID NO: 199 - protein, 5 aa, Artificial Sequence
  LES consensus sequence
  MISC_FEATURE (1)..(1): X can be any amino acid
  MISC_FEATURE (4)..(4): X can be any amino acid
XKEXE

SEQ ID NO: 200 - protein, 6 aa, Artificial Sequence
  LES consensus sequence
  MISC_FEATURE (1)..(1): X can be any amino acid
  MISC_FEATURE (4)..(4): X can be any amino acid
XKEXEE

SEQ ID NO: 201 - protein, 5 aa, Artificial Sequence
  LES consensus sequence
  MISC_FEATURE (1)..(1): X can be any amino acid
XKEVE

SEQ ID NO: 202 - protein, 6 aa, Artificial Sequence
  LES consensus sequence
  MISC_FEATURE (1)..(1): X can be any amino acid
XKEVEE

SEQ ID NO: 203 - protein, 4 aa, Artificial Sequence
  lipobox motif
  MISC_FEATURE (2)..(2): X is selected from S and A
  MISC_FEATURE (3)..(3): X is selected from A and G
LXXC

SEQ ID NO: 204 - protein, 7 aa, Artificial Sequence
  protease cleavage site
ENLYFQG

SEQ ID NO: 205 - protein, 5 aa, Artificial Sequence
  protease cleavage site
DDDDK

SEQ ID NO: 206 - protein, 6 aa, Artificial Sequence
  protease cleavage site
LVPRGS

(19) United States
(12) Patent Application Publication
(10) Pub. No.: US 2019/0194264 A1
(43) Pub. Date: Jun. 27, 2019

(54) LIPOPROTEIN EXPORT SIGNALS AND USES THEREOF

(71) Applicant: UNIVERSITÉ DE NAMUR, Namur (BE)

(72) Inventors: Frédéric André Lauber, Biederthal (FR); Francesco Renzi, Namur (BE); Guy Richard Comelis, Crupet (BE)

(73) Assignee: UNIVERSITÉ DE NAMUR, Namur (BE)

(21) Appl. No.: 16/319,835

(22) PCT Filed: Aug. 11, 2017
(86) PCT No.: PCT/EP2017/070408

§ 371 Date: Jan. 23, 2019

(30) Foreign Application Priority Data
Aug. 12, 2016 (EP) 16183962.6

(51) Int. Cl. C07K 14/195; C12P 21/02

(52) U.S. Cl. CPC C07K 14/195; C12P 21/02; C12Y 402/02001; C12Y 302/01018; C07K 2319/035

(57) ABSTRACT

Described herein are polypeptides comprising a lipoprotein export signal, polypeptide precursors comprising a lipoprotein export signal, nucleic acids encoding said polypeptides or polypeptide precursors, recombinant expression vectors comprising nucleic acids encoding said lipoprotein export signal and/or polypeptides or polypeptide precursors, and 5 recombinant host cells comprising these vectors. The application further provides uses of these polypeptides, polypeptide precursors, nucleic acids, recombinant expression vectors and recombinant host cells.

### TECHNICAL FIELD

The present invention is situated in the field of lipoprotein signal peptides. More particularly, the invention provides polypeptides comprising these signal peptides, uses thereof, nucleic acids encoding said polypeptides, nucleic acid constructs comprising the nucleic acid sequence encoding these peptides and recombinant expression vectors and recombinant host cells comprising these nucleic acid constructs.

### BACKGROUND OF THE INVENTION

Cell surface display allows expression of proteins or peptides, or fragments thereof, on the surface of cells in a stable manner using the surface proteins of bacteria, yeast, or even mammalian cells as anchoring motifs. This powerful tool has been used in a wide range of biotechnological and industrial applications, such as live or inactivated vaccine development to expose heterologous epitopes on human commensal or attenuated pathogenic bacterial cells to elicit antigen-specific antibody responses, screening-displayed peptide libraries, antibody production by expressing surface antigens to raise polyclonal antibodies in animals, whole-cell catalysis by immobilizing enzymes, biosensor development and environmental bio adsorption for removal of harmful chemicals and heavy metals.

In the mid-eighties, George P. Smith was the first to develop a surface expression system, by displaying on the surface of a bacteriophage the peptides and small proteins fused with the pill protein of the filamentous phage. Since then, various phage display systems have been developed to express foreign proteins on the surface of the phage. However, the size of foreign protein to be displayed on the surface of phage is rather limited. As a result hereof, the microbial cell-surface display system was developed. Microbial cell-surface display is carried out by expressing a heterologous peptide or protein of interest as a fusion protein with various anchoring motifs, which are usually cell-surface proteins or their fragments (‘carrier proteins’).

Typically, the use of carrier proteins can influence the cell physiology. For example, the use of outer membrane (OM) proteins and subunits of cellular appendages might lead to growth defects and destabilization of cell envelope integrity. Additionally, a successful carrier should not become unstable on the insertion or fusion of heterologous sequences and it should be resistant to attack by proteases present in the periplasmic space or medium.

Various anchoring motifs have been developed, including OprF, OmpC, OmpX, the outer membrane protein S, maltoprotein LamB and lipoprotein TraT. Although many successful results have been achieved, the use of current anchoring motifs did not always allow efficient display of all target proteins. In cell surface display systems, successful protein display is highly dependent on the choice of the anchoring motif. Thus, there is a high need to explore and develop new and improved cell surface display systems for the expression and display of recombinant proteins.

### SUMMARY OF THE INVENTION

The inventors have found a new consensus sequence motif specific for surface-exposed lipoproteins, said specific motif acting as a lipoprotein export signal (LES). Polypeptides comprising such a LES can be successfully exported and displayed to the cell surface of a host cell with high efficiency and stability.

Accordingly, provided herein is a polypeptide precursor comprising

(a) an N-terminal signal peptide of a lipoprotein of Gram-negative bacteria comprising a lipobox motif located at the very end of the C-terminus of said signal peptide, wherein said lipobox motif consists of the amino acid sequence L(S/A)(A/G)C (SEQ ID NO: 230) and is specifically recognizable by a signal peptidase type II;

(b) a lipoprotein export signal comprising an amino acid sequence according to any one of the following consensus sequences:
XJZZ (SEQ ID NO: 197), wherein X can be any amino acid, wherein J is selected from the group consisting of K and A, wherein Z is selected from the group consisting of D and E, with the proviso that when J is A, X is Q;
BZZUZ (SEQ ID NO: 198), wherein B is selected from the group consisting of S and T, wherein Z is selected from the group consisting of D and E and wherein U is selected from the group consisting of D, E and F; or
XKEOEE (SEQ ID NO: 200), wherein X and O can be any amino acid, preferably wherein O is V;

wherein said lipoprotein export signal is overall negatively charged and wherein said lipoprotein export signal is located directly adjacent to the C-terminus of said signal peptide;

(c) a polypeptide, wherein said polypeptide is located C-terminally of said signal peptide and said lipoprotein export signal; and

(d) optionally, a protease cleavage site motif, wherein said protease cleavage site motif is different from said lipobox motif and is located C-terminally of said signal peptide and said lipoprotein export signal and N-terminally of said polypeptide;

wherein said signal peptide, said lipoprotein export signal and said polypeptide do not naturally occur together in a polypeptide sequence. In particular embodiments, said N-terminal signal peptide of a lipoprotein of Gram-negative bacteria is the signal peptide of sialidase (siaC) or mucinase (MucG) of C. canimorsus 5. In particular embodiments, lipoprotein export signal is selected from an amino acid sequence according to any one of SEQ ID NO: 16 to SEQ ID NO: 20 or SEQ ID NO: 40 to 47; any one of SEQ ID NO:1 to SEQ ID NO: 15 or SEQ ID NO: 25 to 39; or any one of SEQ ID NO:49 to SEQ ID NO:51 or SEQ ID NO:63.

Also provided herein is a nucleic acid encoding the polypeptide precursor as described herein.

Also provided herein is a recombinant expression vector comprising the nucleic acid as described herein, a promoter and transcriptional and translational stop signals, and optionally a selectable marker.

Also provided herein is a recombinant expression vector comprising

(a) a nucleic acid sequence encoding a signal peptide of a lipoprotein of Gram-negative bacteria wherein said signal peptide comprises a lipobox motif located at the very end of the C-terminus of said signal peptide, wherein said lipobox motif consists of the amino acid sequence L(S/A)(A/G)C and is specifically recognized by a signal peptidase type II;

(b) a nucleic acid sequence encoding a lipoprotein export signal having an amino acid sequence according to any one of the following consensus sequences:
XJZZ, wherein X can be any amino acid, wherein J is selected from the group consisting of K and A, wherein Z is selected from the group consisting of D and E; with the proviso that when J is A, X is Q;
BZZUZ, wherein B is selected from the group consisting of S and T, wherein Z is selected from the group consisting of D and E and wherein U is selected from the group consisting of D, E and F; or
XKEOEE, wherein X and O can be any amino acid, preferably wherein O is V;

wherein said lipoprotein export signal is overall negatively charged and wherein said nucleic acid sequence encoding said lipoprotein export signal is located directly downstream of said nucleic acid sequence encoding said signal peptide;

(c) optionally, a nucleic acid sequence encoding a protease cleavage site motif, wherein said protease cleavage site motif is different from said lipobox motif and is located downstream of said nucleic acid sequence encoding said lipoprotein export signal and said nucleic acid sequence encoding said signal peptide; and

(d) a multiple cloning site, wherein said multiple cloning site is located downstream of said nucleic acid encoding said lipoprotein export signal and said nucleic acid encoding said signal peptide and, optionally downstream of said protease cleavage site motif. In particular embodiments, said N-terminal signal peptide of a lipoprotein of Gram-negative bacteria is the signal peptide of sialidase (siaC) or mucinase (MucG) of C. canimorsus 5. In particular embodiments, said lipoprotein export signal is selected from an amino acid sequence according to any one of SEQ ID NO: 16 to SEQ ID NO: 20 or SEQ ID NO: 40 to 47; any one of SEQ ID NO: 1 to SEQ ID NO: 15 or SEQ ID NO: 25 to 39; or any one of SEQ ID NO: 49 to SEQ ID NO: 51 or SEQ ID NO: 63.

Also provided herein is a recombinant host cell comprising the vector as described herein, wherein said host cell is a bacterial cell of the Bacteroidetes phylum. In particular embodiments, said bacterial cell of the Bacteroidetes phylum is Capnocytophaga canimorsus or Flavobacterium johnsoniae.

Another aspect relates to the use of a lipoprotein export signal comprising an amino acid sequence according to one of the following consensus sequences:
XJZZ, wherein X can be any amino acid, wherein J is selected from the group consisting of K and A, wherein Z is selected from the group consisting of D and E; with the proviso that when J is A, X is Q;
BZZUZ, wherein B is selected from the group consisting of S and T, wherein Z is selected from the group consisting of D and E and wherein U is selected from the group consisting of D, E and F; or
XKEOEE, wherein X and O can be any amino acid, preferably wherein O is V;

wherein said lipoprotein export signal is overall negatively charged and wherein said lipoprotein export signal is located directly adjacent to an N-terminal lipid-modified cysteine residue originating from an N-terminal signal peptide of a lipoprotein of Gram-negative bacteria comprising a lipobox motif located at the very end of the C-terminus of said signal peptide, wherein said lipobox motif consists of the amino acid sequence L(S/A)(A/G)C and is specifically recognizable by a signal peptidase type II, for surface exposure of a polypeptide in a host cell, wherein said polypeptide originates from the same or a different organism than said host cell and wherein said lipoprotein export signal and said polypeptide do not naturally occur together in a polypeptide sequence. In particular embodiments, said N-terminal signal peptide of a lipoprotein of Gram-negative bacteria is the signal peptide of sialidase (siaC) or mucinase (MucG) of C. canimorsus 5. In particular embodiments, said lipoprotein export signal is selected from an amino acid sequence according to any one of SEQ ID NO: 16 to SEQ ID NO: 20 or SEQ ID NO: 40 to 47; any one of SEQ ID NO: 1 to SEQ ID NO: 15 or SEQ ID NO: 25 to 39; or any one of SEQ ID NO: 49 to SEQ ID NO: 51 or SEQ ID NO: 63.

Also provided herein is the use of

(i) the polypeptide precursor as described herein above,

(ii) the nucleic acid as described herein above,

(iii) the expression vector as described herein above; or

(iv) the host cell as described herein above,

for manufacturing a vaccine, for producing antibodies, for biosorption applications, for manufacturing biosensors, for performing bacterial display, for whole-cell based biocatalytic applications or for protein production and purification, wherein said production of antibodies is not a method of treatment. In particular embodiments, said polypeptide precursor comprises and/or said nucleic acid or said expression vector encodes an antigen, or epitope thereof, or an enzyme, or catalytically active fragment thereof, which will be exposed to the surface of a bacterial cell of the Bacteroidetes phylum comprising said polypeptide precursor, said nucleic acid and/or said expression vector. In particular embodiments, said bacterial cell of the Bacteroidetes phylum is Capnocytophaga canimorsus or Flavobacterium johnsoniae.

### BRIEF DESCRIPTION OF THE FIGURES

FIG. 1. Multiple sequence alignment of C. canimorsus lipoproteins. (A) MAFFT alignment of mature surface exposed lipoproteins. Only the N-terminal region, showing the conserved K-(D/E) motif, is displayed. Highly conserved residues are highlighted. The derived consensus sequence is shown below. (B). MAFFT alignment of the first 15 N-terminal amino acids of intracellular outer membrane (OM) mature lipoproteins. The first invariant cysteine residue of each sequence was removed before performing the alignment. Highly conserved residues are highlighted. The derived consensus sequence is shown below. Sialidase (SiaC; Ccan_04790) is indicated by a star.

FIG. 2. Alignment of C. canimorsus surface exposed lipoproteins reveals the presence of an N-terminal conserved motif. (A) Sequence alignment of the first 15 N-terminal amino acids of mature surface exposed lipoproteins. The first invariant cysteine residue of each sequence was removed before performing the alignment. Highly conserved residues are highlighted. The derived consensus sequence is shown below. Mucinase (MucG) is indicated by a star. (B) Generated WebLogo of the consensus sequence determined in A. Positions relative to the +1 cysteine are indicated below. (C) Amino acid frequency for each position of the consensus sequence, expressed in percentage. The three most represented amino acids for each position are shown.

FIG. 3. The LES allows SiaC surface exposure. (A) Sialidase (SiaC) wt and consensus sequence mutant constructs. Amino acids derived from the consensus are indicated in dark gray, point mutations are indicated in light grey. (B) Western blot analysis of total cell extracts of strains expressing the SiaC constructs described in A. Expression of Mucinase (MucG) was monitored as loading control. (C) Quantification of SiaC surface exposure by flow cytometry of live cells labeled with anti-SiaC serum. The percentage of labeled cells is indicated below. Strains below detection limit (NR, not relevant; <2.5%) are highlighted in grey, strains with a statistically lower stained population are in grey. Shown is the mean fluorescent intensity (MFI). The averages from three independent experiments are shown. Error bars represent 1 standard deviation from the mean. (D) Immunofluorescence microscopy images of bacteria labeled with anti-SiaC serum. Scale bar: 5 μm. (E) Detection of SiaC by western blot analysis of total lysates (TL) and outer membrane (OM) fractions of bacteria expressing different SiaC constructs. Expression of MucG was monitored as loading control.

FIG. 4. The position of the minimal LES is crucial for its function.

(A) Sialidase (SiaC) wt and consensus sequence mutant constructs. Amino acids derived from the consensus are indicated in dark grey, point mutations are indicated in light grey. (B) Detection of SiaC by western blot analysis of total cell extracts of strains expressing the SiaC constructs described in (A) Mucinase (MucG) expression was monitored as loading control. (C) Quantification of SiaC surface exposure by flow cytometry of live cells labeled with anti-SiaC serum. Shown is the fluorescence intensity of stained cells only; NR: not relevant. The averages from at least three independent experiments are shown. Error bars represent 1 standard deviation from the mean; ***, p≤0.001. The percentage of stained cells is indicated below; SD: standard deviation. Strains below detection limit (≤2.5%) are highlighted in grey, strains with a statistically significant lower stained population are in grey. (D) Immunofluorescence microscopy images of bacteria stained with anti-SiaC serum. Scale bar: 5 μm. (E) Western blot analysis of total lysate (TL) and outer membrane (OM) fraction of bacteria expressing different SiaC constructs. MucG expression was monitored as loading control.

FIG. 5. MucG is a surface exposed lipoprotein (A) Mucinase (MucG) domain annotation. Predicted structural domains are indicated by grey boxes, amino acid positions are indicated on top. The predicted lipoprotein export signal (LES) is shown below. (B) Western blot analysis (top) and fluorography (bottom) of the elution fraction of MucG immunoprecipitation of ³H palmitate labeled bacteria. MucG is lipidated in the wt and ΔmucG+MucG strains but not in the ΔmucG+MucG_{C21G} strain in which the predicted site of lipidation is mutated, showing that MucG is a lipoprotein. (C) MucG detection by western blot analysis of total cell lysates (TL) and outer membrane (OM) fractions of bacteria expressing different MucG constructs. MucG but not the soluble MucG_{C21G} is detected in the OM fraction, showing that MucG is a bona fide OM lipoprotein. SiaC expression was monitored as loading control. (D) Quantification of MucG surface exposure by flow cytometry of live cells labeled with anti-MucG serum. Shown is the fluorescence intensity of stained cells only; NR: not relevant. The averages from at least three independent experiments are shown. Error bars represent 1 standard deviation from the mean; ***, p≤0.001. The percentage of stained cells is indicated below; SD: standard deviation. Strains below detection limit (≤2.5%) are highlighted in grey. (E) Immunofluorescence microscopy pictures of bacteria labeled with anti-MucG serum. Scale bar: 5 μm. (F) Detection of mucin by PNA lectin staining of human saliva following incubation with bacteria expressing different MucG constructs. Untreated saliva serves as negative control. Reduction of PNA staining indicates mucin degradation by surface localized MucG.

FIG. 6. Addition of the MucG LES leads to surface exposure of SiaC. (A) Sialidase (SiaC) wt and mucinase (MucG) consensus sequence mutant constructs. Amino acids derived from the MucG consensus are indicated in dark grey, point mutations are indicated in grey. (B) Detection of SiaC by western blot analysis of total cell extracts of strains expressing the SiaC constructs shown in (A). MucG expression was monitored as loading control. (C) Quantification of SiaC surface exposure by flow cytometry of live cells labeled with anti-SiaC serum. Shown is the fluorescence intensity of stained cells only; NR: not relevant. The averages from at least three independent experiments are shown. Error bars represent 1 standard deviation from the mean; ***, p≤0.001. The percentage of stained cells is indicated below; SD: standard deviation. Strains below detection limit (≤2.5%) are shaded in grey, strains with a statistically significant lower stained population are in grey. (D) Immunofluorescence microscopy pictures of bacteria labeled with anti-SiaC serum. Scale bar: 5 μm. (E) Western blot analysis of total lysate (TL) and outer membrane (OM) fraction of bacteria expressing different SiaC constructs. MucG expression was monitored as loading control.

FIG. 7. Multiple sequence alignment of B. fragilis and F. johnsoniae lipoproteins. (A) (A-C) MAFFT alignment of the first 16 N-terminal amino acids of proteinase K sensitive B. fragilis lipoproteins. Highly conserved residues are highlighted. Corresponding Weblogo and amino acid frequencies are indicated below. (D-F) MAFFT alignment of the first 16 N-terminal amino acids of SusD-like F. johnsoniae lipoproteins. Highly conserved residues are highlighted. Corresponding Weblogo and amino acid frequencies are indicated below.

FIG. 8. B. fragilis and F. johnsoniae LES allow SiaC surface localization. (A) Sialidase (SiaC) wt and consensus sequence mutant constructs. Amino acids derived from the B. fragilis or F. johnsoniae consensus are indicated in dark grey, point mutations are indicated in light grey. (B) Detection of SiaC by western blot analysis of total cell extracts of strains expressing the SiaC constructs described in (A). Mucinase (MucG) expression was monitored as loading control. (C) Quantification of SiaC surface exposure by flow cytometry of live cells labeled with anti-SiaC serum. Shown is the fluorescence intensity of stained cells only; NR: not relevant. The averages from at least three independent experiments are shown. Error bars represent 1 standard deviation from the mean; ***, p≤0.001. The percentage of stained cells is indicated below; SD: standard deviation. Strains below detection limit (≤2.5%) are highlighted in grey. (D) Immunofluorescence microscopy images of bacteria labeled with anti-SiaC serum. Scale bar: 5 μm.

FIG. 9. Characterization of the MucG LES in SiaC (A) Sialidase (SiaC) wt and Mucinase (MucG) LES sequence mutant constructs. Amino acids derived from MucG are indicated in dark grey, point mutations are indicated in light grey. (B) Detection of SiaC by western blot analysis of total cell extracts of strains expressing the SiaC constructs described in (A). Expression of MucG was monitored as loading control. (C) Quantification of SiaC surface exposure by flow cytometry of live cells labeled with anti-SiaC serum. Shown is the fluorescence intensity of stained cells only; NR: not relevant. The averages from at least three independent experiments are shown. Error bars represent 1 standard deviation from the mean; ***, p≤0.001. The percentage of stained cells is indicated below; SD: standard deviation. Strains below detection limit (2.5%) are highlighted in grey, strains with a statistically significant lower stained population are in grey.

FIG. 10. MucG LES mutational analysis—single substitutions (A) Mucinase (MucG) wt and mutant constructs. Point mutations are indicated in light grey. (B) Detection of MucG by western blot analysis of total cell extracts of strains expressing the MucG constructs described in (A). Expression of sialidase (SiaC) was monitored as loading control. (C) Quantification of MucG surface exposure by flow cytometry of live cells labeled with anti-MucG serum. Shown is the fluorescence intensity of stained cells only; NR: not relevant. The averages from at least three independent experiments are shown. Error bars represent 1 standard deviation from the mean; ***, p≤0.001. The percentage of stained cells is indicated below; SD: standard deviation. Strains below detection limit (2.5%) are highlighted in grey.

FIG. 11. MucG LES mutational analysis—multiple substitutions (A) Mucinase (MucG) wt and mutant constructs. Point mutations are indicated in light grey. (B) Detection of MucG by western blot analysis of total cell extracts of strains expressing the MucG constructs described in (A). Expression of sialidase (SiaC) was monitored as loading control. (C) Quantification of MucG surface exposure by flow cytometry of live cells labeled with anti-MucG serum. Shown is the fluorescence intensity of stained cells only; NR: not relevant. The averages from at least three independent experiments are shown. Error bars represent 1 standard deviation from the mean; ***, p≤0.001. The percentage of stained cells is indicated below; SD: standard deviation. Strains below detection limit (2.5%) are highlighted in grey, strains with a statistically significant lower stained population are in grey.

FIG. 12. Arginine can functionally replace lysine in the MucG LES (A) Mucinase (MucG) wt and mutant constructs. Arginine substitutions are indicated in dark grey, alanine substitutions are indicated in light grey. (B) Quantification of MucG surface exposure by flow cytometry of live cells labeled with anti-MucG serum. Shown is the fluorescence intensity of stained cells only; NR: not relevant. The averages from at least three independent experiments are shown. Error bars represent 1 standard deviation from the mean; ***, p≤0.001. The percentage of stained cells is indicated below; SD: standard deviation. Strains below detection limit (2.5%) are highlighted in grey, strains with a statistically significant lower stained population are in grey.

FIG. 13. Exemplary schematic overview of surface-exposed lipoprotein biogenesis and transport pathways in a host cell of the Bacteroidetes phylum. The polypeptide precursor comprising an N-terminal signal peptide, a LES and a polypeptide as described herein is inserted into the inner membrane by the Sec translocase. The lipobox motif comprised within the N-terminal signal peptide is recognized by the lipoprotein diacylglyceryl transferase (Lgt) that attaches a diacylglyceryl moiety, to the SH of the +1 cysteine. Then, the signal peptide is cleaved by the type II signal peptidase (SPase II). Following signal peptide cleavage, the N-terminal cysteine residue is modified with an additional acyl chain by the lipoprotein N-acyl-transferase (Lnt). The mature lipoprotein is extracted from the inner membrane and transported across the periplasm to the outer membrane by the Lol system and finally inserted into the outer membrane and translocated to the bacterial surface by an unknown mechanism (indicated by dashed lines).

### DETAILED DESCRIPTION OF THE INVENTION

Before the present uses of these peptides, kits comprising these polypeptides, polypeptide precursors, nucleic acid constructs comprising the nucleic acid sequence encoding these polypeptides and/or polypeptide precursors and recombinant expression vectors and recombinant host cells comprising these nucleic acid constructs used in the invention are described, it is to be understood that this invention is not limited to particular polypeptides, polypeptides precursors, uses, nucleic acid constructs, vectors and host cells described, as such particular polypeptides, polypeptide precursors, uses, nucleic acid constructs, vectors and host cells may, of course, vary. It is also to be understood that the terminology used herein is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein may be used in the practice or testing of the present invention, the preferred methods and materials are now described.

In this specification and the appended claims, the singular forms “a”, “an”, and “the” include plural references unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps.

The terms “comprising”, “comprises” and “comprised of” also include the term “consisting of”.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

The recitation of numerical ranges by endpoints includes all numbers and fractions subsumed within the respective ranges, as well as the recited endpoints.

The term “amino acid” as used herein generally refers to a molecule that contains both amine and carboxyl functional groups. In biochemistry, this term particularly refers to alpha-amino acids with the general formula H₂NCHRCOOH, where R is an organic substituent. In the alpha-amino acids, the amino and carboxylate groups are attached to the same carbon, i.e., the α-carbon. The term includes the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, norvaline, norleucine and ornithine. The term includes both D- and L-amino acids. L-amino acids are preferred. Within this application, amino acids are referred to by their 1-letter code or their full name. For example, cysteine can be referred to as cysteine or C.

The abbreviations G, A, L, M, F, W, K, Q E, S, P, V, I, C, Y, H, R, N, D, T, as used herein correspond to the single-letter amino acid codes as known in the art and reproduced below:

| One letter code | Amino acid | Three letter code |
|---|---|---|
| G | Glycine | Gly |
| A | Alanine | Ala |
| L | Leucine | Leu |
| M | Methionine | Met |
| F | Phenylalanine | Phe |
| W | Tryptophan | Trp |
| K | Lysine | Lys |
| Q | Glutamine | Gln |
| E | Glutamic Acid | Glu |
| S | Serine | Ser |
| P | Proline | Pro |
| V | Valine | Val |
| I | Isoleucine | Ile |
| C | Cysteine | Cys |
| Y | Tyrosine | Tyr |
| H | Histidine | His |
| R | Arginine | Arg |
| N | Asparagine | Asn |
| D | Aspartic Acid | Asp |
| T | Threonine | Thr |

The abbreviations B, J, O, U, X, Y and Z, and X₁-X₁₀ are used to indicate variable amino acids, whereby the nature of the variation is as specified herein.

The terms “peptide”, “polypeptide”, or “protein” can be used interchangeably and relate to any natural, synthetic, or recombinant molecule comprising amino acids joined together by peptide bonds between adjacent amino acid residues. A “peptide bond”, “peptide link” or “amide bond” is a covalent bond formed between two amino acids when the carboxyl group of one amino acid reacts with the amino group of the other amino acid, thereby releasing a molecule of water. The polypeptide can be from any source, e.g., a naturally occurring polypeptide, a chemically synthesized polypeptide, a polypeptide produced by recombinant molecular genetic techniques, or a polypeptide from a cell or translation system. Preferably, the polypeptide is a polypeptide produced by recombinant molecular genetic techniques. The polypeptide may be a linear chain or may be folded into a globular form. The terms “amino acid” and “amino acid residue” may be used interchangeably herein. The term peptide, polypeptide or protein encompasses fragments of full length proteins.

The term “functionally active polypeptide, protein or peptide” as used herein refers to the form of the polypeptide, protein or peptide which can exert an intended function. For example, the functionally active form of an enzyme can accelerate or catalyse chemical reactions. The functionally active polypeptide can be homologous (originating from the same organism) or heterologous (originating from a different organism) to the host cell.

The term “fragment” of a protein refers to N-terminally and/or C-terminally deleted or truncated forms of said protein. The term encompasses fragments arising by any mechanism, such as, without limitation, by alternative translation, exo- and/or endo-proteolysis and/or degradation of said protein, such as, for example, in vivo or in vitro, such as, for example, by physical, chemical and/or enzymatic proteolysis. Without limitation, a fragment of a protein may represent at least about 5% (by amino acid number), or at least about 10%, e.g., 20% or more, 30% or more, or 40% or more, such as preferably 50% or more, e.g., 60% or more, 70% or more, 80% or more, 90% or more, or 95% or more of the amino acid sequence of said protein.

Where the present specification refers to or encompasses fragments of proteins, this includes fragments which are functionally active or functional, i.e., which at least partly retain the biological activity or intended functionality of the respective or corresponding proteins, polypeptides, or peptides. In particular embodiments, the fragments or polypeptides at least partly retain the antigenic properties of the corresponding protein.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

Gram-negative bacteria are a group of bacteria which are characterized by their cell membranes, which are composed of a thin peptidoglycan cell wall sandwiched between an inner cytoplasmic cell membrane and a bacterial outer membrane (OM). Gram-negative bacteria include not only Proteobacteria but also the vast phylum Bacteroidetes. Presently, the Inventors found a signal that targets lipoproteins from several classes of the Bacteroidetes phylum to the cell surface. More particularly, the Inventors have found new consensus sequence motifs specific for surface-exposed lipoproteins, namely
X₁X₂(D/E)₂ (SEQ ID NO: 68-71), wherein X₁ can be any amino acid and X₂ is selected from the group consisting of K, S, T and A, with the proviso that when X₂ is A, X₁ is Q;
XJZZ (SEQ ID NO: 197), wherein X can be any amino acid, wherein J is selected from the group consisting of K and A, wherein Z is selected from the group consisting of D and E; with the proviso that when J is A, X is Q;
BZZUZ (SEQ ID NO: 198), wherein B is selected from the group consisting of S and T, wherein Z is selected from the group consisting of D and E and wherein U is selected from the group consisting of D, E and F; or
XKEOE (SEQ ID NO: 199), preferably XKEOEE (SEQ ID NO: 200), wherein X and O can be any amino acid, preferably wherein O is V; said specific motifs acting as lipoprotein export signals (LES). Additionally, polypeptides comprising said LES were successfully secreted and displayed to the cell surface with high efficiency and stability.

It is noted that the letters X, J, Z, B and O used in the consensus sequences as described herein which do not represent the abbreviation of one of the 20 naturally occurring amino acids but represent variable amino acids can alternatively be referred to herein as “Xₙ”, wherein “n” is a natural number other than 1 or 2. For example, “X” can be referred to as “X₅”, “J” can be referred to as “X₆”, “Z” can be referred to as “X₇”, “B” can be referred to as “X₈”, “U” can be referred to as “X₉” and “O” can be referred to as “X₁₀”. Similarly, where an amino acid is represented as being one of two options, such as E/D, S/A or NG, these options can also be represented by a specific Xₙ.

The application thus relates to polypeptides comprising said LES. Accordingly, a first aspect of the invention relates to a polypeptide comprising:

(a) a lipoprotein export signal located within the first 15 amino acids of the N-terminal region of said polypeptide, wherein said lipoprotein export signal comprises an amino acid sequence according to any one of the following consensus sequences: X₁X₂DD (SEQ ID NO: 68), X₁X₂DE (SEQ ID NO: 69), X₁X₂ED (SEQ ID NO: 70) or X₁X₂EE (SEQ ID NO: 71), wherein X₁ can be any amino acid and X₂ is selected from the group consisting of K, S, T and A, with the proviso that when X₂ is A, X₁ is Q;

(b) a functionally active polypeptide or fragment thereof; and

(c) optionally, a protease cleavage site motif C-terminally of said lipoprotein export signal and N-terminally of said functionally active polypeptide or fragment thereof.

In particular embodiments, said protein is a mature protein originating from a precursor polypeptide, which is a polypeptide comprising an N-terminal signal peptide linked to a protein. Such precursor polypeptides typically comprise, within the N-terminal signal peptide, a lipobox motif which is cleavable by signal peptidase type II. As a result thereof, the mature protein originating from said precursor protein by cleavage of signal peptidase type II will comprise a +1 cysteine, which is a remnant of the lipobox motif. Accordingly, in particular embodiments, the mature polypeptides comprise a +1 cysteine N-terminally of said lipoprotein export signal. It is noted that in this context amino acid position “+1” refers to the first amino acid after (or C-terminally from) the cleavage site of the signal peptidase. In mature lipoproteins originating from precursor proteins as described herein this will correspond to the first amino acid residue of the mature lipoproteins

The invention further also relates to a mature polypeptide comprising:

(a) optionally, an N-terminal cysteine residue, preferably wherein said cysteine residue is lipid-modified;

(b) a lipoprotein export signal comprising the amino acid sequence according to any one of the following consensus sequences:
XJZZ (SEQ ID NO: 197), wherein X can be any amino acid, wherein J is selected from the group consisting of K and A, wherein Z is selected from the group consisting of D and E; with the proviso that when J is A, X is Q;
BZZUZ (SEQ ID NO: 198), wherein B is selected from the group consisting of S and T, wherein Z is selected from the group consisting of D and E and wherein U is selected from the group consisting of D, E and F; or
XKEOE (SEQ ID NO: 199), preferably XKEOEE (SEQ ID NO: 200), wherein X and O can be any amino acid, preferably wherein O is V;

preferably XJZZ, wherein X can be any amino acid, wherein J is selected from the group consisting of K and A, wherein Z is selected from the group consisting of D and E; with the proviso that when J is A, X is Q;

wherein said lipoprotein export signal is located directly C-terminally of said cysteine residue;

(c) a polypeptide, wherein said polypeptide is located C-terminally of said lipoprotein export signal and said cysteine residue; and

(d) optionally, a protease cleavage site motif which is located C-terminally of said lipoprotein export signal and N-terminally of said polypeptide.

As indicated above, in particular embodiments, said N-terminal cysteine residue is the conserved +1 cysteine of the lipobox motif, which originates from cleavage of the N-terminal signal peptide comprising said lipobox motif from the polypeptide precursor by a signal peptidase type II (SPaseII).

In particular embodiments, said lipoprotein export signal is overall negatively charged.

In particular embodiments, said N-terminal cysteine residue, said lipoprotein export signal and said polypeptide do not naturally occur together in a polypeptide sequence.

In particular embodiments, the polypeptide, such as the functionally active polypeptide or fragment thereof, is linked to an N-terminal or C-terminal tag.

The “lipoprotein export signal” or “LES” as herein thus refers to a short amino acid sequence of at least 3 amino acid residues, and preferably at most 30 amino acid residues, that is derived from a lipoprotein and acts as a signal peptide that targets the lipoprotein for export to the cell surface of a Gram-negative bacterial cell, preferably a bacterial cell from the phylum Bacteroidetes. The LES can be added to any other protein or polypeptide, more particularly a protein or polypeptide which by nature is not/would not be exported to the cell surface of a Gram-negative bacterial cell.

Preferably the protein or polypeptide has a size of 200 kDa or less, 150 kDa or less, 100 kDa or less, 50 kDa or less, more preferably, 100 kDa or less or 50 kDa or less. Preferably, the protein or polypeptide, which includes fragments of full length proteins comprises at least 5, at least 6, at least 7, at least 8 amino acids, at least 9 amino acids or at least 10 amino acids, preferably at least 10 amino acids residues. Said protein or polypeptide comprising said LES gains the ability to be transported to the Gram-negative bacterial cell surface, preferably a bacterial cell from the phylum Bacteroidetes. Preferably, the LES is inserted at or close to the N-terminus of the polypeptide, more preferably within the first 15 amino acids of the N-terminal region of the mature polypeptide, even more preferably within the first 10 amino acids of the N-terminal region of the mature polypeptide, even more preferably within the first 5 amino acids of the N-terminal region of the mature polypeptide. Most preferably, the LES is located just C-terminally to a cysteine residue. Preferably, said cysteine residue is lipid-modified, more preferably said cysteine residue is the conserved cysteine of the lipobox motif, which originates from the N-terminal signal peptide and typically forms the first amino acid of the mature polypeptide (i.e. “+1 cysteine”) after cleavage of the polypeptide precursor comprising said N-terminal signal peptide by a signal peptidase type II (SPaseII).

In particular embodiments, the invention can be used to expose a polypeptide of Gram-negative bacteria comprising an N-terminal signal peptide but which does not comprise an LES and thus is not surface-exposed. In these embodiments, the LES sequence can be inserted directly adjacent to the C-terminus of said lipobox motif, which, when said lipobox motif consists of the amino acid sequence L(S/A)(A/G)C (SEQ ID NO: 203), is directly adjacent to the cysteine residue thereof.

For certain applications, it might be desirable to remove the LES motif from the polypeptide after surface exposure thereof. For example, removal of the LES motif generates the ‘native’ form of the functionally active polypeptide or fragment thereof. This removal can be achieved by inserting a highly specific protease cleavage site motif between LES motif and the functionally active polypeptide. Preferably, specific cleavage is obtained by use of recombinant endoproteases that recognize a specific sequence (protease/substrate pairs).

The term “protease cleavage site motif” as used herein refers to an amino acid sequence motif cleaved by proteases or chemicals in a given protein. The term “protease”, “peptidase”, or “proteinase” as used herein refers to any enzyme that performs proteolysis, which is the breakdown of proteins into smaller polypeptides or amino acids. In particular embodiments, the amino acid sequence motif is a highly specific protease-sensitive sequence. Non-limiting examples are a tobacco etch virus (TEV) protease cleavage site (ENLYFQIG) (SEQ ID NO: 204) which is specifically cleaved by the TEV protease, Saccharomyces cerevisiae (sc) SUMO (Smt3p) which is specifically cleaved by the scUlp1p protease, Brachypodium distachyon (bd) SUMO which is specifically cleaved by the bdSeNP1 protease, bdNEDD8 which is specifically cleaved by bdNEPD1, Salmo salar (ss) NEDD8 which is specifically cleaved by ssNEDP1, scAtg8 which is specifically cleaved by scAtg4, Xenopus laevis Ub which is specifically cleaved by Usp2, the DDDDK (SEQ ID NO: 205) amino acid motif which is specifically cleaved by E. coli or S. cerevisiae enteropeptidase and the LVPRGS (SEQ ID NO: 206) amino acid motif which is specifically cleaved by Thrombin and Factor Xa. Preferably, the protease includes a tag, which will allow removing the protease from the process by affinity purification. Non-limiting examples of tags are His-tag, FLAG, Streptag II, HA-tag, c-myc and Glutathione S-transferase.

In particular embodiments, the protein or polypeptide is a homologous protein or polypeptide. Expressing proteins at the bacterial surface of a bacterial cell from the phylum Bacteroidetes via the LES according to present invention allows to purify fully functional enzymes from Bacteroidetes, such as glycosylhydrolases or proteases, without the risk of having non-functional or partially functional proteins as it could happen when expressing this type of proteins in other far or non-related bacteria, such as E. coli.

In particular embodiments, the protein or polypeptide is a lipoprotein, such as sialidase (SiaC) or mucinase (MucG), preferably sialidase (SiaC) or mucinase (MucG) of C. canimorsus, even more preferably sialidase (SiaC) or mucinase (MucG) of C. canimorsus 5. In particular embodiments, the protein or polypeptide is a heterologous protein or polypeptide. In particular embodiments, the heterologous protein or polypeptide is a mammalian protein or polypeptide, such as a human protein or polypeptide. In particular embodiments, the heterologous protein or polypeptide is a viral protein or polypeptide or a protein or polypeptide from a bacterial cell which is not of the phylum Bacteroidetes, for example a gram-positive bacterial protein or polypeptide.

The kingdom of Bacteria can be divided into several phyla such as Bacteroidetes. The phylum of Bacteroidetes can be further divided into several classes such as Bacteroidia, Cytophagia, Flavobacteriia, Sphingobacteria and Bacteroidetes incertai sedis. The class of Flavobacteriia can be further divided into families: Cryomorphaceae, Flavobacteriaceae, Myroidaceae and Blattabacteriaceae. The family Flavobacteriaceae includes several genera for example, Flavobacterium, Capnocytophaga, Ornithobacterium and Coenonia. The genus Capnocytophaga can be further divided into species, such as C. canimorsus, C. canis nov. sp., C. cynodegmi, C. gingivalis, C. granulosa, C. haemolytica, C. ochracea and C. sputigena. These scientific classifications are known by the skilled person. The Inventors found that the LES is conserved in the Bacteroidetes phylum. The LES according to present invention is preferably a Bacteroidetes LES, more preferably a C. canimorsus LES, a B. fragilis LES or a Flavobacterium johnsoniae LES, even more preferably a C. canimorsus LES. Furthermore, the Inventors found that there is a shared novel pathway for lipoprotein export in the Bacteroides phylum.

The Inventors discovered that in C. canimorsus surface exposed lipoproteins, a lysine (K) residue followed by either an aspartate (D) or a glutamate (E) residue is conserved in close proximity to the N-terminal cysteine (C) at position +1, more particularly the conserved motif has the following amino acid sequence: CXK(D/E)₂X (SEQ ID NO: 21 to 24), wherein X can by any amino acid. The N-terminal cysteine of said conserved motif is preferably the cysteine of the lipobox motif, which originates from the N-terminal signal peptide and typically forms the first amino acid of the mature polypeptide after cleavage of the polypeptide precursor comprising said N-terminal signal peptide by a signal peptidase type II (SPaseII). Accordingly, the conserved LES motif located just C-terminally to said cysteine residue can have the conserved amino acid motif XK(D/E)₂X (SEQ ID NO:191-194), wherein X can by any amino acid. In particular, the LES consensus motif corresponding to the amino acid sequence QKDDE (SEQ ID NO: 16), has a conservation of 16% (Q), 72% (K), 48% (D), 44% (D) and 23% (E) respectively. The positively charged residue (K) at position +3 is followed by two to three negatively charged amino acids (D and/or E) at positions +4, +5 and +6 immediately after the cysteine residue, preferably a lipidated cysteine residue. The residues at position +2 and +6 downstream of the +1 cysteine are dispensable. The overall charge of the peptide must be negative. The minimal consensus motif corresponds to amino acid sequence KDD, KEE, KDE or KED, preferably KDD, and is sufficient to target lipoproteins to the surface.

For example, within the LES with sequence QKDDE (SEQ ID NO: 16), the least conserved amino acids, namely Q and E, can be substituted by an A, resulting in LES with the following sequences: AKDDE (SEQ ID NO:17) and AKDDA (SEQ ID NO: 18). Also, D can be replaced by E, resulting in LES with the sequence AKEEA (SEQ ID NO: 19) and K can be replaced by A, resulting in LES with the sequence QADDE (SEQ ID NO: 20).

Also, the Inventors discovered that the LES of MucG, which is a naturally surface exposed lipoprotein of C. canimorsus, is KKEVEEE (SEQ ID NO: 49) or part of this sequence, such as KKEVEE (SEQ ID NO: 63), KKEVEEE and KKEVEE both being negatively charged, or KKEVE (SEQ ID NO: 64), which is neutral in charge. The LES of MucG is located directly C-terminally of the +1 cysteine, which is preferably the cysteine of the lipobox motif, which originates from the N-terminal signal peptide and typically forms the first amino acid of the mature polypeptide after cleavage of the polypeptide precursor comprising said N-terminal signal peptide by a signal peptidase type II (SPaseII). Preferably, KKEVEEE (SEQ ID NO: 49) or KKEVEE (SEQ ID NO: 63). Substitutions of one of the K residues of KKEVE (SEQ ID NO: 64) into A, resulting in KAEVE (SEQ ID NO: 65) or AKEVE (SEQ ID NO: 66), can be used to render the LES's overall charge negative. However, the position of the positively charged amino acid, namely K at position +3, is important for proper surface localization. Accordingly, a LES with amino acid sequence AKEVE (SEQ ID NO: 66) is preferred.

Within the LES with sequence KKEVEEE (SEQ ID NO: 49), each individual amino acid can be substituted by an A, resulting in LES with the following sequences: AKEVEEE (SEQ ID NO: 50), KKEAEEE (SEQ ID NO: 51), KKEVEAE (SEQ ID NO: 52), KAEVEEE (SEQ ID NO: 53), KKAVEEE (SEQ ID NO: 54) or KKEVAEE (SEQ ID NO: 55). The following LES sequences are preferred: AKEVEEE (SEQ ID NO: 50), KKEAEEE (SEQ ID NO: 51) or KKEVEAE (SEQ ID NO: 55). Furthermore, one or both lysine in the LES with sequence KKEVEEE (SEQ ID NO: 49) can be substituted by R, resulting in LES with the following sequences: RREVEEE (SEQ ID NO: 60), RAEVEEE (SEQ ID NO: 61) or AREVEEE (SEQ ID NO: 62), preferably RAEVEEE (SEQ ID NO: 61) or AREVEEE (SEQ ID NO: 62), more preferably RAEVEEE (SEQ ID NO: 61).

Within the LES, an S at position +2 or a K at position +3, or an amino acid with a positive charge at position +2 or +3, is required for surface export. The minimal LES for optimal MucG surface exposure is XK(D/E)₃ (SEQ ID NO: 40 to 47) downstream from the +1 C, preferably a lipid-modified C, wherein X can be any amino acid.

Furthermore, the Inventors discovered that B. fragilis surface exposed lipoproteins have an N-terminal negatively charged consensus sequence in close proximity to the +1 cysteine, preferably said cysteine is lipid-modified, more particularly a consensus sequence with the amino acid sequence SDDDD (SEQ ID NO: 1). Also, the Inventors discovered that F. johnsoniae surface exposed lipoproteins have an N-terminal consensus sequence with the amino acid sequence SDDFE (SEQ ID NO: 2). Amino acid D and E, and S and T, are interchangeable within SEQ ID NO: 1 and SEQ ID NO: 2. Accordingly, the LES can comprise any one of SEQ ID NO: 3 to SEQ ID NO: 15 or SEQ ID NO: 25 to SEQ ID NO: 39. As long as the overall charge of the peptide is negative.

The LES of C. canimorsus, B. fragilis and F. johnsoniae share a positively charged or polar residue followed by 2 or 3 negatively charged residues, giving an overall negative charge in close proximity to the +1 cysteine. The skilled person will understand that the LES according to present invention can be any Bacteroidetes LES which complies with these properties. Accordingly, the LES of the invention comprises an amino acid sequence according to any one of the following consensus sequences X₁X₂DD (SEQ ID NO: 68), X₁X₂DE (SEQ ID NO: 69), X₁X₂ED (SEQ ID NO: 70) or X₁X₂EE (SEQ ID NO: 71), wherein X₁ can be any amino acid and X₂ is selected from the group consisting of K, S, T and A, with the proviso that when X₂ is A, X₁ is Q.

Alternatively, the LES of the invention comprises an amino acid sequence according to any one of the following consensus sequence:
XJZZ (SEQ ID NO: 197), wherein X can be any amino acid, wherein J is selected from the group consisting of K and A, wherein Z is selected from the group consisting of D and E, with the proviso that when J is A, X is Q;
BZZUZ (SEQ ID NO: 198), wherein B is selected from the group consisting of S and T, wherein Z is selected from the group consisting of D and E and wherein U is selected from the group consisting of D, E and F; or
XKEOE (SEQ ID NO: 199), preferably XKEOEE (SEQ ID NO: 200), wherein X and O can be any amino acid, preferably wherein O is V;

preferably XJZZ, wherein X can be any amino acid, wherein J is selected from the group consisting of K and A, wherein Z is selected from the group consisting of D and E; with the proviso that when J is A, X is Q.

In particular embodiments, said lipoprotein export signal is overall negatively charged.

In particular embodiments, said LES is KDD, KDE, KEE, or any of the sequences as set forth in SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, more preferably any of the sequences as set forth in SEQ ID NO: 1, 2, 3, 4, 5, 6, 7, 8, 9, 10, 11, 12, 13, 14, 15, 16, 17, 18, 25, 26, 27, 28, 29, 30, 31, 32, 33, 34, 35, 46, 37, 38, 39, 40, 41, 42, 43, 44, 45, 46 or 47, even more preferably, any of the sequences as set forth in SEQ ID NO: 1, 2, 16, 17 or 18.

In particular embodiments, said LES is any of the sequences as set forth in
SEQ ID NO: 16, 17, 18, 19, 20, 40, 41, 42, 43, 44, 45, 46, 47, 191, 192, 193 or 194, preferably SEQ ID NO: 16, 17, 18, 19, 20, 40, 41, 42, 43, 44, 45, 46 or 47;
SEQ ID NO: 1, 2, 3, 4, 5, 6, 7, 8, 9, 10, 11, 12, 13, 14, 15, 25, 26, 27, 28, 29, 30, 31, 32, 33, 34, 35, 36, 37, 38 or 39; or
SEQ ID NO: 49, 50, 51, 63, 64 or 66, preferably SEQ ID NO: 49, 50, 51 or 63. In particular embodiments, said LES is any of the sequences as set forth in SEQ ID NO: 16, 17, 18, 19, 20, 40, 41, 42, 43, 44, 45, 46, 47, 191, 192, 193 or 194, preferably SEQ ID NO: 16, 17, 18, 19, 20, 40, 41, 42, 43, 44, 45, 46 or 47.

TABLE 1
a list of non-limiting examples of LES.

| SEQ ID NO. | type | sequence |
|---|---|---|
| 1 | amino acid | SDDDD |
| 2 | amino acid | SDDFE |
| 3 | amino acid | SEEEE |
| 4 | amino acid | SDEDE |
| 5 | amino acid | SEDED |
| 6 | amino acid | SDDEE |
| 7 | amino acid | SEEDD |
| 8 | amino acid | SEEED |
| 9 | amino acid | SDDDE |
| 10 | amino acid | SEEFE |
| 11 | amino acid | SEEFD |
| 12 | amino acid | SEDFE |
| 13 | amino acid | SDEFE |
| 14 | amino acid | SEDFD |
| 15 | amino acid | SDEFD |
| 16 | amino acid | QKDDE |
| 17 | amino acid | AKDDE |
| 18 | amino acid | AKDDA |
| 19 | amino acid | AKEEA |
| 20 | amino acid | QADDE |
| 21 | amino acid | CXKDEX* |
| 22 | amino acid | CXKEDX* |
| 23 | amino acid | CXKDDX* |
| 24 | amino acid | CXKEEX* |
| 25 | amino acid | TDDDD |
| 26 | amino acid | TDDFE |
| 27 | amino acid | TEEEE |
| 28 | amino acid | TDEDE |
| 29 | amino acid | TEDED |
| 30 | amino acid | TDDEE |
| 31 | amino acid | TEEDD |
| 32 | amino acid | TEEED |
| 33 | amino acid | TDDDE |
| 34 | amino acid | TEEFE |
| 35 | amino acid | TEEFD |
| 36 | amino acid | TEDFE |
| 37 | amino acid | TDEFE |
| 38 | amino acid | TEDFD |
| 39 | amino acid | TDEFD |
| 40 | amino acid | XKDDD* |
| 41 | amino acid | XKEEE* |
| 42 | amino acid | XKDDE* |
| 43 | amino acid | XKDED* |
| 44 | amino acid | XKDEE* |
| 45 | amino acid | XKEDE* |
| 46 | amino acid | XKEDD* |
| 47 | amino acid | XKEED* |
| 48 | amino acid | CKKEVEVEEE |
| 49 | amino acid | KKEVEEE |
| 50 | amino acid | AKEVEEE |
| 51 | amino acid | KKEAEEE |
| 52 | amino acid | KKEVEAE |
| 53 | amino acid | KAEVEEE |
| 54 | amino acid | KKAVEEE |
| 55 | amino acid | KKEVAEE |
| 56 | amino acid | AAEVEEE |
| 57 | amino acid | KKAAAAA |
| 58 | amino acid | KKAAAEE |
| 59 | amino acid | KKEVAAA |
| 60 | amino acid | RREVEEE |
| 61 | amino acid | RAEVEEE |
| 62 | amino acid | AREVEEE |
| 63 | amino acid | KKEVEE |
| 64 | amino acid | KKEVE |
| 65 | amino acid | KAEVE |
| 66 | amino acid | AKEVE |
| 67 | amino acid | KEVEE |
| 68 | amino acid | X₁X₂DD*** |
| 69 | amino acid | X₁X₂DE*** |
| 70 | amino acid | X₁X₂ED*** |
| 71 | amino acid | X₁X₂EE*** |
| 191 | amino acid | XKDEX* |
| 192 | amino acid | XKEDX* |
| 193 | amino acid | XKDDX* |
| 194 | amino acid | XKEEX* |
| 197 | amino acid | XJZZ** |
| 198 | amino acid | BZZUZ* |
| 199 | amino acid | XKEOE* |
| 200 | amino acid | XKEOEE* |
| 201 | amino acid | XKEVE* |
| 202 | amino acid | XKEVEE* |

*wherein X can be any amino acid, wherein B is selected from the group consisting of S and T, wherein Z is selected from the group consisting of D and E, wherein U is selected from the group consisting of D, E and F, wherein O can be any amino acid, preferably wherein O is V
** wherein X can be any amino acid, wherein J is selected from the group consisting of K and A, wherein Z is selected from the group consisting of D and E; with the proviso that when J is A, X is Q
***wherein X₁ can be any amino acid and X₂ is selected from the group consisting of K, S, T and A, with the proviso that when X₂ is A, X₁ is Q.

Successful surface-exposure of the polypeptide comprising the LES according to the invention can be verified by use of several experiments including membrane protein fractionation, fluorescence or confocal microscopy, fluorescence-based flow cytometry, ELISA and, if the polypeptide is an enzyme, by activity assay.

In particular embodiments, the polypeptide comprising the LES comprises the amino acid sequence KDD or XKDDX (SEQ ID NO: 70), preferably XKDDX, wherein X can be any amino acid residue.

In particular embodiments, the polypeptide comprising the LES according to present invention comprises one cysteine residue at an amino acid position +1 from the N-terminus of the amino acid sequence as set forth any one of the consensus sequences according to the invention, preferably, wherein said cysteine residue is lipid-modified, more preferably wherein said cysteine residue originates from an N-terminal signal peptide.

The polypeptide of interest can be fused to the LES by N-terminal fusion.

In order to be efficiently transported from the cytosol to the bacterial cell surface, the recombinant polypeptide requires at least one specific signal peptide in addition to the LES motif. More particularly, a classical lipoprotein signal peptide comprising a lipobox motif which is specifically recognized by a SPaseII is required to translocate the polypeptide from the cytosol to the periplasm of the bacterial cell. Accordingly, since the signal peptide is cleaved off once the polypeptide has reached the periplasm of the bacterial cell, only the polypeptide precursor and not the final functionally active polypeptide, will comprise the full signal peptide sequence.

Accordingly, another aspect of the invention is a polypeptide precursor comprising

(a) an N-terminal signal peptide wherein said signal peptide preferably comprises a lipobox motif which is specifically recognized by a signal peptidase type II,

(b) a LES comprising the amino acid sequence according to any one of the following consensus sequences: X₁X₂DD (SEQ ID NO: 68), X₁X₂DE (SEQ ID NO: 69), X₁X₂ED (SEQ ID NO: 70) or X₁X₂EE (SEQ ID NO: 71), wherein X₁ can be any amino acid and X₂ is selected from the group consisting of K, S, T and A, with the proviso that when X₂ is A, X₁ is Q, wherein said lipoprotein export signal is located C-terminally of said signal peptide;

(c) optionally, a protease cleavage site motif, wherein said protease cleavage site motif is different from said lipobox motif and is located C-terminally of said signal peptide and said LES; and

(d) a polypeptide.

The term “polypeptide precursor” or “pro-polypeptide” as used herein, refers to a primary translation product of the mRNA encoding for a polypeptide comprising a LES according to the invention. Said polypeptide precursor comprises a short N-terminal signal peptide, which is needed to target the polypeptide precursor to a certain location. Once the polypeptide precursor has reached its location, the signal peptide is cleaved off, resulting in the polypeptide. Preferably, said location is the inner membrane or periplasmic space of a gram-negative bacterial cell.

The term “N-terminal signal peptide” as used herein refers to a lipoprotein signal peptide which is recognized and cleaved by the SPaseII, is located at the N-terminus of the polypeptide, more particularly the lipoprotein, and is required for the export of the polypeptide, more particularly the lipoprotein, from the cytosol across the inner membrane of a Gram-negative bacterial cell. The C-terminus of the lipoprotein signal peptide contains a four-amino-acid motif, called the “lipobox”. Preferably, the N-terminal signal peptide consists of at least 16 amino acid residues and at most 35 amino acid residues. The skilled person will understand that the N-terminal signal peptide can be any lipoprotein signal peptide comprising a lipobox motif which is recognized and cleaved by SPase II. Non-limiting examples of such N-terminal signal peptides can be the signal peptide of sialidase (siaC) of C. canimorsus 5 having the amino acid sequence MNRIFYLLFAFVLLSACGS (SEQ ID NO: 195) or mucinase (MucG) having the amino acid sequence MKKIVSISLFFLISATIWLACK (SEQ ID NO: 196). The term “lipobox motif” as used herein refers to an amino acid sequence motif which is recognized first by the prolipoprotein diacylglycerol transferase that attaches a diacylglycerol moiety derived from membrane phosphatidylglycerol, to the SH of the +1 cysteine. Then the lipobox is recognized by SPase II that cleaves the signal peptide from the prolipoprotein. Following signal peptide cleavage, the cysteine forming the N-terminus of the mature protein is modified with an additional acyl chain, extracted from the inner membrane and transported across the periplasm by the Lol system and subsequently inserted into the OM (FIG. 13). The lipobox motif is typically a four-amino-acid motif which has a conserved lipid-modified cysteine residue, more particularly a cysteine residue to which a glyceride-fatty acid lipid is attached, that allows the lipoprotein to anchor onto the periplasmic leaflet of the plasma membrane or outer membrane. More particularly, the conserved cysteine is located at position +1 and has a G or A at position −1, an A or S at position −2 and an L at position −3. Cleavage of the prolipoprotein by SPaseII occurs N terminally of the +1 position cysteine residue, i.e., within the lipobox.

Another aspect relates to a polypeptide precursor comprising

(a) an N-terminal signal peptide of a lipoprotein of Gram-negative bacteria comprising a lipobox motif located at the very end of the C-terminus of said signal peptide, wherein said lipobox motif consists of the amino acid sequence L(S/A)(A/G)C (SEQ ID NO: 203) and is specifically recognizable by a signal peptidase type II;

(b) a lipoprotein export signal comprising an amino acid sequence according to any one of the following consensus sequences:
XJZZ (SEQ ID NO: 197), wherein X can be any amino acid, wherein J is selected from the group consisting of K and A, wherein Z is selected from the group consisting of D and E, with the proviso that when J is A, X is Q;
BZZUZ (SEQ ID NO: 198), wherein B is selected from the group consisting of S and T, wherein Z is selected from the group consisting of D and E and wherein U is selected from the group consisting of D, E and F; or
XKEOE (SEQ ID NO: 199), preferably XKEOEE (SEQ ID NO: 200), wherein X and O can be any amino acid, preferably wherein O is V;

preferably XJZZ, wherein X can be any amino acid, wherein J is selected from the group consisting of K and A, wherein Z is selected from the group consisting of D and E; with the proviso that when J is A, X is Q;

wherein said lipoprotein export signal is located directly adjacent to the C-terminus of said signal peptide;

(c) a polypeptide, wherein said polypeptide is located C-terminally of said signal peptide and said lipoprotein export signal; and

(d) optionally, a protease cleavage site motif, wherein said protease cleavage site motif is different from said lipobox motif and is located C-terminally of said signal peptide and said lipoprotein export signal and N-terminally of said polypeptide.

In particular embodiments, said lipoprotein export signal is overall negatively charged.

In particular embodiments, said signal peptide, said lipoprotein export signal and said polypeptide, do not naturally occur together in a polypeptide sequence.

For clarity purposes, the representation of the lipobox motif having amino acid sequence L(S/A)(A/G)C (SEQ ID NO: 203) may also be referred to herein as amino acid sequence LX₃X₄C, wherein “X₃” can be amino acid S or A and wherein “X₄” can be amino acid A or G.

In particular embodiments, said LES is any of the sequences as set forth in SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, preferably any of the sequences as set forth in SEQ ID NO: 1, 2, 3, 4, 5, 6, 7, 8, 9, 10, 11, 12, 13, 14, 15, 16, 17, 18, 25, 26, 27, 28, 29, 30, 31, 32, 33, 34, 35, 46, 37, 38, 39, 40, 41, 42, 43, 44, 45, 46 or 47, more preferably, any of the sequences as set forth in SEQ ID NO: 1, 2, 16, 17 or 18.

In particular embodiments, said LES present in the polypeptide precursor is any of the sequences as set forth in
SEQ ID NO: 16, 17, 18, 19, 20, 40, 41, 42, 43, 44, 45, 46, 47, 191, 192, 193 or 194, preferably SEQ ID NO: 16, 17, 18, 19, 20, 40, 41, 42, 43, 44, 45, 46 or 47;
SEQ ID NO: 1, 2, 3, 4, 5, 6, 7, 8, 9, 10, 11, 12, 13, 14, 15, 25, 26, 27, 28, 29, 30, 31, 32, 33, 34, 35, 36, 37, 38 or 39; or
SEQ ID NO: 49, 50, 51, 63, 64 or 66, preferably SEQ ID NO: 49, 50, 51 or 63.

In preferred embodiments, said LES present in the polypeptide precursor is any of the sequences as set forth in SEQ ID NO: 16, 17, 18, 19, 20, 40, 41, 42, 43, 44, 45, 46, 47, 191, 192, 193 or 194, preferably SEQ ID NO: 16, 17, 18, 19, 20, 40, 41, 42, 43, 44, 45, 46 or 47.

In particular embodiments, said N-terminal signal peptide present in the polypeptide precursor is a Bacteroidetes N-terminal signal peptide, more preferably a C. canimorsus N-terminal signal peptide, a B. fragilis N-terminal signal peptide or a Flavobacterium johnsoniae N-terminal signal peptide, even more preferably a C. canimorsus N-terminal signal peptide.

In particular embodiments, said N-terminal signal peptide is the signal peptide of sialidase (siaC) or mucinase (MucG), preferably sialidase (siaC) or mucinase (MucG) of C. canimorsus, even more preferably sialidase (SiaC) or mucinase (MucG) of C. canimorsus 5.

In particular embodiments, said N-terminal signal peptide is the signal peptide of sialidase (siaC) of C. canimorsus 5 having the amino acid sequence MNRIFYLLFAFVLLSACGS (SEQ ID NO: 195) or the signal peptide of mucinase (MucG) of C. canimorsus 5 having the amino acid sequence MKKIVSISLFFLISATIWLACK (SEQ ID NO: 196).

Another aspect of the invention is a nucleic acid encoding the polypeptide or the polypeptide precursor according to the invention.

By “nucleic acid” is meant oligomers and polymers of any length composed essentially of nucleotides, e.g., deoxyribonucleotides and/or ribonucleotides. Nucleic acids can comprise purine and/or pyrimidine bases and/or other natural (e.g., xanthine, inosine, hypoxanthine), chemically or biochemically modified (e.g., methylated), non-natural, or derivatised nucleotide bases. The backbone of nucleic acids can comprise sugars and phosphate groups, as can typically be found in RNA or DNA, and/or one or more modified or substituted sugars and/or one or more modified or substituted phosphate groups. Modifications of phosphate groups or sugars may be introduced to improve stability, resistance to enzymatic degradation, or some other useful property. A “nucleic acid” can be for example double-stranded, partly double stranded, or single-stranded. Where single-stranded, the nucleic acid can be the sense strand or the antisense strand. In addition, nucleic acid can be circular or linear. The term “nucleic acid” as used herein preferably encompasses DNA and RNA, specifically including RNA, genomic RNA, cDNA, DNA, provirus, pre-mRNA and mRNA.

The nucleic acid according to present invention can be comprised in a nucleic acid construct, operably linked to one or more control sequences capable of directing the expression of the polypeptide in a suitable expression host. The term nucleic acid construct refers to an artificially constructed segment of nucleic acid which is going to be transferred into an expression host. An operable linkage is a linkage in which regulatory sequences and sequences sought to be expressed are connected in such a way as to permit said expression. For example, sequences, such as, e.g., a promoter and an ORF, may be said to be operably linked if the nature of the linkage between said sequences does not: (1) result in the introduction of a frame-shift mutation, (2) interfere with the ability of the promoter to direct the transcription of the ORF, (3) interfere with the ability of the ORF to be transcribed from the promoter sequence. Hence, “operably linked” may mean incorporated into a genetic construct so that expression control sequences, such as a promoter, effectively control expression of a coding sequence of interest, such as the nucleic acid molecule as defined herein.

The nucleic acid sequence can also encompass a nucleic acid fragment encoding a tag. Tags can be used for various purposes, such as purification of the expressed peptide (e.g poly (His) tag), to assist proper protein folding (e.g. thioredoxin), separation techniques (e.g. FLAG-tag), or enzymatic or chemical modifications (e.g. biotin ligase tags, FlAsh), or detection (e.g. AviTag, Calmodulin-tag, polyglutamate tag, E-tag, FLAG-tag, HA-tag, His-tag, Myc-tag, S-tag, SBP-tag, Softag 1, Softag 3, Strep tag, TC tag, V5 tag, VSV-tag, Xpress tag, Isopeptag, SpyTag, Biotin Carboxyl Carrier Protein, Glutathione-S-transferase-tag, Green fluorescent protein tag, Halo-tag, Maltose binding protein-tag, Nus-tag, Thioredoxin-tag or Fc-tag). In the context of the present invention, their main purpose is purification.

Another aspect according to the invention relates to a recombinant expression vector comprising the nucleic acid according to the invention, a promoter, and transcriptional, translational stop signals, and preferably, a selectable marker.

The term “vector” as used herein, is a tool that allows or facilitates the transfer of an entity from one environment to another. It is a replicon, such as a plasmid, phage, or cosmid, into which another DNA segment may be inserted so as to bring about the replication of the inserted segment. In present application, a vector is a nucleic acid molecule capable of transporting another nucleic acid to which it has been linked. One type of vector is a “plasmid” which refers to a circular double stranded DNA loop into which additional DNA segments can be ligated. Another type of vector is a phage vector. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “recombinant vectors”). In general, expression vectors of utility in recombinant DNA techniques are often in the form of plasmids. In the present specification, “plasmid” and “vector” can be used interchangeably as the plasmid is the most commonly used form of vector.

Factors of importance in selecting a particular vector include inter alia: choice of recipient host cell, ease with which recipient cells that contain the vector may be recognised and selected from those recipient cells which do not contain the vector; the number of copies of the vector which are desired in particular recipient cells; whether it is desired for the vector to integrate into the chromosome or to remain extra-chromosomal in the recipient cells; and whether it is desirable to be able to “shuttle” the vector between recipient cells of different species.

Expression vectors can be autonomous or integrative. A recombinant nucleic acid can be in introduced into the host cell in the form of an expression vector such as a plasmid, phage, transposon, cosmid or virus particle. The recombinant nucleic acid can be maintained extrachromosomally or it can be integrated into the cell chromosomal DNA. Expression vectors can contain selection marker genes encoding proteins required for cell viability under selected conditions (e.g., URA3, which encodes an enzyme necessary for uracil biosynthesis or TRP1, which encodes an enzyme required for tryptophan biosynthesis) to permit detection and/or selection of those cells transformed with the desired nucleic acids. Expression vectors can also include an autonomous replication sequence (ARS).

Integrative vectors generally include a serially arranged sequence of at least a first insertable DNA fragment, a selectable marker gene, and a second insertable DNA fragment. The first and second insertable DNA fragments are each about 200 (e.g., about 250, about 300, about 350, about 400, about 450, about 500, or about 1000 or more) nucleotides in length and have nucleotide sequences which are homologous to portions of the genomic DNA of the host cell species to be transformed. A nucleotide sequence containing a gene of interest for expression is inserted in this vector between the first and second insertable DNA fragments, whether before or after the marker gene. Integrative vectors can be linearized prior to transformation to facilitate the integration of the nucleotide sequence of interest into the host cell genome.

A vector can be introduced into a host cell using a variety of methods. Methods of transfection foreign DNA into a host cell are known in the art and can involve instruments (e.g. electroporation, biolistic technology, microinjection, laserfection, opto-injection) or reagents (e.g. lipids, calcium phosphate, cationic polymers, DEAE-dextran, activated dendrimers or magnetic beads), can be virus-mediated or by any other means known by the skilled person. In stable transfections, cells have integrated the foreign DNA in their genome. In transient transfections, the foreign DNA does not integrate in the genome but genes are expressed for a limited time (24-96 h). The term “transformation” is used to describe foreign DNA transfer in bacteria and non-animal eukaryotic cells. This can be obtained by heat-shock of chemically competent bacteria, by electroporation or other methods of transformation known in the art.

The term “host cell” as used herein, refers to the cell that has been introduced with one or more polynucleotides, preferably DNA, by transfection. By means of an example, the host cell may be a bacterial cell, a fungal cell, including yeast cells, an animal cell, or a mammalian cell, including human cells and non-human mammalian cells. Preferably, bacterial cells from a species that can be used in a biosafety level (BSL) 1 or 2 (BSLs for bacteria are determined by, for example, U.S. Public Health Service guidelines or in the Council Directive 90/679/EEC of 26 Nov. 1990 on the protection of workers from risks related to exposure to biological agents at work, OJ No. L 374, p. 1.), more preferably a bacterial cell of the Bacteroidetes phylum, even more preferably Capnocytophaga canimorsus or Flavobacterium johnsoniae, most preferably Capnocytophaga canimorsus.

As used herein, the term “promoter” refers to a DNA sequence that enables a gene to be transcribed. A promoter is recognized by RNA polymerase, which then initiates transcription. Thus, a promoter contains a DNA sequence that is either bound directly by, or is involved in the recruitment, of RNA polymerase. A promoter sequence can also include “enhancer regions”, which are one or more regions of DNA that can be bound with proteins (namely the trans-acting factors) to enhance transcription levels of genes in a gene-cluster. The enhancer, while typically at the 5′ end of a coding region, can also be separate from a promoter sequence, e.g., can be within an intronic region of a gene or 3′ to the coding region of the gene.

The promotor may be a constitutive or inducible (conditional) promoter. A constitutive promoter is understood to be a promoter whose expression is constant under the standard culturing conditions. Inducible promoters are promoters that are responsive to one or more induction cues. For example, an inducible promoter can be chemically regulated (e.g., a promoter whose transcriptional activity is regulated by the presence or absence of a chemical inducing agent such as an alcohol, tetracycline, a steroid, a metal, or other small molecule) or physically regulated (e.g., a promoter whose transcriptional activity is regulated by the presence or absence of a physical inducer such as light or high or low temperatures). An inducible promoter can also be indirectly regulated by one or more transcription factors that are themselves directly regulated by chemical or physical cues.

As used herein, the term “stop signal” refers to a transcription terminator or a translational stop codon. A transcription terminator is a fragment of nucleic acid sequence that indicates the end of a gene or operon in genomic DNA during transcription. This sequence provides signals in the newly synthesized mRNA that trigger processes which release the mRNA from the transcriptional complex, thereby mediating transcriptional termination. A stop codon is a nucleotide triplet within mRNA that does not code for an amino acid and thereby signals the termination of the synthesis of a protein. In RNA, this stop codon can be UAG, UAA or UGA, wherein U is uracil, A is adenine and G is guanine.

As used herein, the term “selectable marker” refers to a marker gene, such that it can be determined whether or not the cell is capable of expressing the different nucleic acids of the nucleic acid construct based on the expression of this marker gene. Typically marker genes are used that confer resistance to a compound, which is added to the culture medium of the host cell, and will eliminate untransfected cells but not the transfected cells (positive selection, e.g. resistance to antibiotics). For example, selection antibiotics can be geneticin, zeocin, hygromycin B, puromycin, erythromycin, cefoxitin, gentamicin or blasticidin. Their coding sequences are typically incorporated into the nucleic acid vector used for delivering genetic material into a target cell.

Furthermore, the invention also relates to a recombinant expression vector comprising

(a) a nucleic acid sequence encoding a LES comprising the amino acid sequence according to any one of the following consensus sequences: X₁X₂DD (SEQ ID NO: 68), X₁X₂DE (SEQ ID NO: 69), X₁X₂ED (SEQ ID NO: 70) or X₁X₂EE (SEQ ID NO: 71), wherein X₁ can be any amino acid and X₂ is selected from the group consisting of K, S, T and A, with the proviso that when X₂ is A, X₁ is Q;

(b) optionally, a nucleic acid sequence encoding a signal peptide wherein said signal peptide preferably comprises a lipobox motif which is specifically recognized by a signal peptidase type II, and wherein said nucleic acid sequence encoding said signal peptide is located 5′ of said nucleic acid sequence encoding said LES;

(c) optionally, a nucleic acid sequence encoding a protease cleavage site motif, wherein said nucleic acid sequence encoding said protease cleavage site motif is different from said nucleic acid sequence encoding said lipobox motif and is located 3′ of said nucleic acid sequence encoding said LES; and

(d) a multiple cloning site, wherein said multiple cloning site is located 3′ of said nucleic acid encoding said LES and said protease cleavage site motif.

The term “multiple cloning site” as used herein refers to short segment of DNA which contains multiple, preferably 5, 10, 15 or 20, restriction enzyme recognition sites in close proximity of each other, wherein said restriction enzyme recognition sites typically occur only once within a vector comprising said multiple cloning site. Accordingly, when a restriction enzyme cleaves one of said restriction enzyme recognition sites, the vector is linearised, but not fragmented.

The invention also relates to a recombinant expression vector comprising

(a) a nucleic acid sequence encoding a signal peptide of a lipoprotein of Gram-negative bacteria wherein said signal peptide comprises a lipobox motif located at the very end of the C-terminus of said signal peptide, wherein said lipobox motif consists of the amino acid sequence L(S/A)(A/G)C (SEQ ID NO: 203) and is specifically recognized by a signal peptidase type II;

(b) a nucleic acid sequence encoding a lipoprotein export signal having an amino acid sequence according to any one of the following consensus sequences:
XJZZ (SEQ ID NO: 197), wherein X can be any amino acid, wherein J is selected from the group consisting of K and A, wherein Z is selected from the group consisting of D and E; with the proviso that when J is A, X is Q;
BZZUZ (SEQ ID NO: 198), wherein B is selected from the group consisting of S and T, wherein Z is selected from the group consisting of D and E and wherein U is selected from the group consisting of D, E and F; or
XKEOE (SEQ ID NO: 199), preferably XKEOEE (SEQ ID NO: 200), wherein X and O can be any amino acid, preferably wherein O is V;

preferably XJZZ, wherein X can be any amino acid, wherein J is selected from the group consisting of K and A, wherein Z is selected from the group consisting of D and E; with the proviso that when J is A, X is Q;

wherein said nucleic acid sequence encoding said lipoprotein export signal is located directly downstream of said nucleic acid sequence encoding said signal peptide;

(c) optionally, a nucleic acid sequence encoding a protease cleavage site motif, wherein said protease cleavage site motif is different from said lipobox motif and is located downstream of said nucleic acid sequence encoding said lipoprotein export signal and said nucleic acid sequence encoding said signal peptide; and

(d) a multiple cloning site, wherein said multiple cloning site is located downstream of said nucleic acid encoding said lipoprotein export signal and said nucleic acid encoding said signal peptide and, optionally downstream of said protease cleavage site motif.

In particular embodiments, said lipoprotein export signal is overall negatively charged.

In particular embodiments, said LES is any of the sequences as set forth in SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, preferably any of the sequences as set forth in SEQ ID NO: 1, 2, 3, 4, 5, 6, 7, 8, 9, 10, 11, 12, 13, 14, 15, 16, 17, 18, 25, 26, 27, 28, 29, 30, 31, 32, 33, 34, 35, 46, 37, 38, 39, 40, 41, 42, 43, 44, 45, 46 or 47, more preferably, any of the sequences as set forth in SEQ ID NO: 1, 2, 16, 17 or 18.

In particular embodiments, said LES is any of the sequences as set forth in
SEQ ID NO: 16, 17, 18, 19, 20, 40, 41, 42, 43, 44, 45, 46, 47, 191, 192, 193 or 194, preferably SEQ ID NO: 16, 17, 18, 19, 20, 40, 41, 42, 43, 44, 45, 46, 47;
SEQ ID NO: 1, 2, 3, 4, 5, 6, 7, 8, 9, 10, 11, 12, 13, 14, 15, 25, 26, 27, 28, 29, 30, 31, 32, 33, 34, 35, 36, 37, 38 or 39; or
SEQ ID NO: 49, 50, 51, 63, 64 or 66, preferably SEQ ID NO: 49, 50, 51 or 63.

In preferred embodiments, said LES is any of the sequences as set forth in SEQ ID NO: 16, 17, 18, 19, 20, 40, 41, 42, 43, 44, 45, 46, 47, 191, 192, 193 or 194, preferably SEQ ID NO: 16, 17, 18, 19, 20, 40, 41, 42, 43, 44, 45, 46 or 47.

In particular embodiments, said N-terminal signal peptide is a Bacteroidetes N-terminal signal peptide, more preferably a C. canimorsus N-terminal signal peptide, a B. fragilis N-terminal signal peptide or a Flavobacterium johnsoniae N-terminal signal peptide, even more preferably a C. canimorsus N-terminal signal peptide.

In particular embodiments, said N-terminal signal peptide is the signal peptide of sialidase (siaC) or mucinase (MucG) of C. canimorsus, even more preferably sialidase (SiaC) or mucinase (MucG) of C. canimorsus 5.

In particular embodiments, said N-terminal signal peptide is the signal peptide of sialidase (siaC) of C. canimorsus 5 having the amino acid sequence MNRIFYLLFAFVLLSACGS (SEQ ID NO: 195) or the signal peptide of mucinase (MucG) of C. canimorsus 5 having the amino acid sequence MKKIVSISLFFLISATIWLACK (SEQ ID NO: 196).

Bacterial host cells may be bacterial cells from all bacterial species as known by the one skilled in the art. Preferably, bacterial species that can be used in a biosafety level (BSL) 1 or 2 (BSLs for bacteria are determined by, for example, U.S. Public Health Service guidelines or in the Council Directive 90/679/EEC of 26 Nov. 1990 on the protection of workers from risks related to exposure to biological agents at work, OJ No. L 374, p. 1.)

In particular embodiments, the host cell according to the invention is a bacterial cell, preferably bacterial cell of the Bacteroides phylum, more preferably Capnocytophaga canimorsus or Flavobacterium johnsoniae, even more preferably Capnocytophaga canimorsus.

The invention also provides the use of a LES comprising an amino acid sequence according to one of the following consensus sequences: X₁X₂DD (SEQ ID NO: 68), X₁X₂DE (SEQ ID NO: 69), X₁X₂ED (SEQ ID NO: 70) or X₁X₂EE (SEQ ID NO: 71), wherein X₁ can be any amino acid and X₂ is selected from the group consisting of K, S, T and A, wherein X₂ can only be A if X₁ is Q, for surface exposure of a polypeptide such as a functionally active polypeptide in a host cell, wherein said polypeptide originates from the same or a different organism than said host cell.

Furthermore, the invention also provides the use of a lipoprotein export signal comprising an amino acid sequence according to one of the following consensus sequences:
XJZZ (SEQ ID NO: 197), wherein X can be any amino acid, wherein J is selected from the group consisting of K and A, wherein Z is selected from the group consisting of D and E; with the proviso that when J is A, X is Q;
BZZUZ (SEQ ID NO: 198), wherein B is selected from the group consisting of S and T, wherein Z is selected from the group consisting of D and E and wherein U is selected from the group consisting of D, E and F; or
XKEOE (SEQ ID NO: 199), preferably XKEOEE (SEQ ID NO: 200), wherein X and O can be any amino acid, preferably wherein O is V;

preferably XJZZ, wherein X can be any amino acid, wherein J is selected from the group consisting of K and A, wherein Z is selected from the group consisting of D and E; with the proviso that when J is A, X is Q;

for surface exposure of a polypeptide in a host cell, wherein said polypeptide originates from the same or a different organism than said host cell.

In particular embodiments, said lipoprotein export signal is overall negatively charged.

In particular embodiments, said lipoprotein export signal is located directly adjacent to an N-terminal lipid-modified cysteine residue originating from an N-terminal signal peptide of a lipoprotein of Gram-negative bacteria comprising a lipobox motif located at the very end of the C-terminus of said signal peptide, wherein said lipobox motif consists of the amino acid sequence L(S/A)(A/G)C (SEQ ID NO: 230) and is specifically recognizable by a signal peptidase type II.

In particular embodiments, said lipoprotein export signal and said polypeptide do not naturally occur together in a polypeptide sequence.

In particular embodiments, said LES is any of the sequences as set forth in SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, preferably any of the sequences as set forth in SEQ ID NO: 1, 2, 3, 4, 5, 6, 7, 8, 9, 10, 11, 12, 13, 14, 15, 16, 17, 18, 25, 26, 27, 28, 29, 30, 31, 32, 33, 34, 35, 46, 37, 38, 39, 40, 41, 42, 43, 44, 45, 46 or 47, more preferably, any of the sequences as set forth in SEQ ID NO: 1, 2, 16, 17 or 18.

In particular embodiments, said LES is any of the sequences as set forth in
SEQ ID NO: 16, 17, 18, 19, 20, 40, 41, 42, 43, 44, 45, 46, 47, 191, 192, 193 or 194, preferably SEQ ID NO: 16, 17, 18, 19, 20, 40, 41, 42, 43, 44, 45, 46 or 47;
SEQ ID NO:1, 2, 3, 4, 5, 6, 7, 8, 9, 10, 11, 12, 13, 14, 15, 25, 26, 27, 28, 29, 30, 31, 32, 33, 34, 35, 36, 37, 38 or 39; or
SEQ ID NO: 49, 50, 51, 63, 64 or 66, preferably SEQ ID NO:49, 50, 51 or 63.

In preferred embodiments, said LES is any of the sequences as set forth in SEQ ID NO: 16, 17, 18, 19, 20, 40, 41, 42, 43, 44, 45, 46, 47, 191, 192, 193 or 194, preferably SEQ ID NO: 16, 17, 18, 19, 20, 40, 41, 42, 43, 44, 45, 46 or 47.

Many diseases which previously contributed to mortality are now prevented by vaccination. A vaccine is a biological preparation that improves immunity to a particular disease. A vaccine typically contains an agent that resembles a disease-causing microorganism (antigen), and is often made from weakened or killed forms of said microorganism, its toxins or one of its surface proteins. Although vaccines have been highly successful, new strategies need to be found to increase the effectiveness of some existing vaccines or to prevent or treat diseases such as malaria and HIV. Adjuvants can be used to modify or augment the effects of a vaccine by stimulating the immune system to respond to the vaccine more vigorously, and thus providing increased immunity to a particular disease. In particular, an adjuvant is a component that potentiates the immune responses to an antigen and/or modulates it towards the desired immune responses and nowadays includes soluble mediators and antigenic carriers that interact with surface molecules present on DC (e.g. LPS, Flt3L, heat shock protein), particulate antigens which are taken up by mechanisms available to APC but not other cell types (e.g. immunostimulatory complexes, latex, polystyrene particles) and viral/bacterial vectors that infect antigen presenting cells (e.g. vaccinia, lentivirus, adenovirus).

Live bacterial cells can be used as vehicles to deliver recombinant antigens. The evolution of genetic engineering techniques has enabled the construction of recombinant microorganisms capable of expressing heterologous proteins in different cellular compartments, improving their antigenic potential for the production of vaccines against viruses, bacteria, and parasites. For example, vaccines derived from an attenuated or avirulent version of a pathogen are highly effective in preventing or treating disease caused by that pathogen. In particular, it is known that such attenuated or avirulent pathogens can be altered to express heterologous antigens.

By using a carrier as source for a recombinant antigen, the presence of any additional products from the pathogen, which might be reactogenic, is ruled out (e.g. potential traces of co-purified products in acellular vaccines). The use of bacterial carriers is associated with several benefits such as low production batch preparation costs, increased shelf-life and stability compared to other formulations, easy administration and low delivery costs.

Non-limiting examples of bacterial species, which have been considered suitable as antigen delivery systems and exhibit a satisfactory immunogenicity profile are L. monocytogenes, Salmonella spp., V. cholera, Shigella spp., M. bovis BCG, Y. enterocolitica, B. anthracis, S. gordonii, Lactobacillus spp. and Staphylococcus spp.

A number of bacterial secretion systems, such as the Type I and type Ill secretion system, have been used to deliver the antigen of interest directly into the cytosol of antigen presenting cells (APCs), leading to the activation of effectors and memory T-CD8+ lymphocytes. Alternatively, the antigens can be expressed on the surface of the bacterial to induce immune responses. For this exposure, the antigen of interest is typically expressed fused to surface proteins of the vector (da Silva et al., Live bacterial vaccine vectors: an overview, Braz. J. Microbiol, 2014, 45(4)). Some examples of these fusion proteins include Lpp-OmpA, TolC, and FimH of E. coli and PulA of Klebsiella.

The LES according to present invention can be introduced into or attached to an antigen of interest, which will lead to the expression of said antigen on the surface of a bacterial cell and thereby enhances the antigenic properties. Accordingly, a peptide or polypeptide comprising the LES as described herein and preferably also the N-terminal signal peptide of a lipoprotein of Gram-negative bacteria comprising a lipobox motif located at the very end of the C-terminus of said signal peptide, wherein said lipobox motif consists of the amino acid sequence L(S/A)(A/G)C and is specifically recognizable by a signal peptidase type II as described herein, can be used for live or inactivated vaccine development to expose homologous or heterologous epitopes on human commensal or attenuated pathogenic bacterial cells to elicit antigen-specific antibody responses.

Moreover, the formation of fusion proteins of a protein of interest with transporter proteins, such as OmpA or TolC, or with proteins which are part of complex cell machineries, such as FimH, in order to achieve surface expression of the protein of interest, may not be without physiological consequences for the host bacteria. Proteins or polypeptides comprising solely a LES sequence, and preferably also the N-terminal signal peptide, according to the invention can be used to achieve an abundant coverage of the cell surface without affecting the bacterial physiology and is therefore advantageous over the existing methods for obtaining cell-surface expression of proteins.

Accordingly, another aspect of the invention is the use of the peptide or polypeptide, polypeptide precursor, nucleic acid, recombinant expression vector and recombinant host cell according to the invention for manufacturing a vaccine.

In particular embodiments, the peptide or polypeptide according to the invention is an antigen, or an epitope thereof.

The term “antigen” as used herein, refers to any polypeptide, or fragments thereof, capable of inducing an immune response on the part of the host organism and leads to the production of antibodies against it. Preferably the antigen has a size of 200 kDa or less, 150 kDa or less, 100 kDa or less, 50 kDa or less, more preferably, 100 kDa or less or 50 kDa or less. Preferably the antigen comprises at least 5, at least 6, at least 7, at least 8 amino acids, at least 9 amino acids or at least 10 amino acids, preferably at least 10 amino acids. Furthermore, the antigen is preferably surface exposed in its original host (the pathogen), in Bacteroidetes or in a non-pathogenic Bacteroidetes such as F. johnsoniae.

Addition of the LES and/or classical lipoprotein N-terminal signal peptide, preferably the N-terminal signal peptide of a lipoprotein of Gram-negative bacteria comprising a lipobox motif located at the very end of the C-terminus of said signal peptide, wherein said lipobox motif consists of the amino acid sequence L(S/A)(A/G)C (SEQ ID NO: 203) and is specifically recognizable by a signal peptidase type II as described herein, to the antigen will lead to the surface expression of said antigen. Accordingly, in particular embodiments, the polypeptide according to the invention is a homologous or heterologous antigen and is exposed to the surface of a host cell.

Host cell is preferably a cell which is able to express the antigen of interest. Furthermore, the host cell preferably comprises one or more transport systems and SPII peptidases which are able to recognize the classical lipoprotein signal peptide, preferably the N-terminal signal peptide of a lipoprotein of Gram-negative bacteria comprising a lipobox motif located at the very end of the C-terminus of said signal peptide, wherein said lipobox motif consists of the amino acid sequence L(S/A)(A/G)C (SEQ ID NO: 203) and is specifically recognizable by a signal peptidase type II as described herein, and/or LES consensus motif and can transport the antigen comprising said LES motif according to the invention to the cell surface. Preferably the host cell is a bacterial cell, more preferably a Gram-negative bacterial cell, even more preferably a bacterial cell from the Bacteroidetes phylum.

In particular embodiments, two or more different antigens of interest are expressed and exposed to the cell surface of the same host cell.

The host cells which express surface antigens according to the invention can be used to raise antibodies, such as polyclonal antibodies, in animals. This is achieved by injection of said host cells expressing surface antigens into laboratory or farm animals in order to raise high expression levels of antigen-specific antibodies in the serum, which can then be recovered from the animal. Polyclonal antibodies can be recovered directly from serum, while monoclonal antibodies are produced by fusing antibody-secreting spleen cells from immunized mice with immortal myeloma cell to create monoclonal hybridoma cell lines that express the specific antibody in cell culture supernatant.

Therefore, another aspect of the invention is the use of the polypeptide, polypeptide precursor, nucleic acid, recombinant expression vector and recombinant host cell according to the invention, for antibody production, preferably wherein said polypeptide is an antigen, more preferably a heterologous antigen.

In particular embodiments, two or more different polypeptides are expressed on the surface of the host cell. Preferably, said polypeptides are antigens, more preferably heterologous antigens.

In particular embodiments, the polypeptide according to the invention is exposed to the surface of a bacterial cell from the Bacteroidetes phylum, preferably Capnocytophaga canimorsus or Flavobacterium johnsoniae.

Recombinant proteins are used throughout biological and biomedical science. Recombinant DNA technology allows developing cells which produce large quantities of a desired protein. Recombinant expression allows the protein to be tagged (e.g. His-tag), which will facilitate purification, and to express the protein of interest with a higher fraction than is present in a natural source. Usually the protein purification protocol contains one or more precipitation and chromatographic steps and allows isolating the desired protein. If the protein of interest is not secreted by the organism into the surrounding solution, the first step of each purification process is the disruption of the cells containing the protein. This could be achieved by, for example by repeated freezing and thawing, sonication, high pressure homogenization or permeabilization by detergents and/or enzymes. Unfortunately, also proteases are released during cell lysis, which will start digesting the proteins in the solution. Hence, the extract should be handled fast and cooled to slow down the reaction. Alternatively, one or more protease inhibitors can be added to the lysis buffer immediately before cell disruption. Sometimes it is also necessary to add DNAse in order to reduce the viscosity of the cell lysate caused by a high DNA content.

The polypeptide comprising a LES according to present invention and preferably also the N-terminal signal peptide of a lipoprotein of Gram-negative bacteria comprising a lipobox motif located at the very end of the C-terminus of said signal peptide, wherein said lipobox motif consists of the amino acid sequence L(S/A)(A/G)C and is specifically recognizable by a signal peptidase type II as described herein, can be used as a new system to allow producing immediately pure proteins by-passing the fastidious purification steps of cytosolic or secreted recombinant proteins. This can be achieved by cloning in the 5′ region of the gene of interest an oligonucleotide that would generate a lipoprotein with (i) a classical lipoprotein signal peptide comprising a lipobox motif which is specifically recognized by a signal peptidase type II, preferably the N-terminal signal peptide of a lipoprotein of Gram-negative bacteria comprising a lipobox motif located at the very end of the C-terminus of said signal peptide, wherein said lipobox motif consists of the amino acid sequence L(S/A)(A/G)C (SEQ ID NO: 203) and is specifically recognizable by a signal peptidase type II as described herein, (ii) the LES according to present invention and (iii) a cleavage site of a specific protease (e.g. TEV). Next, the gene of interest is expressed in a bacterium of the Bacteroidetes group (e.g. C. canimorsus or preferably a biosafety class I organism like Flavobacterium johnsoniae). After culture, a bacteria covered with the protein of interest is obtained. The protein of interest remains attached to the OM by the lipid anchor. Subsequently, the bacteria can be washed and resuspended in a protein-free buffer. Then, use of specific proteases cleaving the introduced cleavage site will release the recombinant protein. After pelleting of the bacteria, a solution containing only the protein of interest and the protease is obtained. The protease can be easily removed by use of, for example, immuno-beads. Accordingly, pure recombinant protein can be obtained by a minimal number of purification steps using the polypeptide, nucleic acid, recombinant expression vector and recombinant host cell according to present invention.

Therefore, another aspect of the invention is the use of the polypeptide, polypeptide precursor, nucleic acid, recombinant expression vector and recombinant host cell according to the invention for protein production and purification.

Bacterial surface display is a protein engineering technique that allows linking the function of a protein with the gene that encodes it, finding target proteins with desired properties (e.g. enzyme substrates, cell-specific peptides or protein-binding peptides) and making cell-specific affinity ligands. Libraries of polypeptides can be displayed on the surface of bacteria and can subsequently be screened using fluorescence-activated cell sorting, magnetic activated cell sorting and/or iterative selection procedures.

Accordingly, another aspect of the invention is the use of the polypeptide, polypeptide precursor, nucleic acid, recombinant expression vector and recombinant host cell according to the invention for performing bacterial display.

In particular embodiments, two or more different polypeptides are expressed on the surface of the host cell.

Bacteria which expose enzymes to their cell surface can be immobilized and used as an alternative for enzyme immobilization to a solid support or matrix. Bacteria can be immobilized by, for example, carrier binding, self-aggregation or entrapment. Enzymes exposed on the surface of bacteria are especially useful when the enzymes of interest are difficult or expensive to extract or when a series of enzymes are required in the reaction. Bacteria exposing enzymes to their cell surface can act as whole-cell biocatalysts. Reactions catalyzed by immobilized whole-cell biocatalysts can be reactions involving single enzymes, multiple enzyme systems, optionally with cofactors or a complete metabolic pathway. Typically, the bacteria exposing the enzymes are put into contact with a medium containing substrate or effector or inhibitor molecules, allowing the enzymatic reaction to take place. Immobilized enzymes can be used for numerous applications, including industrial production of antibiotics, beverages or amino acids, as drug delivery systems, in the diagnosis and treatment of diseases, in the production of food (e.g. syrups from fruits and vegetables), in the production of bio-diesel, in the waste water treatment of sewage and industrial effluents, in textile industry (e.g. scouring, bio-polishing), for dirt removal of clothes, etc. For example, a bacteria expressing amino-acylase on their cell surface can be used for the production of L-amino acids.

Accordingly, another aspect of the invention is the use of the polypeptide, polypeptide precursor, nucleic acid, recombinant expression vector and recombinant host cell according to the invention for whole-cell based biocatalytic applications, preferably wherein said polypeptide is an enzyme or catalytically active fragment thereof.

In particular embodiments, two or more different polypeptides are expressed on the surface of the host cell. Preferably, said polypeptides are enzymes, or catalytically active fragment thereof.

Biosensors combine a bio-recognition component (‘bioreceptor’) with a physicochemical detector and are, inter alia, useful for bioprocess monitoring, determination of drug residues in food, drug discovery, glucose monitoring in diabetes patients or environmental applications. The bio-recognition component can be a host cell, such as bacteria, expressing bioreceptors of interest on their cell surface. Interaction of the bioreceptor with an analyte of interest in a sample can be measured by the physicochemical detector which outputs a measurable signal proportional to the presence of the target analyte in the sample. The bioreceptor/analyte interactions can be based on antibody/antigen, enzymes, nucleic acids/DNA, cellular structures/cells or biomimetic materials interactions.

Accordingly, another aspect of the invention is the use of the polypeptide, polypeptide precursor, nucleic acid, recombinant expression vector and recombinant host cell according to the invention for manufacturing biosensors.

Host cells, such as bacteria, which express polypeptides capable of binding contaminants onto their cell surface can be used for a process called bio-adsorption (‘biosorption’), wherein contaminants are adsorbed onto the cellular surface of the host cell. The host cell's biosorption capacities can be enhanced by modifying the set of polypeptides which are expressed on the cell surface of said host cell. For example, bacteria expressing polypeptides which specifically recognize and bind chemicals or heavy metals of interest can be used for the removal of said specific harmful chemicals or heavy metals of interest from the environment. At an industrial scale, biosorption is often performed using sorption columns to which an effluent containing contaminants is fed.

Accordingly, another aspect of the invention is the use of the polypeptide, polypeptide precursor, nucleic acid, recombinant expression vector and recombinant host cell according to the invention for biosorption applications.

The present invention further also relates to the use of the polypeptide, the polypeptide precursor, the nucleic acid or the expression vector according to the invention, wherein said polypeptide and/or said polypeptide precursor comprises and/or wherein said nucleic acid or said expression vector encodes an antigen, or epitope thereof, or an enzyme, or catalytically active fragment thereof, which will be exposed to the surface of a host cell comprising said polypeptide, said polypeptide precursor, said nucleic acid and/or said expression vector.

In particular embodiments, said host cell is a Bacteroidetes, preferably C. canimorsus or Flavobacterium johnsoniae.

The present invention is further illustrated in the following non-limiting examples.

### EXAMPLES

Materials and Methods

1. Bacterial Strains and Growth Conditions

Bacterial strains used in this study are listed in Table S1. Escherichia coli strains were routinely grown in lysogeny broth (LB) at 37° C. C. canimorsus strains were routinely grown on heart infusion agar (Difco) supplemented with 5% sheep blood (Oxoid) plates (SB plates) for 2 days at 37° C. in the presence of 5% CO₂. To select for plasmids, antibiotics were added at the following concentrations: 100 μg/ml ampicillin (Amp), 50 μg/ml kanamycin (Km) for E. coli and 10 μg/ml erythromycin (Em), 10 μg/ml cefoxitin (Cfx), 20 μg/ml gentamicin (Gm) for C. canimorsus.

2. Heat-Inactivation of Normal Human Serum (NHS)

Ten ml aliquots of NHS (S1-Liter; Millipore) were thawed and heat-inactivated at 56° C. for 1 h. The Heat-Inactivated Human Serum (HIHS) was then dispensed into single use aliquots and stored at −20° C.

3. Construction of siaC and mucG Expression Plasmids

Plasmids and primers used in this study are listed in Table S2 and S3 respectively. siaC (Ccan_04790) was amplified from 100 ng C. canimorsus 5 genomic DNA with primers 4159 and 7696 using Q5 High-Fidelity DNA Polymerase (M0491S; New England Biolabs). The initial denaturation was at 98° C. for 2 min, followed by 30 cycles of amplification (98° C. for 30 s, 52° C. for 30 s, and 72° C. for 2 min) and finally 10 min at 72° C. After purification, the fragment was digested using NcoI and XhoI restriction enzymes and cloned into plasmid pMM47.A, leading to plasmid pFL117. mucG (Ccan_17430) was cloned in the same way except that primers 7182 and 7625 were used for amplification and that the fragment was cloned into pPM5.

Site-specific point mutations were introduced by amplifying separately the N- and C-terminal part of each gene using forward and reverse primers harboring the desired mutations in their sequence in combination with primers 4159 and 7696 for siaC and 7182 and 7625 for mucG. Both PCR fragments were purified and then mixed in equal amounts for PCR using the PrimeStar HS DNA Polymerase (R010A; Takara). The initial denaturation was at 98° C. for 2 min, followed by 30 cycles of amplification (98° C. for 10 s, 60° C. for 5 s, and 72° C. for 3 min 30 s) and finally 10 min at 72° C. Final PCR products were then cleaned, digested using NcoI and XhoI restriction enzymes and cloned into plasmids pMM47 or pPM5 for siaC and mucG respectively. The incorporation of the desired point mutations in all inserts was confirmed by sequencing. Plasmids expressing siaC and mucG variants were transferred to C. canimorsus 5 siaC and mucG deletion strains respectively by electroporation.

4. SDS PAGE and Western Blotting

Bacteria grown for 2 days on SB plates were collected, washed once with PBS, and resuspended in one ml PBS at an OD₆₀₀ of 1, corresponding to approximately 5×10⁸ bacteria. Bacteria were collected by centrifugation for 3 min at 5,000 g and resuspended in 100 μl SDS PAGE buffer (1% SDS, 10% glycerol, 50 mM dithiothreitol, 0.02% bromophenol blue, 45 mM Tris, pH 6.8). Samples were heated for 5 min at 96° C. and 5 μl were loaded on 12% SDS PAGE gels. After gel electrophoresis, proteins were transferred onto nitrocellulose membrane (1060008; GE Healthcare) and analyzed by Western blot using rabbit anti-SiaC or anti-MucG antisera as primary antibodies and swine-HRP anti-rabbit (P0217; Dako) as secondary antibody. Proteins were detected using LumiGLO (54-61-00; KPL) according to manufacturer's instructions.

5. Human Salivary Mucin Degradation

Fresh human saliva was collected from healthy volunteers and filter-sterilized using 0.22 μm filters (Millipore). Bacteria grown for 2 days on SB plates were collected, washed once with PBS, and set to an OD₆₀₀ of 1. One hundred μl of bacterial suspension (approximately 5×10⁷ bacteria) were then mixed with 100 μl of human saliva and incubated for 240 min at 37° C. As negative control, 100 μl of saliva was incubated with 100 μl PBS. Samples were then centrifuged for 5 min at 13,000 g, the supernatant carefully collected and loaded on 10% SDS PAGE gels. Mucin degradation was monitored by lectin staining with PNA agglutinin (DIG glycan differentiation kit, 11210238001; Roche) according to manufacturer's instructions. Mucin degradation was estimated by loss or reduction of PNA staining as compared to the negative control.

6. Outer Membrane Protein Purification

Outer membrane proteins were isolated as described in (Wilson et al., Analysis of the outer membrane proteome and secretome of Bacteroides fragilis reveals a multiplicity of secretion mechanisms. PloS one, 2015 10(2):e0117732 and Kotarski et al., Isolation and characterization of outer membranes of Bacteroides thetaiotaomicron grown on different carbohydrates. J Bacteriol, 1984. 158(1): p. 102-9) with several modifications. All steps were carried out on ice unless otherwise stated. All sucrose concentrations are expressed as percentages of w/v in 10 mM HEPES (pH 7.4). Bacteria collected from 2 plates were washed 2 times with 30 ml 10 mM HEPES (pH 7.4) before being resuspended in 4.5 ml of 10% sucrose. Bacterial cells were then disrupted by 2 passages through a French press at 35,000 psi. The lysate was collected and centrifuged for 10 min at 16,500 g to pellet insoluble material. The crude cell extract was then layered on top of a sucrose step gradient composed of 1.33 ml of 70% sucrose and 6 ml of 37% sucrose and centrifuged at 100,000 g (28,000 rpm) for 70 min at 4° C. in a SW41 Ti rotor. The yellow material above the 37% sucrose solution and at the 10%/37% interface, corresponding to soluble and enriched inner membrane proteins, was collected and diluted to 7 ml with 10 mM HEPES (pH 7.4). The high density band at the 37%/70% interface, corresponding to enriched outer membrane proteins, was collected and diluted to 7 ml with 10 mM HEPES (pH 7.4). Membranes from both fractions were then centrifuged at 320,000 g (68,000 rpm) for 90 min at 4° C. in a 70.1 Ti rotor. The supernatant of the yellow material fraction, corresponding to soluble proteins, was transferred to a fresh tube and stored at −20° C. The pellet of the same tube, corresponding to a mixture of inner and outer membrane fractions, was resuspended in 1 ml of 40% sucrose and stored at −20° C. The supernatant of the outer membrane protein band was discarded, the pellet resuspended in 7 ml of 10 mM HEPES (pH 7.4) containing 1% Sarkozyl (L5777; Sigma-Aldrich) and incubated at room temperature for 30 min with constant agitation. The outer membrane was then centrifuged at 320,000 g for 60 min at 4° C. in a 70.1 Ti rotor, resuspended in 7 ml of 100 mM Na₂CO₃ (pH 11) and incubated at 4 interface, corresponding to enriched outer membrane proteins, was collected and diluted to 7 ml with 10 mM HEPES (pH 7.4). Membranes from both fracy, the purified outer membrane was resuspended in 200 to 400 μl unbuffered 40 mM Tris and stored at −20° C. Protein concentration of all fractions was assessed using the Bio-Rad Protein Assay (500-0006; Bio-Rad) according to manufacturer's instructions. One to 2 μg of total protein of total cell lysate and outer membrane fraction were loaded on 12% SDS PAGE gels. After gel electrophoresis, proteins were transferred onto nitrocellulose membrane and analyzed by Western blot.

7. Immunofluorescent Labelling for Flow Cytometry and Microscopy Analysis

Bacteria grown for 2 days on SB plates were collected, washed once with PBS, and resuspended in one ml PBS to an OD₆₀₀ of 0.1. 5 μl of bacterial suspensions (approximately 3×10⁵ bacteria) were used to inoculate 2.5 ml of DMEM (41965-039; Gibco) containing 10% heat-inactivated human serum (HIHS) in 12-well plates (665 180; Greiner Bio-one). Bacteria were harvested after 23 h of growth at 37° C. in the presence of 5% CO₂, washed twice with PBS, and resuspended in 1 ml PBS. The optical density at 600 nm was measured and equivalent amounts corresponding to approximately 3×10⁷ bacteria were collected for each strain. Bacteria were resuspended in 200 μl PBS containing 1% BSA (w/v) and incubated for 30 min at room temperature. Bacteria were then centrifuged, resuspended in 200 μl of a primary antibody dilution (1:1500 rabbit anti-SiaC antiserum or 1:500 rabbit anti-MucG antiserum) and incubated for 30 min at room temperature. Following centrifugation, bacterial cells were washed 3 times before being resuspended in 200 μl of a secondary antibody 1:500 dilution (donkey anti-rabbit coupled to Alexa Fluor 488; A-21206; Invitrogen) and incubated for 30 min at room temperature in the dark. Following centrifugation, bacterial cells were washed 3 times before being resuspended in 200 μl of 4% PFA (w/v) and incubated for 15 min at room temperature in the dark. Finally, bacteria were centrifuged, washed once and resuspended in 700 μl of PBS. For flow cytometry analysis, samples were directly analyzed with a BD FACSVerse™ (BD Biosciences) and data were processed with BD FACSuite™ (BD Biosciences). For microscopy analysis, labeled bacteria were added on top of poly-L-lysine-coated coverslips and were allowed to adhere for 30 min at room temperature. After removal of bacterial suspension, coverslips were washed 3 times, mounted upside down on glass slides and allowed to dry overnight at room temperature in the dark. All microscopy images were captured with an Axioscop (Zeiss) microscope with an Orca-Flash 4.0 camera (Hamamatsu) and Zen 2012 software (Zeiss). Images were processed using ImageJ software. As control, samples were prepared in parallel as described above except that rabbit pre-immunization serum was used for labeling.

8. In Vivo Radiolabeling with [³H] Palmitate, Immunoprecipitation and Fluorography

Bacteria were grown overnight as described above for immunofluorescent labelling, except that bacteria were grown in 5 ml medium in 6-well plates (657 160; Greiner Bio-one). After 18 h of incubation, [9,10-³H] palmitic acid (32 Ci/mmol; NET043; Perkin-Elmer Life Sciences) was added to a final concentration of 50 μCi/ml and incubation was continued for 6 h. Bacteria were then collected by centrifugation, washed 2 times with 1 ml PBS and pellets were stored at −20° C. until further use. Pellets were resuspended in 300 μl PBS containing 1% Triton™ X-100 (28817.295; VWR) and vortexed 10 sec to lyse bacteria. Lysates were centrifuged 2 min at 14,000 g and the supernatant was transferred into a new tube. MucG proteins were immuno-precipitated by addition of 15 μl MucG antiserum for 90 min at room temperature with constant agitation. In parallel, 20 μl of Protein A agarose slurry (P3476; Sigma-Aldrich) were washed 2 times with 500 μl wash buffer (0.1% Triton™ X-100 in PBS), saturated with 500 μl 0.2% BSA (w/v) for 30 min and washed again 2 times with wash buffer. The Protein A agarose slurry was then added to the cell lysate and incubation was continued for 30 min at room temperature with constant agitation. Samples were then centrifuged at 14,000 g for 2 min and the supernatant was discarded. Pellets were washed 5 times with 500 μl wash buffer. Bound proteins were eluted by addition of 50 μl SDS PAGE buffer and heating for 10 min at 95° C. Samples were centrifuged again and supernatants were carefully separated from the agarose beads and loaded on 10% SDS PAGE gels. After gel electrophoresis, gels were fixed in a 25/65/10 isopropanol/water/acetic acid solution overnight and subsequently soaked for 30 min in Amplify (NAMP100; Amersham) solution. Gels were vacuum dried and exposed to SuperRX autoradiography film (Fuji) for 13-21 days until desired signal strength was reached.

Lipoproteins Multiple Sequence Alignment

The sequences of 40 lipoproteins previously identified as being part of the surface proteome of C. canimorsus 5 (Manfredi, P., et al., The genome and surface proteome of Capnocytophaga canimorsus reveal a key role of glycan foraging systems in host glycoproteins deglycosylation. Mol Microbiol, 2011. 81(4): p. 1050-60) were retrieved from the Uniprot database (Release 2015_12; UniProt: a hub for protein information. Nucleic Acids Res, 2015. 43(Database issue): p. D204-12). Additionally, 2 C. canimorsus 5 proteins (F9YSD4 and F9YTT3) detected at the bacterial surface but predicted to harbour an SPI signal were reanalysed with the PATRIC database (Wattam, A. R., et al., PATRIC, the bacterial bioinformatics database and analysis resource. Nucleic Acids Res, 2014. 42(Database issue): p. D581-91) and found to possess an SPII signal and thus considered lipoproteins, rendering a final list of 43 surface exposed predicted lipoproteins (Table S4). The SPII cleavage site of each protein was then predicted using the LipoP software (1.0 Server, default settings), showing that all proteins possess one clear SPII cleavage site. Accordingly, protein sequences were trimmed to their predicted mature form. Lists corresponding to either full-length protein sequences or 15 amino acids downstream of the +1 cysteine were generated. Datasets were then submitted to multiple sequence alignment using the MAFFT online tool (version 7.268, default settings) and the output was analysed using the Jalview software (version 2.9.0b2). The final consensus sequence logo was drawn using WebLogo (version 2.8.2, default settings). The sequences of the 17 C. canimorsus outer membrane lipoproteins presumably facing the periplasm (Manfredi, P., et al., The genome and surface proteome of Capnocytophaga canimorsus reveal a key role of glycan foraging systems in host glycoproteins deglycosylation. Mol Microbiol, 2011. 81(4): p. 1050-60) were processed in the same way (Table S5). The sequences of the 22 previously identified proteinase K sensitive Bacteroides fragilis NCTC 9343 surface exposed lipoproteins (Wilson M M, Anderson D E, & Bernstein H D (2015) Analysis of the outer membrane proteome and secretome of Bacteroides fragilis reveals a multiplicity of secretion mechanisms. PloS one 10(2):e0117732) were processed in the same way (Table S6). Forty-two Flavobacterium johnsoniae UW101 predicted SusD-like lipoproteins were identified in the PULDB of the CAZY database (Terrapon N, Lombard V, Gilbert H J, & Henrissat B (2015) Automatic prediction of polysaccharide utilization loci in Bacteroidetes species. Bioinformatics 31(5):647-655.), the corresponding sequences extracted from the Uniprot database and processed as described above (Table S7).

9. Statistical Analysis

All data are presented as mean±standard deviation (SD). Statistical analyses were done by one-way ANOVA followed by Bonferroni test using the GraphPad Prism version 5.00 for Windows, GraphPad Software, La Jolla Calif. USA, www.graphpad.com. A P value 0.05 was considered statistically significant.

### Experiment 1: In Silico Identification of a Putative Lipoprotein Export Signal

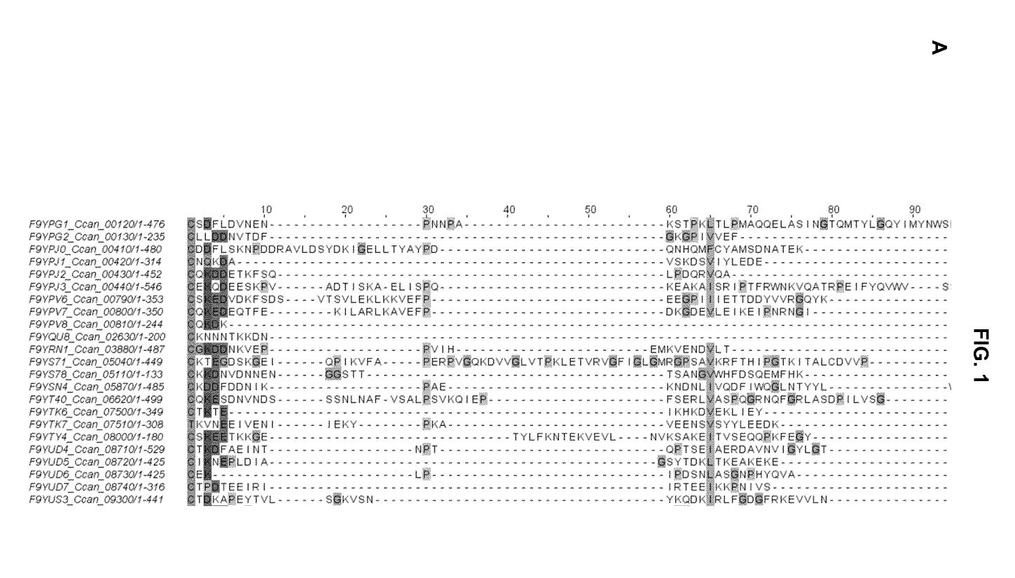

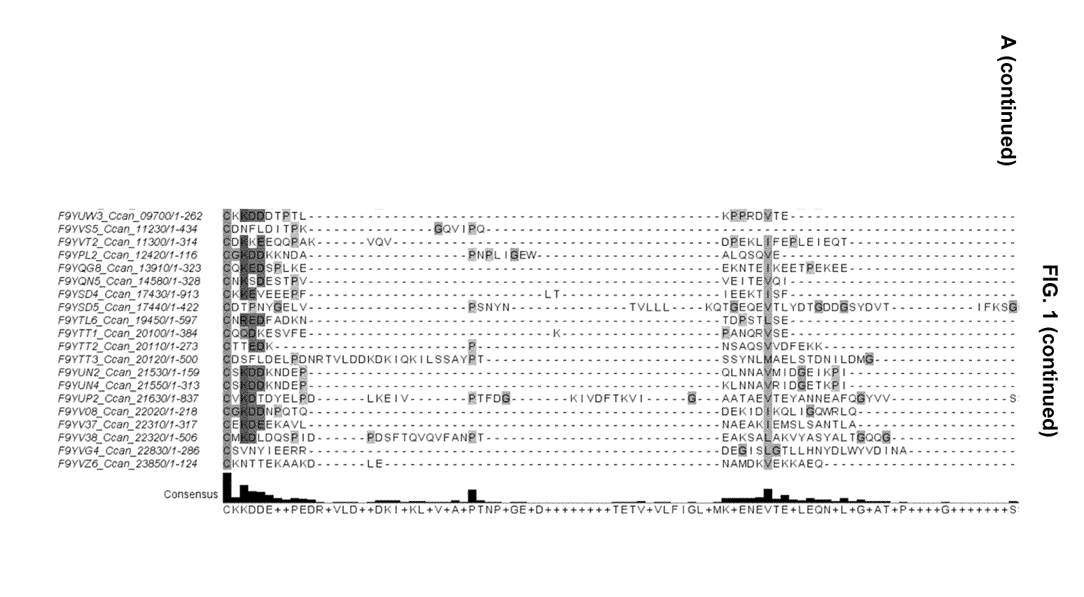

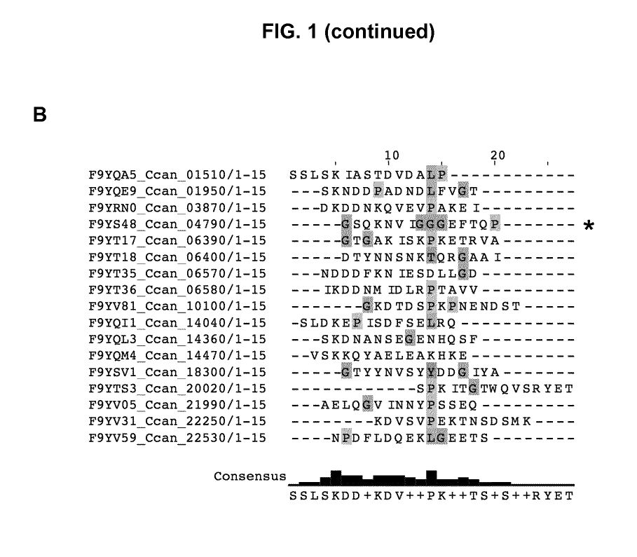

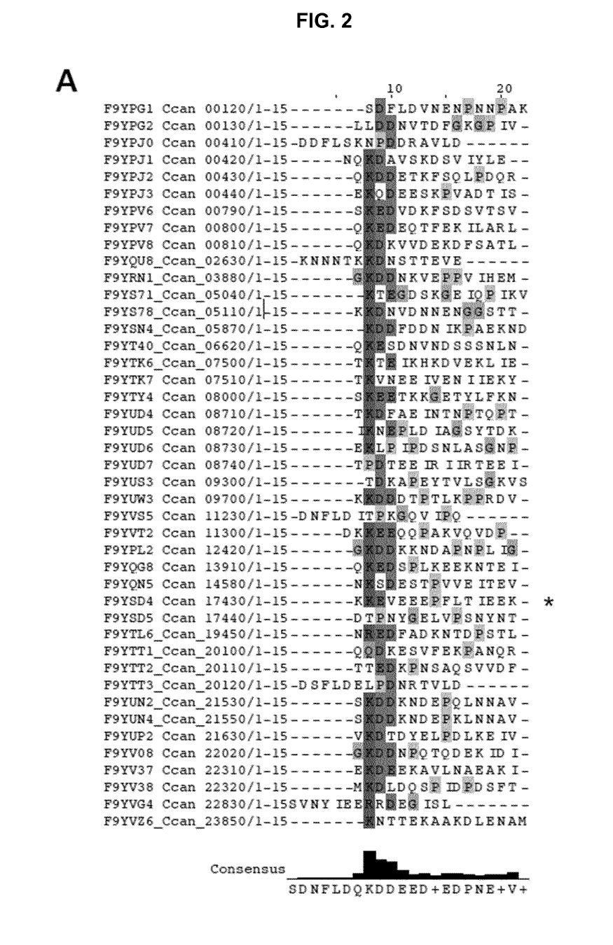

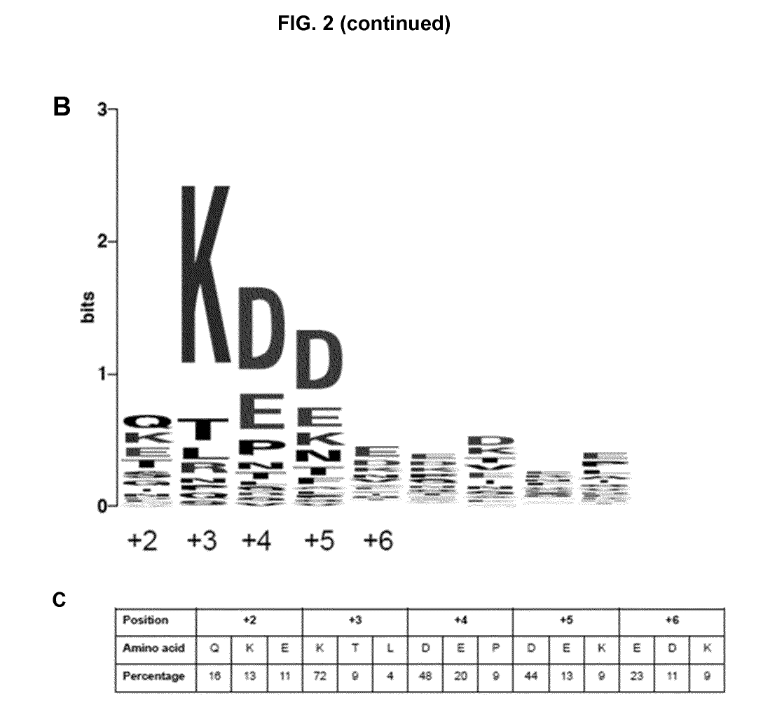

In order to see if a specific amino acid motif would be responsible for the targeting of lipoproteins to the bacterial surface, the Inventors examined in detail the sequences of the 43 lipoproteins detected at the surface of C. canimorsus 5 (Manfredi, P., et al., The genome and surface proteome of Capnocytophaga canimorsus reveal a key role of glycan foraging systems in host glycoproteins deglycosylation. Mol Microbiol, 2011. 81(4): p. 1050-60). The Inventors first identified the SPII cleavage site using the LipoP software and then aligned the mature lipoproteins using MAFFT. Several residues seemed to be conserved throughout the protein sequences but did not appear to constitute a clear motif (data not shown). However, a lysine (K) residue followed by either an aspartate (D) or a glutamate (E) residue appeared to be conserved in close proximity to the N-terminal cysteine at position +1 (FIG. 1A). This was refined by a second alignment using only the 15 N-terminal residues of the mature lipoprotein but excluding the +1 cysteine to avoid that this invariant residue would influence the analysis (FIG. 2A). The consensus motif identified corresponded to Q-K-D-D-E (SEQ ID NO: 16) (FIG. 2B) with a conservation of 16, 72, 48, 44 and 23% respectively (FIG. 2C). It consists thus of a positively charged residue (K) at position +3 followed by two to three negatively charged amino acids (D and/or E) at positions +4, +5 and +6 immediately after the lipidated cysteine. In order to see whether this motif is specific to the surface-exposed lipoproteins, the same analysis was performed on OM lipoproteins which were not detected at the bacterial surface and were thus supposed to face the periplasm. Among these lipoproteins, only a conserved D or E residue at position +3 was identified (FIG. 1B), suggesting that the QKDDE (SEQ ID NO: 16) peptide could indeed be a bona fide lipoprotein export signal (LES).

### Experiment 2: The QKDDE Sequence Leads to Surface Localization of the Periplasmic Lipoprotein SiaC

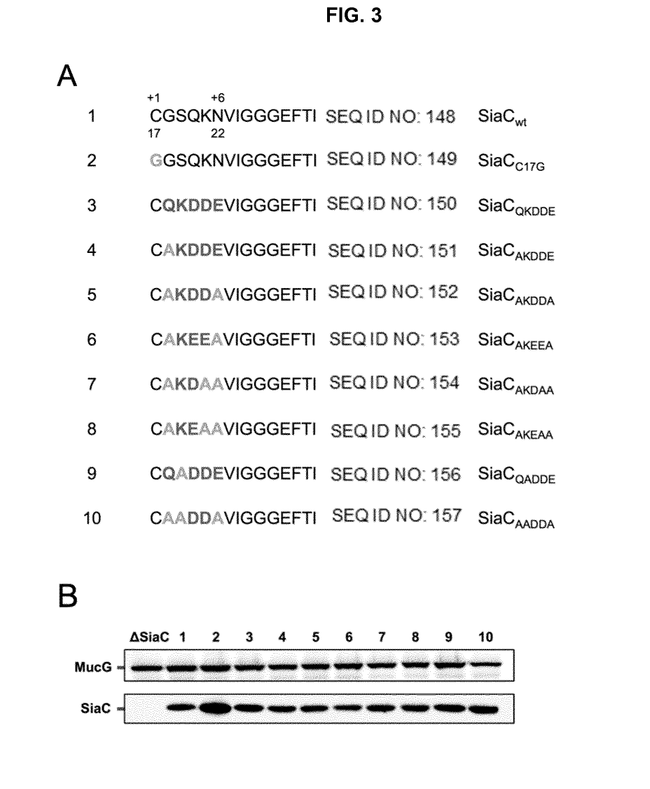

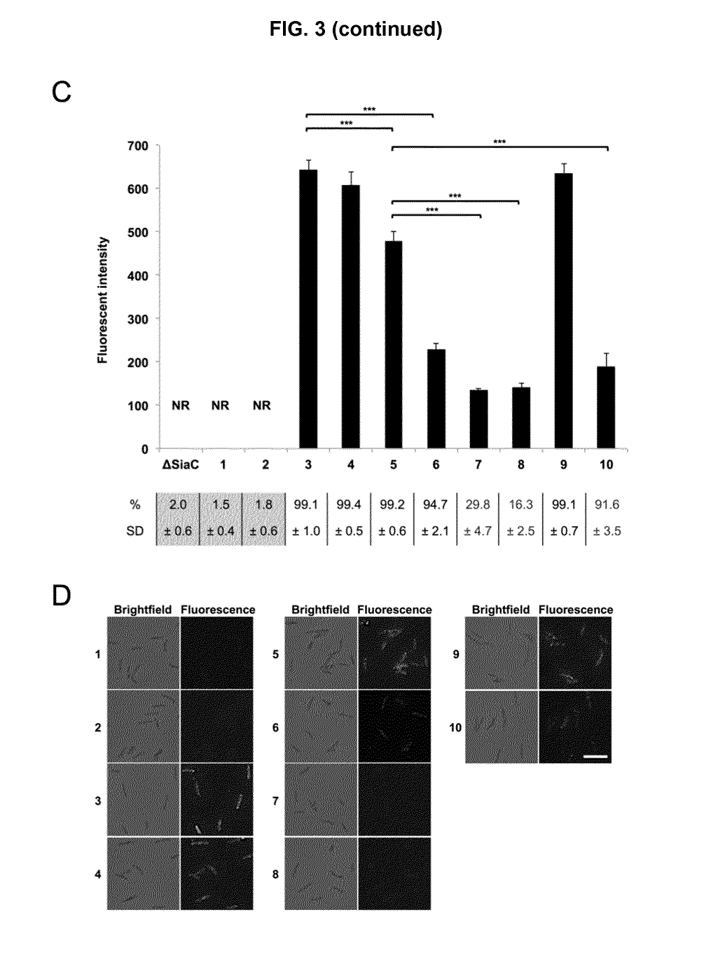

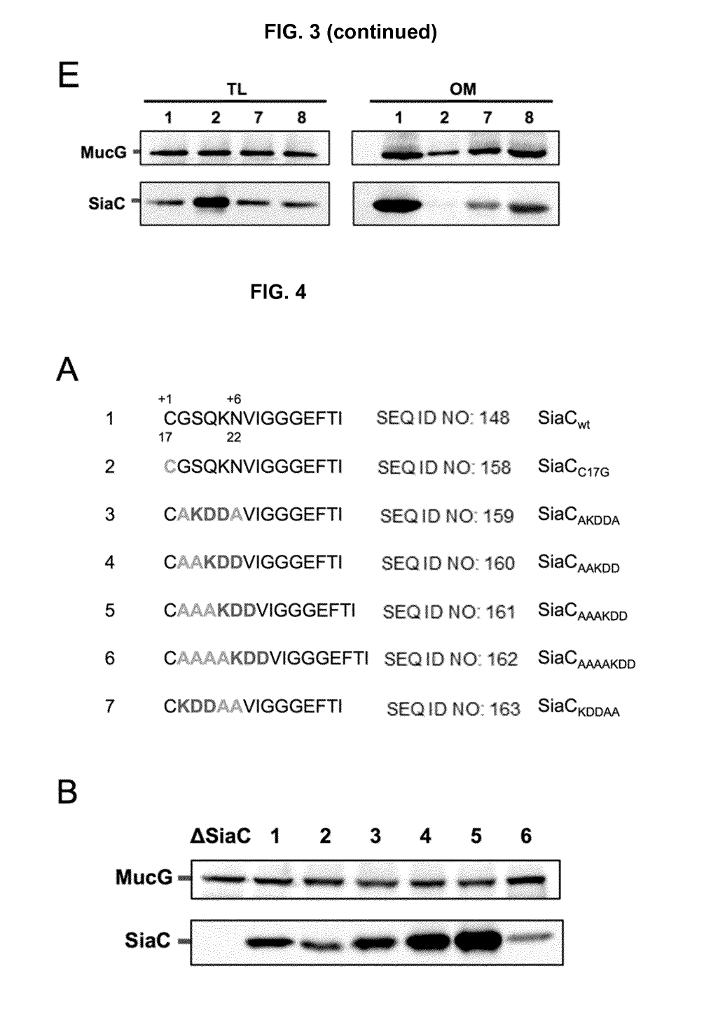

To verify this hypothesis, the Inventors introduced the QKDDE (SEQ ID NO: 16) motif in the sequence of the C. canimorsus sialidase (SiaC) protein, an outer membrane lipoprotein previously shown to face the periplasm (Mally, M., et al., Capnocytophaga canimorsus: a human pathogen feeding at the surface of epithelial cells and phagocytes. PLoS Pathog, 2008. 4(9): p. e1000164 and Renzi, F., et al., The N-glycan glycoprotein deglycosylation complex (Gpd) from Capnocytophaga canimorsus deglycosylates human IgG. PLoS Pathog, 2011. 7(6): p. e1002118). To do so, the Inventors cloned in a C. canimorsus expression vector genes encoding either the wt SiaC, SiaC_{C17G} that would not be acylated or SiaC_{+2QKDDE+6} carrying the hypothetical export signal instead of the wt residues 18 to 22 and the Inventors expressed these genes in a siaC deletion strain (FIG. 3A). The Inventors first verified that the expression of the three proteins was similar (FIG. 3B) and then monitored the surface exposure by immuno-fluorescence, using flow cytometry and fluorescence microscopy on intact cells (FIGS. 3C and D). Interestingly, while wt SiaC and SiaC_{C17G} were, as expected, both undetectable at the bacterial surface by either methods, expression of the SiaC_{+2QKDDE+6} protein led to a strong fluorescence as determined by flow cytometry and microscopy (FIGS. 2C and D) indicating that the protein was surface exposed. These results indicated that the identified consensus sequence is sufficient on its own to drive transport of a lipoprotein to the surface.

### Experiment 3: Determination of the Minimal Consensus Allowing Surface Localization of SiaC

The Inventors then asked whether all the 5 residues of the QKDDE (SEQ ID NO: 16) consensus are required to form a functional LES. The Inventors first substituted the least conserved amino acids, namely Q18 and E22, by alanines, generating constructs SiaC_{+2AKDDE+6} and SiaC_{+2AKDDA+6} (FIG. 3A). After monitoring protein expression (FIG. 3B), flow cytometry and microscopy showed that both constructs localized to the surface (FIGS. 3C and D), although to a slightly lower extent than SiaC_{+2QKDDE+6}. This indicated that the KDD motif is sufficient to target lipoproteins to the surface and that the residues at position +2 and +6 downstream of the +1 cysteine are thus dispensable. The Inventors then tested if glutamate was able to functionally replace aspartate (SiaC_{+2AKEEA+6}) (FIG. 3A) since both residues were enriched in the consensus (FIG. 2C). As shown in FIGS. 3C and D, substitution of the aspartates with two glutamates did not prevent the surface localization but led to a clear reduction of fluorescence in line with the lower conservation of glutamate at position +4 and +5 (FIG. 2C), indicating that in C. canimorsus surface lipoproteins aspartate could be preferred over glutamate. Noteworthy, only the total amount of SiaC displayed at the bacterial surface was affected by these mutations, as all analyzed mutant cells were labeled by the SiaC antiserum (FIG. 3C), suggesting that these mutations only decreased the efficiency of transport of SiaC to the surface.

The Inventors then generated two SiaC constructs harboring only either KD or KE (SiaC_{+2AKDAA+6} and SiaC_{+2AKEAA+6}) (FIG. 3A) but these two residues alone turned out to be very poor LES since only 29.8±4.7 (SiaC_{+2AKDAA+6}) and 16.3±2.5% (SiaC_{+2AKDAA+6}) of the cells displayed the protein at their surface (FIG. 3C). In addition, the fluorescent intensity was weak: 28.2 and 29.4% respectively of the intensity observed for the SiaC_{+2QKDDE+6} reference (FIG. 3C). In order to verify that these constructs were not impaired in their transport to the OM, the Inventors confirmed the localization of the proteins by western blot on the isolated outer membrane fraction (FIG. 3E). This supported their hypothesis that K(D/E)₂ represents the minimal LES. These constructs also suggested that a functional LES might require an overall negative charge, indicated by the fact that KDD is allowing efficient transport of SiaC to the surface while KD is not (FIG. 3C).

Finally, the Inventors investigated the importance of the highly conserved lysine residue at position +3 (FIG. 3A). Unexpectedly, substitution of K alone (SiaC_{+2QADDE+6}) had only a slight impact on the display of SiaC at the bacterial surface (FIGS. 3C and D). However, removal of both K and Q (SiaC_{+2AADDA+6}) led to a more than 60% decrease of fluorescent intensity as compared to SiaC_{+2AKDDA+6}. Since the glutamine residue itself was not critical (SiaC_{+2AKDDE+6}, FIG. 3C), one has to conclude that either the +2 Q or the +3 K are required for an efficient LES.

Taken together, these data indicate that the minimal export motif allowing surface localization of SiaC is composed of only two negatively charged amino acids (aspartate and/or glutamate) preceded by a positively charged or polar residue. Based on the consensus, the Inventors thus defined the minimal LES as being K(D/E)₂, taking into account the low conservation of Q at position +2.

### Experiment 4: Positional Effect of the Minimal LES on SiaC Surface Localization

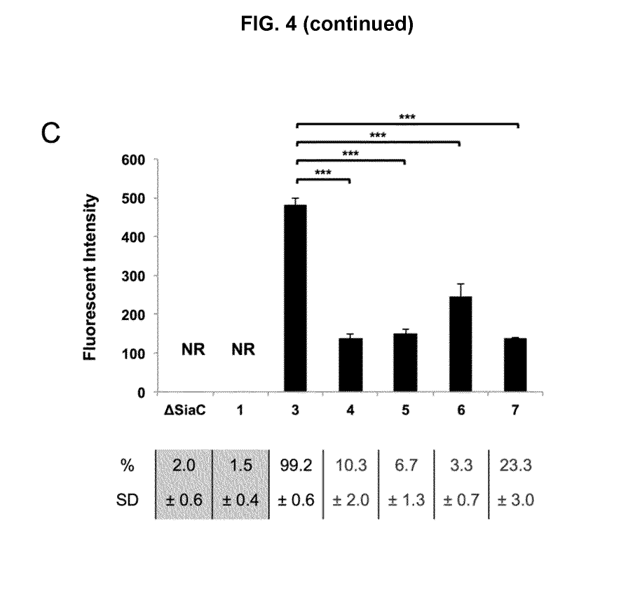

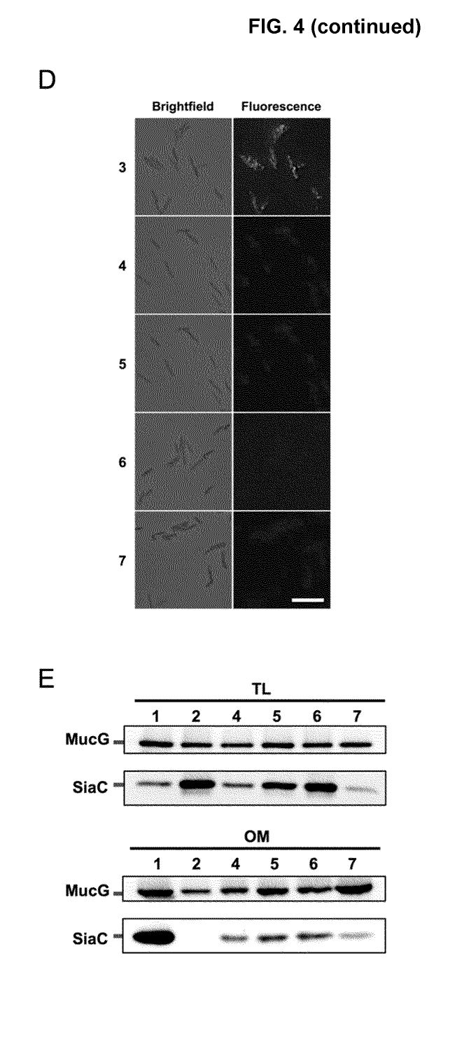

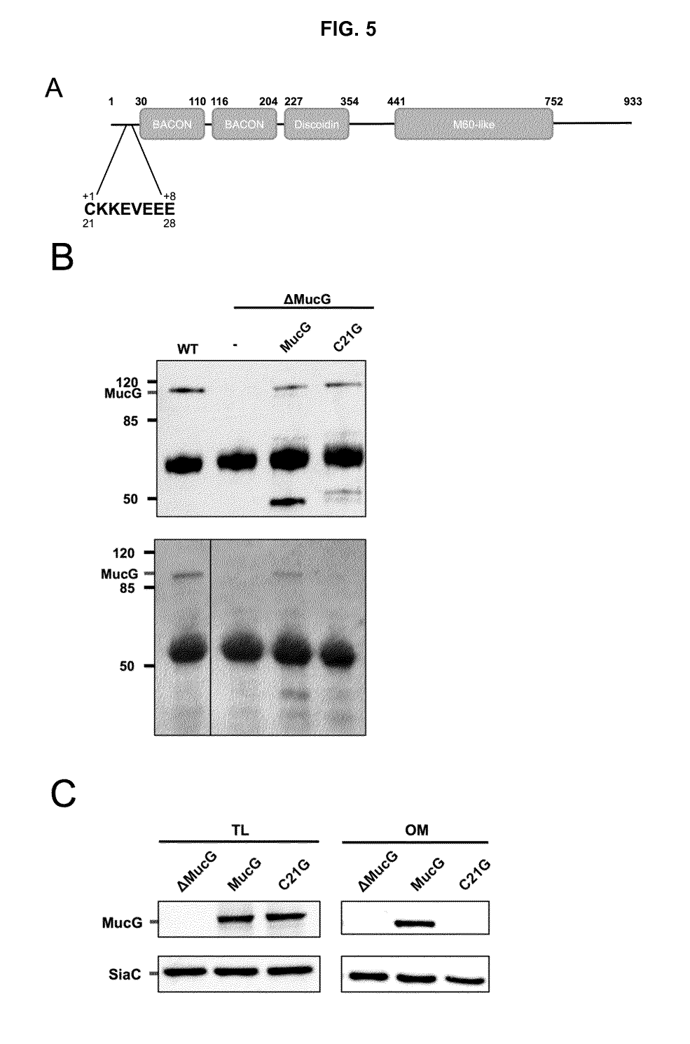

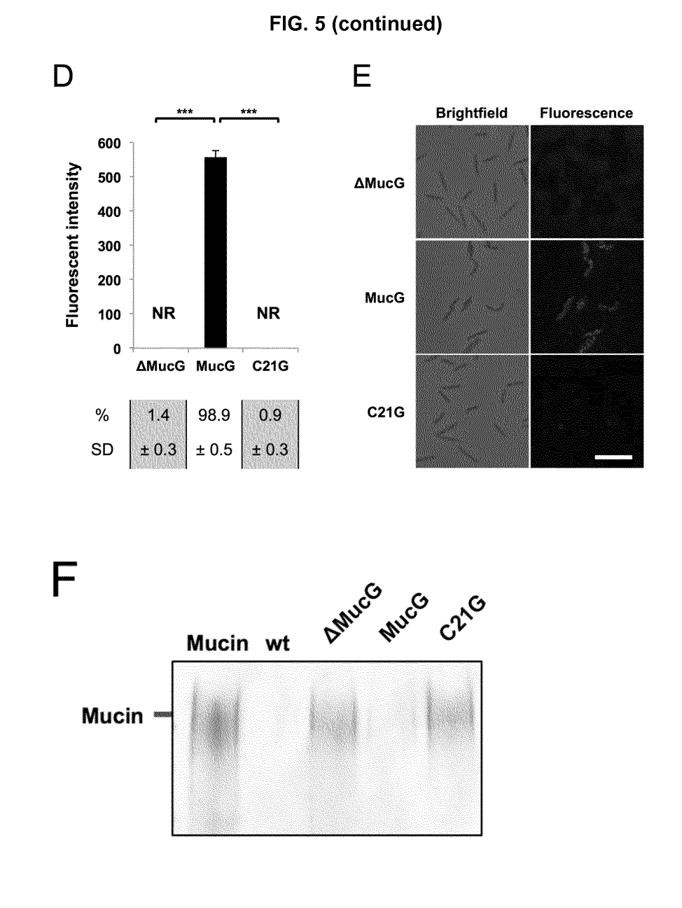

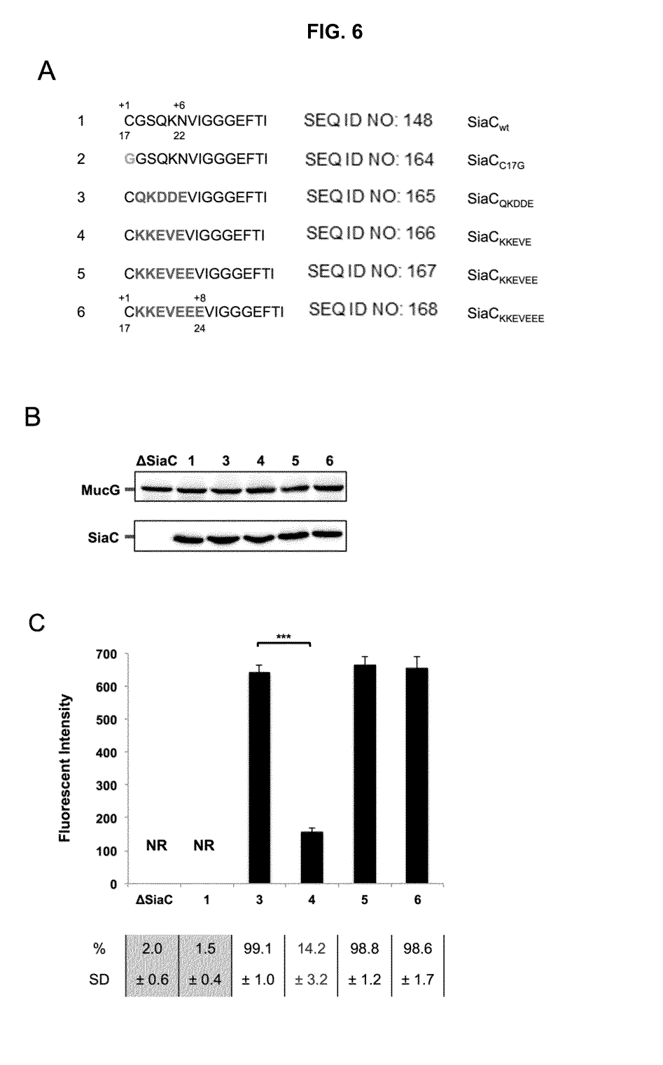

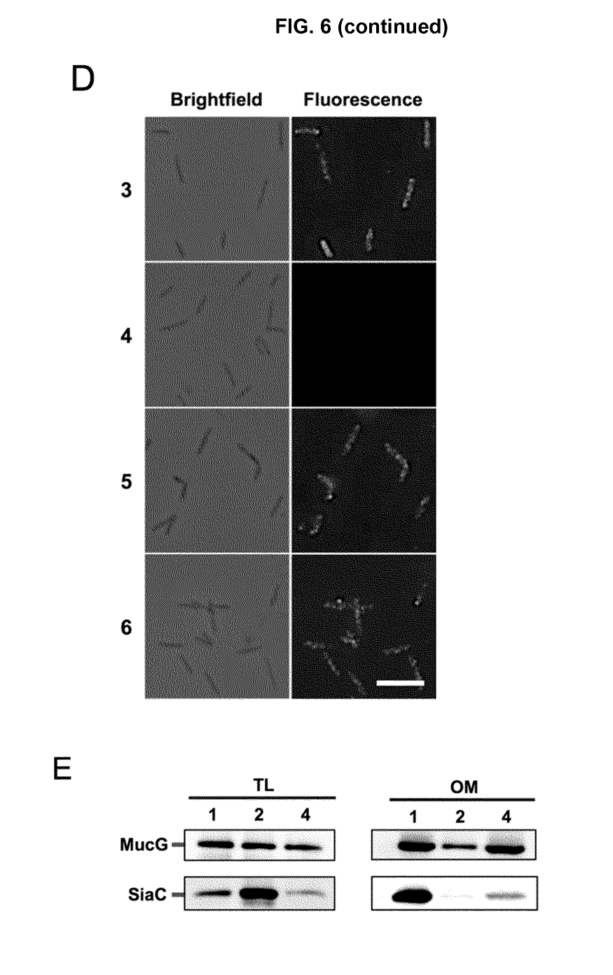

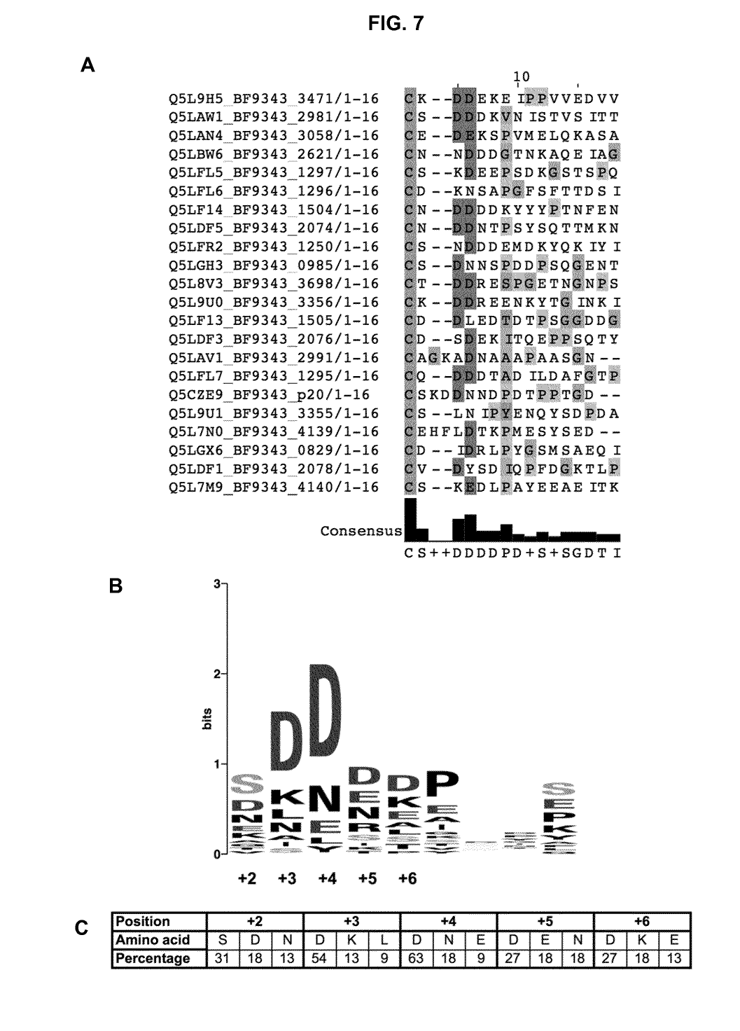

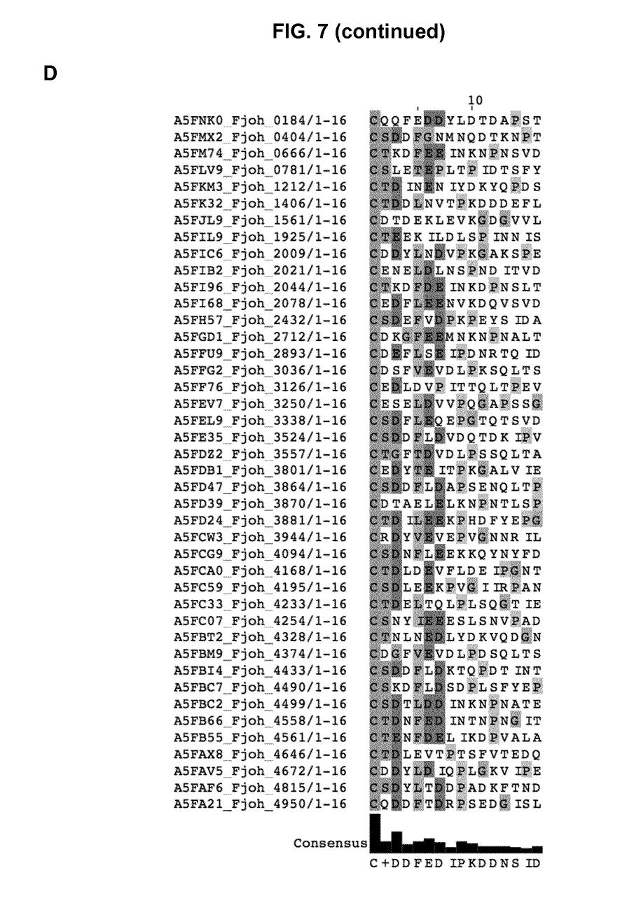

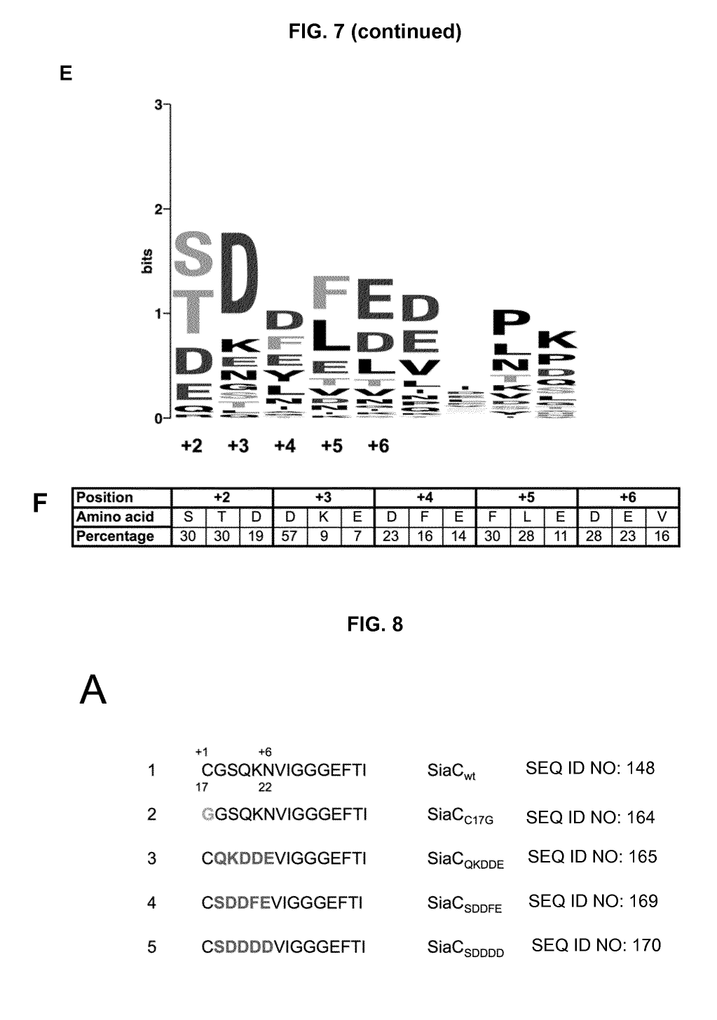

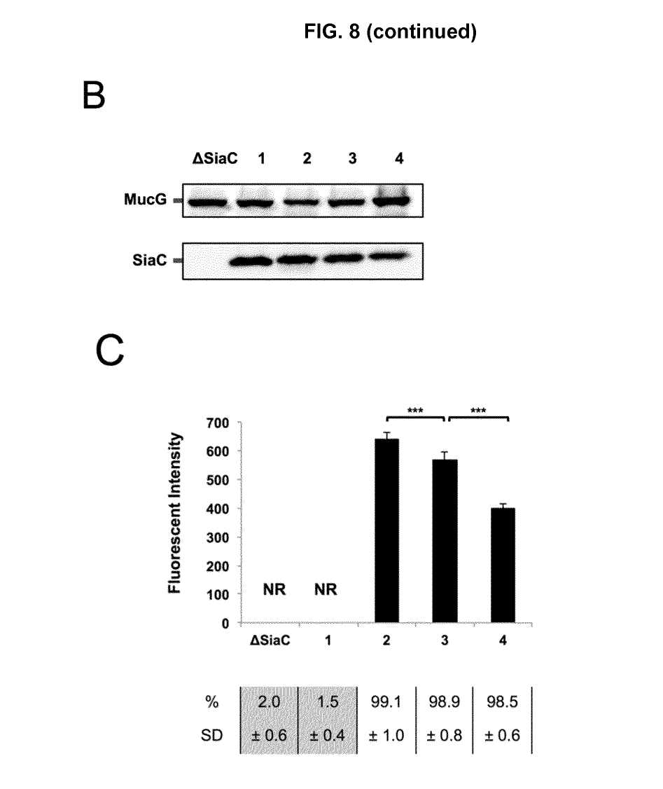

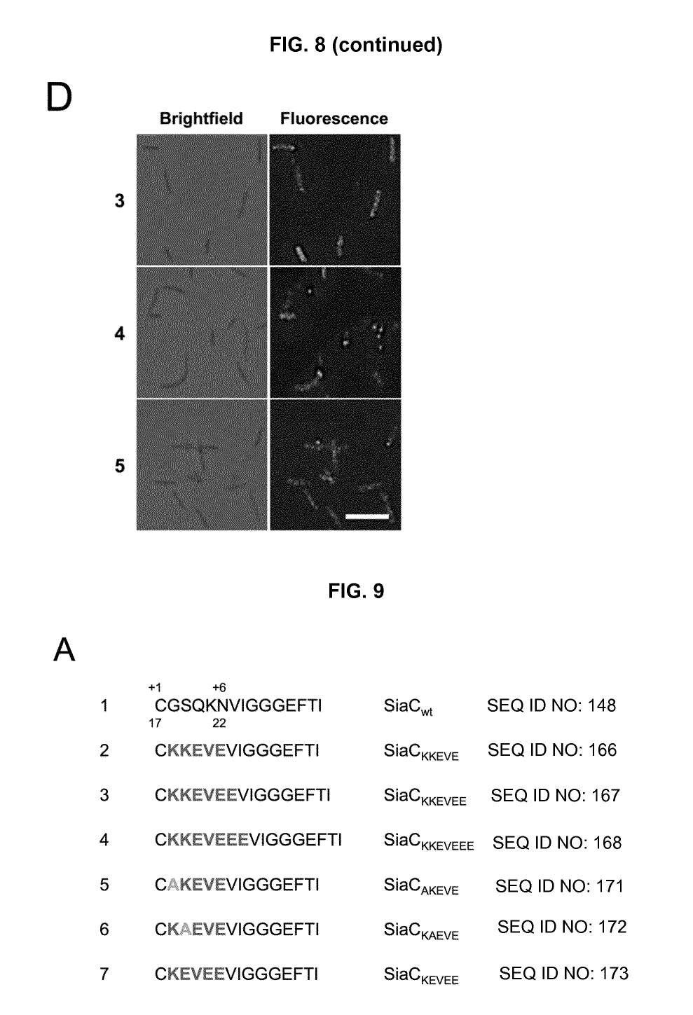

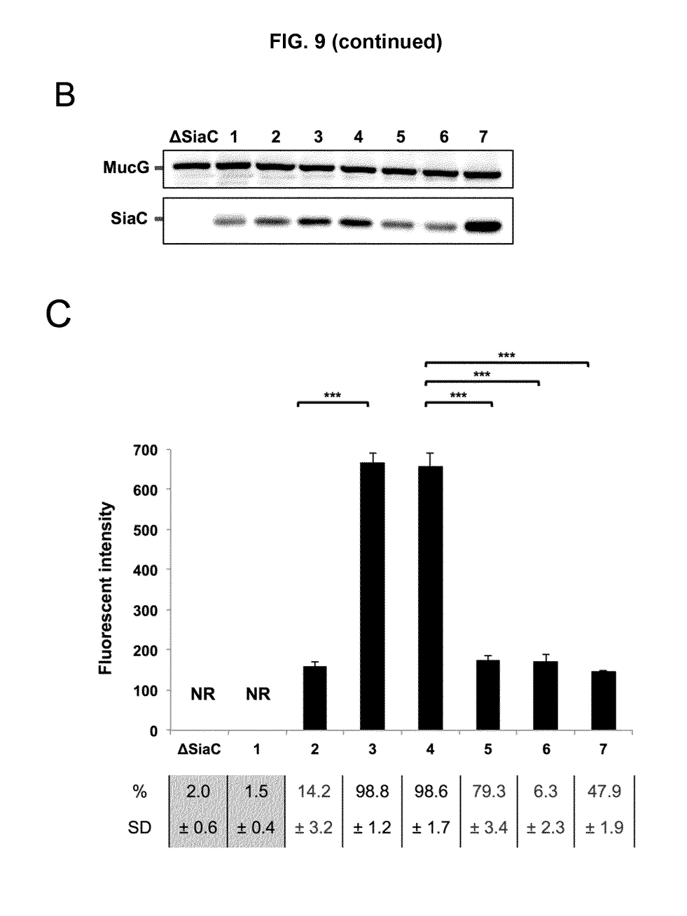

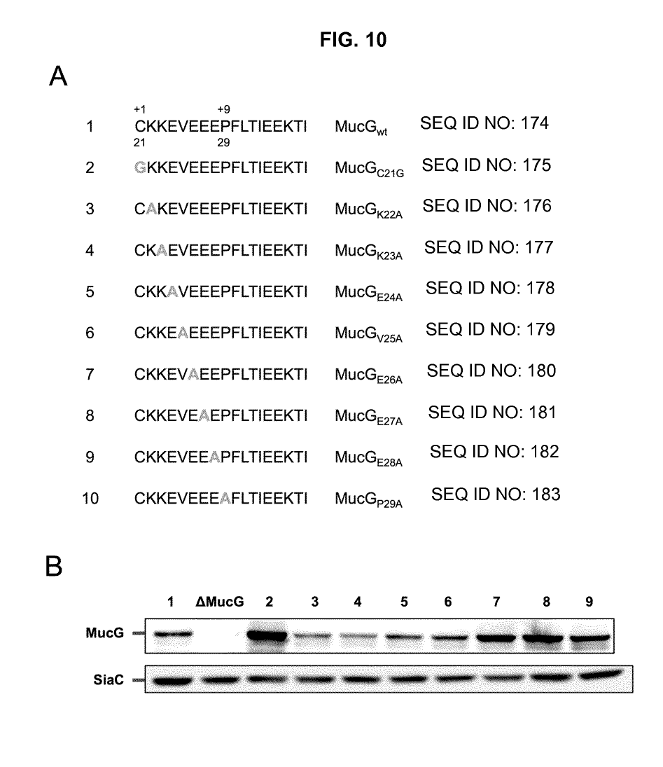

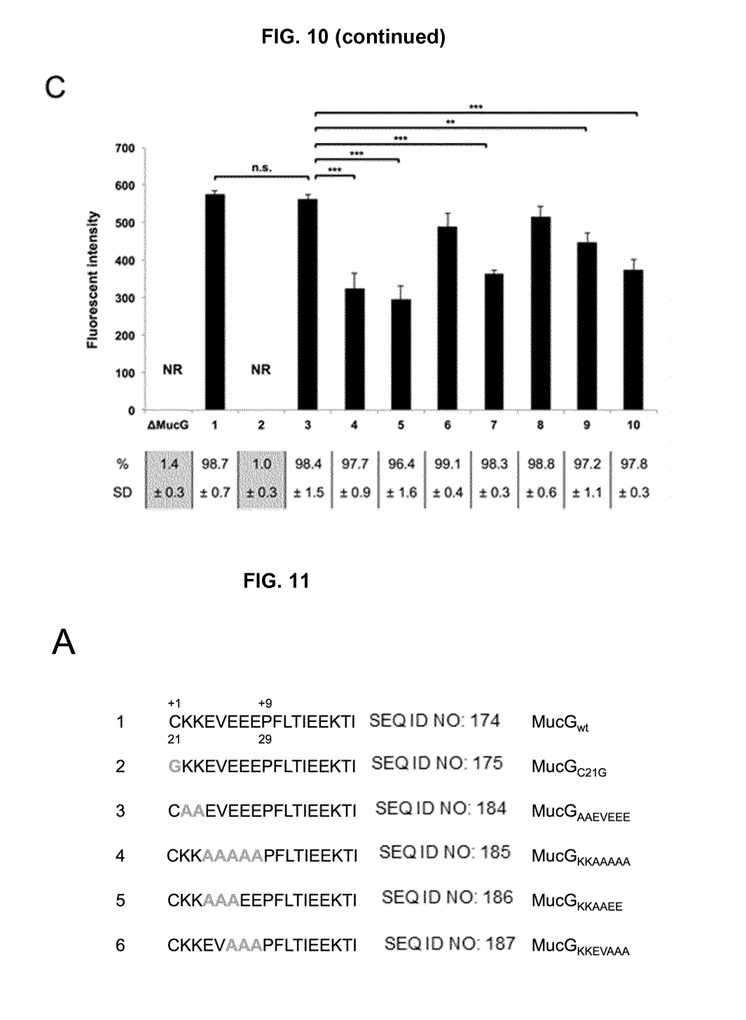

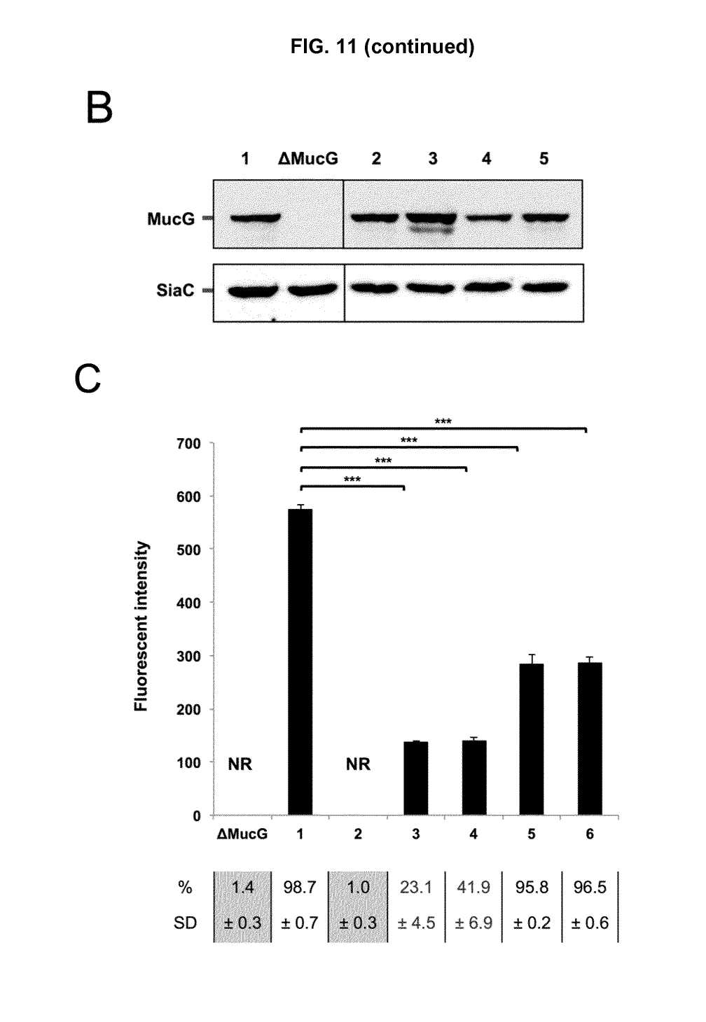

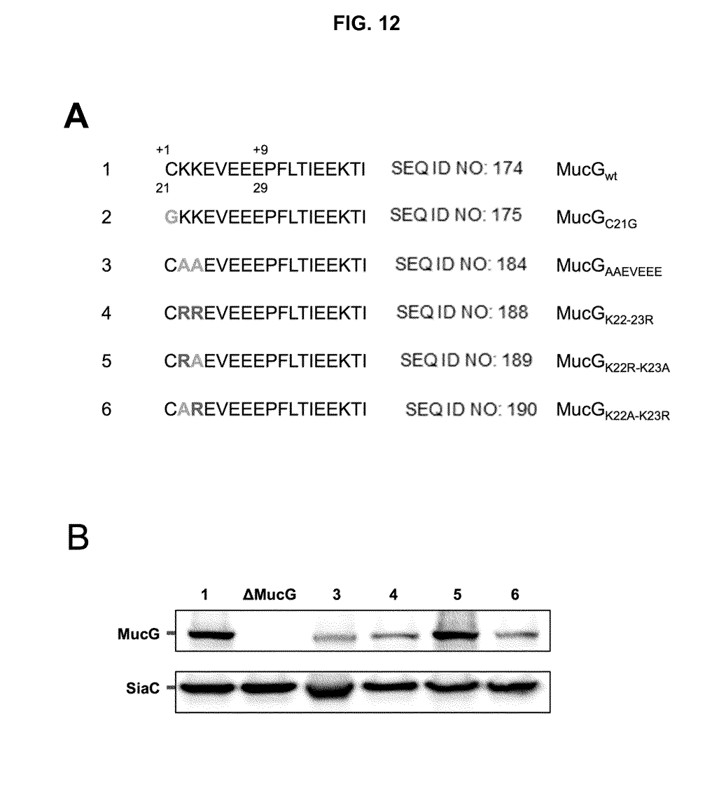

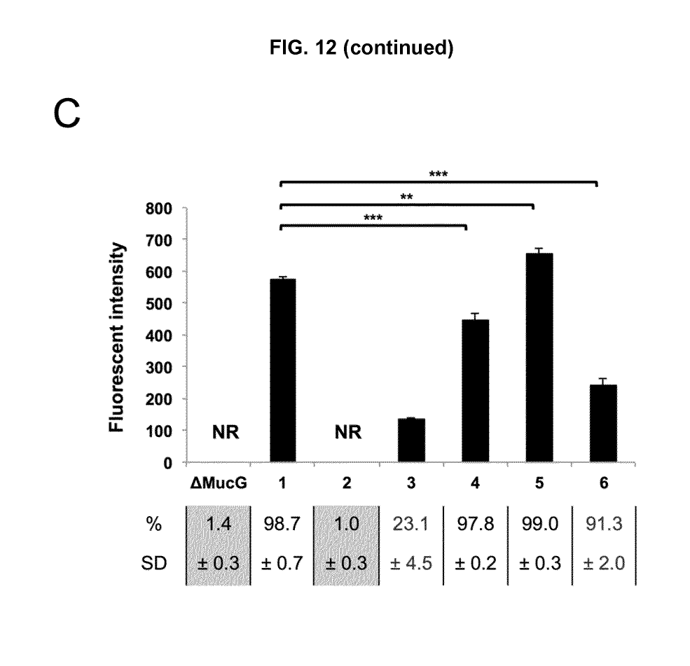

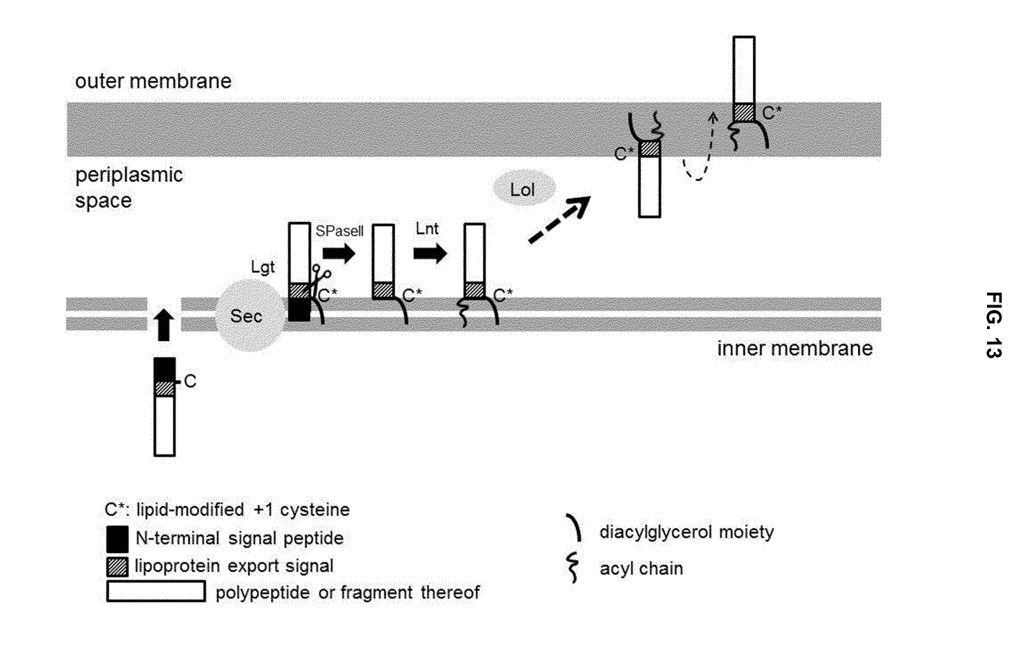

The initial alignment showed that K had a strong conservation at position +3 (72%), a low conservation at position +2 (13%) (FIG. 2C) and was completely absent from position +4. In contrast, D and E were conserved at positions +4, +5 and +6 (48, 44 and 11% for D and 20, 13 and 23% for E respectively) (FIG. 2C) and completely absent from position +3. This suggested that not only the composition of the export motif was crucial, but also its position relative to the +1 cysteine. The Inventors therefore generated constructs in which the KDD motif was separated from the +1 cysteine by zero, two, three or four alanine residues (FIG. 4A). Although the four proteins were expressed (FIG. 4B), none was exported as efficiently as the one where only one alanine separated the KDD motif from the +1 cysteine (FIGS. 4C and D). Interestingly, all the proteins were anchored to the OM, thus again indicating that only the last step of transport to the surface was affected by these mutations. (FIG. 4E), Overall, these data highlight the importance of the position of the LES relative to the +1 cysteine.

### Experiment 5: The MucG Export Signal Determines Surface Exposure SiaC

In order to confirm the robustness of their results, the Inventors analyzed the export motif of a naturally surface exposed lipoprotein of C. canimorsus. To this aim the Inventors chose the previously characterized PUL9 encoded MucG protein (Renzi, F., et al., Glycan-foraging systems reveal the adaptation of Capnocytophaga canimorsus to the dog mouth. MBio, 2015. 6(2): p. e02507). The Inventors first checked by palmitate labeling and cell fractionation that MucG is indeed an OM lipoprotein and the Inventors confirmed its surface localization by immunofluorescence and enzymatic assay (FIG. 5A-F). According to FIG. 2, the Inventors assumed that the LES of MucG is either KKEVEEE (SEQ ID NO: 49) or part of this sequence (FIG. 5A), located directly C-terminally of the +1 cysteine. Interestingly, the hypothetical MucG LES differs slightly from the consensus sequence, due to the presence of two lysine residues and the presence of a non-polar valine in between the glutamate residues. The Inventors therefore replaced residues 18 to 22 of SiaC by residues 22 to 26 (SiaC_{+2KKEVE+6}), 22 to 27 (SiaC_{+2KKEVEE+7}) or 22 to 28 (SiaC_{+2KKEVEEE+8}) from the hypothetical MucG LES (FIG. 6A) and confirmed expression of the proteins (FIG. 6B). Interestingly, only the SiaC_{+2KKEVEE+7} and SiaC_{+2KKEVEEE+8} proteins localized to the bacterial surface, as shown by flow cytometry and microscopy (FIGS. 6C and D). In contrast, SiaC_{+2KKEVE+6} was surface exposed in only 14.2±3.2% of the cells (FIGS. 6C and D) but nevertheless anchored to the OM (FIG. 6E). Since the latter construct is as close from the consensus LES (X-K-(D/E)₂-X) (SEQ ID NO: 40 to 47), wherein said LES is located directly C-terminally of the +1 cysteine, as the two other ones, another feature must play a role. This feature is likely to be the presence of two positively charged residues in combination with only two negatively charged residues, making the overall signal region neutral rather than negatively charged. This fact also agrees with their previous results showing that SiaC was not transported to the cell surface when the LES was not negatively charged (FIGS. 3C and D).

Taken together, the data with the MucG export signal add two new informations: first, the canonical LES (X-K-(D/E)₂-X) (SEQ ID NO: 191 to 194), wherein said LES is located directly C-terminally of the +1 cysteine, may be interrupted by a small hydrophobic residue and, second, the overall charge of the LES must be negative. This reinforces the conclusion that KDD is sufficient to promote surface localization of SiaC, provided the +2 and +6 residues do not interfere with the global negative charge of the consensus motif.

### Experiment 6: The LES is Conserved in the Bacteroidetes Phylum

The Inventors next wanted to see if the identified LES would be present in surface lipoproteins of other Bacteroidetes species. The Inventors therefore took advantage of the recently published B. fragilis surfome analysis (Wilson, M. M., D. E. Anderson, and H. D. Bernstein, Analysis of the outer membrane proteome and secretome of Bacteroides fragilis reveals a multiplicity of secretion mechanisms. PLoS One, 2015. 10(2): p. e0117732) and performed a bioinformatic analysis on the N-terminus of the lipoproteins that were identified at the surface (FIG. 7A-C). The N-term turned out to be also enriched in negatively charged amino acids in close proximity to the +1 cysteine (SDDDD, SEQ ID NO: 1) (FIG. 8A). However, unlike in the C. canimorsus LES, the aspartate residues were majorly located at position +3 and +4 instead of +4 and +5. Additionally, this region was not enriched in positively charged amino acids but in a polar residue. This is not in strong contradiction with the C. canimorsus LES since the Inventors have shown that, in C. canimorsus, the lysine residue may be substituted by an alanine provided the glutamine was present at position +2 (FIGS. 3C and D). Thus, the Inventors hypothesize that SDDDD (SEQ ID NO: 1) forms the LES of B. fragilis. Since C. canimorsus and B. fragilis are phylogenetically distant, the Inventors wanted to see if the LES would be more similar in a closer related species, namely Flavobacterium johnsoniae. Since no surfome analysis has been performed on this bacterium, the Inventors recovered the sequences of all predicted SusD-homologs, supposedly surface exposed lipoproteins, from the PULDB of the CAZY database. The Inventors next analyzed the N-termini of these lipoproteins and derived the consensus sequence SDDFE (SEQ ID NO: 2) (FIG. 7D-F). Interestingly, this sequence appears closer to the LES of B. fragilis than to the LES of C. canimorsus in the sense that the N-terminus of these lipoproteins is enriched in a polar residue rather than in a positively charged residue. However, negatively charged amino acids are still predominant in this region of the proteins.

### Experiment 7: The LES from B. fragilis and F. johnsoniae is Functional in C. canimorsus

Finally the Inventors tested if the canonical sequences predicted for B. fragilis (SDDDD, SEQ ID NO: 1) and F. johnsoniae (SDDFE) (SEQ ID NO: 2) would represent a functional LES in C. canimorsus (FIG. 8A). Both sequences were inserted in SiaC and the recombinant proteins were tested in C. canimorsus 5. As shown in FIGS. 8C and D, both constructs turned out to be surface localized.

Taken together, these data show that the LES identified in C. canimorsus is quite conserved in other Bacteroidetes genera and that the LES from Bacteroides and Flavobacteria allow surface transport of lipoproteins in Capnocytophaga. Interestingly, not all features of the C. canimorsus LES, such as the conservation of the +3 K or the position of the negatively charged amino acids, are conserved in other Bacteroidetes. However, the three identified LES shared the requirement for a positively charged or polar residue followed by 2 or 3 negatively charged residues, giving an overall negative charge in close proximity to the +1 cysteine. This is thus confirming the evidence of a shared novel pathway for lipoprotein export in this phylum of Gram-negative bacteria.

### Experiment 8: Additional Investigation of the MucG LES in SiaC

The Inventors deduced from their in silico analysis that the MucG LES corresponded to 22-KKEVEEE-28 (SEQ ID NO: 49)(FIG. 5A), which they then confirmed when introducing this sequence into SiaC (FIG. 6). Interestingly, insertion of only 22-KKEVE-26 (SEQ ID NO:64) into SiaC led to very poor surface localization of the protein (FIGS. 6C and D), which confirmed their previous finding of the requirement of a negatively charged LES. Indeed, the 22-KKEVE-26 (SEQ ID NO:64) peptide is neutral in charge due to the presence of two positive and two negative charges, while 22-KKEVEE-27 (SEQ ID NO:63) and 22-KKEVEEE-28 (SEQ ID NO:49), both leading to clear surface localization of SiaC (FIG. 6), are negatively charged thanks to additional glutamate residues.

In order to further confirm this hypothesis, the Inventors constructed two versions of the SiaC_{KKEVE} protein in which we mutated one of the lysine residues into alanine (SiaC_{+2KAEVE+6} and SiaC_{+2AKEVE+6} respectively) thus rendering the signal's overall charge negative (FIG. 9A). Following western blot analysis to confirm expression (FIG. 9B), the Inventors monitored the presence of these SiaC variants at the cell surface by flow cytometry (FIG. 9C). Interestingly, the SiaC_{+2AKEVE+8} variant was surface localized in 79.3±3.4% of the cells (FIG. 9C), although the total amount of SiaC displayed by each cell was lower than in the SiaC_{+2KKEVEE+7} and SiaC_{+2KKEVEEE+8} constructs (approximately 25%). This represents a dramatic increase as compared to SiaC_{+2KKEVE+6} and confirmed that removal of one positively charged amino acid does indeed favor surface targeting. The fact that only a small amount of SiaC was transported to the surface in this context could reflect their previous finding that glutamate is less efficient at promoting SiaC surface export than aspartate (FIGS. 3C and D). On the other hand, SiaC_{+2KAEVE+6} behaved as SiaC_{+2KKEVE+6}, with very little protein transported to the surface (FIG. 9C). This result highlighted the fact that, although the introduced peptide motif is overall negatively charged, the position of the positively charged amino acid (K at position +3) appears critical for proper surface localization.

To further validate this point, the Inventors constructed an additional hybrid protein by replacing amino acids 18 to 22 from SiaC by amino acids 23 to 27 of MucG (SiaC_{+2KEVEE+6}), shifting the added MucG peptide by one amino acid as compared to SiaC_{+2KKEVE+6}. This thus results in a signal peptide with only one positively charged residue but with K at position +2 rather than +3 (FIG. 9A). Similar to the SiaC_{+2KAEVE+6} construct and in good agreement with our previous results, this construct only localized at the cell surface of 47.9±1.9% of labeled cells (FIG. 9C). Additionally, the fluorescent intensity was low, confirming a positional effect of the lysine residue on surface transport.

Taken together, the Inventors' data with the MuG LES in SiaC further strengthen the previously obtained results with the consensus LES in SiaC, namely the compositional as well as positional requirements of the C. canimorsus LES.

### Experiment 9: Investigation of the LES in the Model Surface Exposed Lipoprotein MucG

The Inventors next wanted to analyze the MucG LES in its native background, prompting them to systematically substitute residues 22 to 29 by alanines in the wt MucG protein (FIG. 10A). After verifying that all mutant proteins were expressed (FIG. 10B), they monitored the surface exposure of the MucG variants by flow cytometry (FIG. 100). Alanine substitution of K22, V25 and E27 did not significantly alter surface exposition of MucG, while mutation of K23, E24, E26, E28 or P29A resulted in a 25 to 50% decrease of exposition. None of the single mutations completely abolished surface localization, suggesting that the MucG motif is redundant, presumably due to the presence of two lysines and four glutamates. The mutation of one of those residues could therefore be compensated by the presence of another one in close proximity. Mutation of K22 did not alter surface exposure, which is in agreement with their previous data obtained with SiaC and the fact that the residues at position +2 are not highly conserved in C. canimorsus surface lipoproteins (FIGS. 2B and C). Not surprisingly either, the V25A substitution did not alter MucG surface exposition, indicating that this residue is likely not playing any role in the MucG LES sequence. This result also agrees with the previously obtained data with SiaC, where surface exposure of the protein was achieved without a valine residue in the added consensus sequence (FIGS. 3C and D).

Since the MucG LES is redundant, the Inventors performed a second set of alanine substitutions by mutating several residues simultaneously (FIG. 11A). After having checked the correct expression of all constructs (FIG. 11B), the Inventors analyzed their surface localization by flow cytometry (FIG. 11C). Expectedly, substitution of the two lysine residues (MucG_{+2AAEVEEE+8}) led to MucG surface exposure in only 23.1±4.5% of the cells (FIG. 100). Additionally, the fluorescent intensity in this subset of cells was markedly decreased as compared to the wt strain (23.8%), indicating that the efficiency of the transport was also strongly affected in this subpopulation. This is in good agreement with their previous data showing that a +2 serine or a +3 lysine is required for surface export.

The same approach was used to investigate the role of the negatively charged residues (MucG_{+2KKAAAAA+8}, MucG_{+2KKAAAEE+8} and MucG_{+2KKEVAAA+8} mutations) (FIG. 11A). While MucG_{+2KKAAAEE+8} and MucG_{+2KKEVAAA+8} were surface exposed in all analyzed cells, their abundance at the surface was reduced, as reflected by a 50% decrease of fluorescent intensity (FIG. 11C). On the other hand, MucG_{+2KKAAAAA+8} was surface localized in only 41.9±6.9% of the cells (FIG. 11C) and the fluorescent intensity in this subpopulation was decreased as compared to the wt strain (24.5%). This confirmed that the negatively charged residues are critical for the surface localization of MucG even if their role seems somewhat less important than what was observed with SiaC.

By combining the data obtained from single and multiple alanine substitutions, the minimal LES for optimal MucG surface exposure appears to be X-K-(D/E)₃ (SEQ ID NO:40-47) downstream from the +1 cysteine, exactly as deduced from the analysis with SiaC.

### Experiment 10: Arginine can Functionally Replace Lysine in the MucG LES

In the Inventors' initial in silico analysis, the lysine located at position +3 was the most conserved residues in C. canimorsus surface exposed lipoproteins (FIGS. 2B and C). Surprisingly however, point mutation of this residue did not affect surface exposure of SiaC unless the +2 residue was also mutated (FIGS. 3C and D). In order to clarify whether the high conservation of lysine was linked to the nature of the amino acid itself or only to its charge, the Inventors replaced the lysine residues in the MucG LES by arginines residues (FIG. 12A). The expression of the resulting constructs, MucG_{+2RREVEEE+8}, MucG_{+2RAEVEEE+8} and MucG_{+2AREVEEE+8}, was then confirmed by western blot (FIG. 12B). Interestingly, substitution of both lysines by arginines led to a clear surface localization of MucG_{+2RREVEEE+8}, although slightly lower as in the wt strain (FIG. 12C). This is likely explained by the fact that arginine at position +3 is only rarely found in C. canimorsus surface lipoproteins. This also indicated that it is indeed the charge of the amino acid rather than the amino acid itself that is important for surface targeting. Surprisingly, MucG_{+2RAEVEEE+8} and MucG_{+2AREVEEE+8} were also both surface exposed, 22-RAEVEEE-28 (SEQ ID NO: 61) being even more potent than the wt sequence for MucG export (FIG. 12C). On the other hand, MucG_{+2AREVEEE+8} was less efficiently transported (FIG. 12C).

Taken together, these data show that the charge rather the nature of the amino acid in position +2 or +3 is involved in MucG surface exposure.

TABLE S1
Bacterial strains used in this study

| Strain | Genotype and/or description |
|---|---|
| Top10 | F- mcrA Δ(mrr-hsdRMS-mcrBC) φ80lacZΔM15 ΔlacX74 recA1 |
| | araD139 Δ(araleu)7697 galU galK rpsL endA1 nupG; Sm^{r} |
| | (obtained from Invitrogen) |
| Cc5 | Wild type (BCCM-LMG 28512) |
| ΔsiaC | Replacement of Ccan_04790 by ermF; Em^{r} |
| ΔmucG | Replacement of Ccan_17430 by ermF; Em^{r} |

TABLE S2
Plasmids used in this study

| Plasmid | Description |
|---|---|
| pMM47.A | ColE1 ori; (pCC7 ori); Ap^{r}; (Cfx^{r}). E. coli-C. canimorsus expression shuttle |
| | plasmid with ermF promoter |
| pPM5 | ColE1 ori; (pCC7 ori); Ap^{r}; (Cfx^{r}). E. coli-C. canimorsus expression shuttle |
| | plasmid with ompA promoter |
| pFL43 | Full lenght mucG with a C-terminal HA tag amplified with primers 7182/7625 |
| | and cloned into pPM5 using NcoI/XhoI restriction sites |
| pFL44 | Full lenght mucG C21G with a C-terminal HA tag amplified with primers |
| | 7259/7625 and cloned into pPM5 using NcoI/XhoI restriction sites |
| pFL71 | Full length mucG K22A with a C-terminal HA tag amplified with primers |
| | 7182/7487 and 7486/7625 and cloned into pPM5 using NcoI/XhoI restriction |
| | sites |
| pFL72 | Full length mucG K23A with a C-terminal HA tag amplified with primers |
| | 7182/7489 and 7488/7625 and cloned into pPM5 using NcoI/XhoI restriction |
| | sites |
| pFL73 | Full length mucG E24A with a C-terminal HA tag amplified with primers |
| | 7182/7491 and 7490/7625 and cloned into pPM5 using NcoI/XhoI restriction |
| | sites |
| pFL74 | Full length mucG V25A with a C-terminal HA tag amplified with primers |
| | 7182/7493 and 7492/7625 and cloned into pPM5 using NcoI/XhoI restriction |
| | sites |
| pFL75 | Full length mucG E26A with a C-terminal HA tag amplified with primers |
| | 7182/7495 and 7494/7625 and cloned into pPM5 using NcoI/XhoI restriction |
| | sites |
| pFL76 | Full length mucG E27A with a C-terminal HA tag amplified with primers |
| | 7182/8048 and 8047/7625 and cloned into pPM5 using NcoI/XhoI restriction |
| | sites |
| pFL77 | Full length mucG E28A with a C-terminal HA tag amplified with primers |
| | 7182/8050 and 8049/7625 and cloned into pPM5 using NcoI/XhoI restriction |
| | sites |
| pFL78 | Full length mucG P29A with a C-terminal HA tag amplified with primers |
| | 7182/7570 and 7569/7625 and cloned into pPM5 using NcoI/XhoI restriction |
| | sites |
| pFL79 | Full length mucG with a C-terminal HA tag amplified with primers 7182/7510 |
| | and 7509/7625 and cloned into pPM5 using NcoI/XhoI restriction sites. |
| | Replacement of aa 22-28 by AAEVEEE |
| pFL80 | Full length mucG with a C-terminal HA tag amplified with primers 7182/7512 |
| | and 7511/7625 and cloned into pPM5 using NcoI/XhoI restriction sites. |
| | Replacement of aa 22-28 by KKAAAEE |
| pFL81 | Full length mucG with a C-terminal HA tag amplified with primers 7182/7514 |
| | and 7513/7625 and cloned into pPM5 using NcoI/XhoI restriction sites. |
| | Replacement of aa 22-28 by KKEVAAA |
| pFL84 | Full length mucG with a C-terminal HA tag amplified with primers 7182/7899 |
| | and 7898/7625 and cloned into pPM5 using NcoI/XhoI restriction sites. |
| | Replacement of aa 22-28 by KKAAAAA |
| pFL97 | Full length mucG with a C-terminal HA tag amplified with primers 7182/7897 |
| | and 7896/7625 and cloned into pPM5 using NcoI/XhoI restriction sites. |
| | Replacement of aa 22-28 by RREVEEE |
| pFL98 | Full length mucG with a C-terminal HA tag amplified with primers 7182/7893 |
| | and 7892/7625 and cloned into pPM5 using NcoI/XhoI restriction sites. |
| | Replacement of aa 22-28 by RAEVEEE |
| pFL99 | Full length mucG with a C-terminal HA tag amplified with primers 7182/7895 |
| | and 7894/7625 and cloned into pPM5 using NcoI/XhoI restriction sites. |
| | Replacement of aa 22-28 by AREVEEE |
| pFL117 | Full lenght siaC amplified with primers 4159 and 7696 and cloned into |
| | pMM47.A using NcoI/XhoI restriction sites |
| pFL118 | Full lenght siaC C17G amplified with primers 5545 and 7696 and cloned into |
| | pMM47.A using NcoI/XhoI restriction sites |
| pFL132 | Full lenght siaC amplified with primers 4159/8017 and 8016/7696 and cloned |
| | into pMM47.A using NcoI/XhoI restriction sites. Replacement of aa 18-22 by |
| | KKEVE |
| pFL133 | Full lenght siaC amplified with primers 4159/8054 and 8052/7696 and cloned |
| | into pMM47.A using NcoI/XhoI restriction sites. Replacement of aa 18-22 by |
| | KKEVEE |
| pFL134 | Full lenght siaC amplified with primers 4159/7972 and 7971/7696 and cloned |
| | into pMM47.A using NcoI/XhoI restriction sites. Replacement of aa 18-22 by |
| | KKEVEEE |
| pFL140 | Full length siaC amplified with primers 4159/8029 and 8028/7696 and cloned |
| | into pMM47.A using NcoI/XhoI restriction sites. Replacement of aa 18-22 by |
| | AKEVE |
| pFL141 | Full length siaC amplified with primers 4159/8031 and 8030/7696 and cloned |
| | into pMM47.A using NcoI/XhoI restriction sites. Replacement of aa 18-22 by |
| | KAEVE |
| pFL142 | Full length siaC amplified with primers 4159/8082 and 8081/7696 and cloned |
| | into pMM47.A using NcoI/XhoI restriction sites. Replacement of aa 18-22 by |
| | KEVEE |
| pFL143 | Full lenght siaC amplified with primers 4159/8058 and 8057/7696 and cloned |
| | into pMM47.A using NcoI/XhoI restriction sites. Replacement of aa 18-22 by |
| | QKDDE |
| pFL144 | Full lenght siaC amplified with primers 4159/8086 and 8085/7696 and cloned |
| | into pMM47.A using NcoI/XhoI restriction sites. Replacement of aa 18-22 by |
| | AKDDE |
| pFL145 | Full lenght siaC amplified with primers 4159/8084 and 8083/7696 and cloned |
| | into pMM47.A using NcoI/XhoI restriction sites. Replacement of aa 18-22 by |
| | AKDDA |
| pFL146 | Full lenght siaC amplified with primers 4159/8153 and 8152/7696 and cloned |
| | into pMM47.A using NcoI/XhoI restriction sites. Replacement of aa 18-22 by |
| | AKEEA |
| pFL147 | Full lenght siaC amplified with primers 4159/8149 and 8148/7696 and cloned |
| | into pMM47.A using NcoI/XhoI restriction sites. Replacement of aa 18-22 by |
| | AKDAA |
| pFL148 | Full lenght siaC amplified with primers 4159/8151 and 8150/7696 and cloned |
| | into pMM47.A using NcoI/XhoI restriction sites. Replacement of aa 18-22 by |
| | AKEAA |
| pFL149 | Full lenght siaC amplified with primers 4159/8157 and 8156/7696 and cloned |
| | into pMM47.A using NcoI/XhoI restriction sites. Replacement of aa 18-22 by |
| | AAKDD |
| pFL150 | Full lenght siaC amplified with primers 4159/8159 and 8158/7696 and cloned |
| | into pMM47.A using NcoI/XhoI restriction sites. Replacement of aa 18-22 by |
| | AAAKDD |
| pFL151 | Full lenght siaC amplified with primers 4159/8161 and 8160/7696 and cloned |
| | into pMM47.A using NcoI/XhoI restriction sites. Replacement of aa 18-22 by |
| | AAAAKDD |
| pFL152 | Full lenght siaC amplified with primers 4159/8169 and 8168/7696 and cloned |
| | into pMM47.A using NcoI/XhoI restriction sites. Replacement of aa 18-22 by |
| | KDDAA |
| pFL153 | Full lenght siaC amplified with primers 4159/8165 and 8164/7696 and cloned |
| | into pMM47.A using NcoI/XhoI restriction sites. Replacement of aa 18-22 by |
| | QADDE |
| pFL154 | Full lenght siaC amplified with primers 4159/8167 and 8166/7696 and cloned |
| | into pMM47.A using NcoI/XhoI restriction sites. Replacement of aa 18-22 by |
| | AADDA |
| pFL155 | Full lenght siaC amplified with primers 4159/8164 and 8163/7696 and cloned |
| | into pMM47.A using NcoI/XhoI restriction sites. Replacement of aa 18-22 by |
| | SDDFE |
| pFL156 | Full lenght siaC amplified with primers 4159/8173 and 8172/7696 and cloned |
| | into pMM47.A using NcoI/XhoI restriction sites. Replacement of aa 18-22 by |
| | SDDDD |

^{a} Selection markers for C. canimorsus are in between brackets

TABLE S3
Oligonucleotides used in this study

| | | SEQ |
|---|---|---|
| Ref. | Sequence 5′-3′ | ID NO: |
| 4159 | cataccatgggaaatcgaattttttatctt | 72 |
| | (restriction: NcoI) | |
| 5545 | catgccatgggaaatcgaattttttatcttttatt | 73 |
| | cgcttttgttcttttgtcggctggtggaagccaaa | |
| | aaaacg (restriction: NcoI) | |
| 7182 | ggccatggggaaaaaaatagtatccattagc | 74 |
| | (restriction: NcoI) | |
| 7259 | ggccatggggaaaaaaatagtatccattagcttat | 75 |
| | ttttccttatctcagcaactatttggttagccggt | |
| | aaaaaggaag (restriction: NcoI) | |
| 7486 | tggttagcctgtgcaaaggaagttgaagaagaacc | 76 |
| 7487 | ggttcttcttcaacttcctttgcacaggctaacca | 77 |
| 7488 | ttagcctgtaaagcggaagttgaagaagaaccttt | 78 |
| | tc | |
| 7489 | gaaaaggttcttcttcaacttccgctttacaggct | 79 |
| | aa | |
| 7490 | gcctgtaaaaaggcagttgaagaagaaccttttct | 80 |
| | aac | |
| 7491 | gttagaaaaggttcttcttcaactgcctttttaca | 81 |
| | ggc | |
| 7492 | tgtaaaaaggaagctgaagaagaaccttttctaac | 82 |
| 7493 | gttagaaaaggttcttcttcagcttcctttttaca | 83 |
| 7494 | aaaaaggaagttgcagaagaaccttttctaacaat | 84 |
| | ag | |
| 7495 | ctattgttagaaaaggttcttctgcaacttccttt | 85 |
| | tt | |
| 7509 | tggttagcctgtgcagcggaagttgaagaagaacc | 86 |
| 7510 | ggttcttcttcaacttccgctgcacaggctaacca | 87 |
| 7511 | gcctgtaaaaaggcagctgcagaagaaccttttct | 88 |
| | aac | |
| 7512 | gttagaaaaggttcttctgcagctgcctttttaca | 89 |
| | ggc | |
| 7513 | aaaaaggaagttgcagcagcaccttttctaacaat | 90 |
| | ag | |
| 7514 | ctattgttagaaaaggtgctgctgcaacttccttt | 91 |
| | tt | |
| 7569 | gttgaagaagaagcttttctaacaatagaagaaaa | 92 |
| | aacc | |
| 7570 | ggttttttcttctattgttagaaaagcttcttctt | 93 |
| | caac | |
| 7625 | ggctcgagctaagcgtaatctggaacatcgtatgg | 94 |
| | gtaaaacgtaacttgagttctc | |
| | (restriction: XhoI) | |
| 7696 | ggctcgagttagttcttgataaattcctcaactgg | 95 |
| | (restriction: XhoI) | |
| 7892 | tggttagcctgtagagcggaagttgaagaagaacc | 96 |
| | ttttc | |
| 7893 | gaaaaggttcttcttcaacttccgctctacaggct | 97 |
| | aacca | |
| 7894 | ttagcctgtgcaagagaagttgaagaagaaccttt | 98 |
| | tc | |
| 7895 | gaaaaggttcttcttcaacttctcttgcacaggct | 99 |
| | aa | |
| 7896 | tggttagcctgtagaagagaagttgaagaagaacc | 100 |
| | ttttc | |
| 7897 | gaaaaggttcttcttcaacttctcttctacaggct | 101 |
| | aacca | |
| 7898 | gcagctgcagcggctccttttctaacaatagaaga | 102 |
| | aaaaacc | |
| 7899 | agccgctgcagctgcctttttacaggctaaccaaa | 103 |
| | tagttgc | |
| 7971 | aaaaaggaagttgaagaagaagtaatcggcggagg | 104 |
| | cgaatttacacaacccg | |
| 7972 | ttcttcttcaacttcctttttacaagccgacaaaa | 105 |
| | gaacaaaagcg | |
| 8016 | aaaaaggaagttgaagtaatcggcggaggcgaatt | 106 |
| | tacacaacccg | |
| 8017 | ttcaacttcctttttacaagccgacaaaagaacaa | 107 |
| | aagcg | |
| 8028 | gcaaaggaagttgaagtaatcggcggaggcgaatt | 108 |
| | tacacaacccg | |
| 8029 | ttcaacttcctttgcacaagccgacaaaagaacaa | 109 |
| | aagcg | |
| 8030 | aaagcggaagttgaagtaatcggcggaggcgaatt | 110 |
| | tacacaacccg | |
| 8031 | ttcaacttccgctttacaagccgacaaaagaacaa | 111 |
| | aagcg | |
| 8047 | aggaagttgaagcagaaccttttctaacaatagaa | 112 |
| | gaaaaaacc | |
| 8048 | gaaaaggttctgcttcaacttcctttttacaggct | 113 |
| | aacc | |
| 8049 | ggaagttgaagaagcaccttttctaacaatagaag | 114 |
| | aaaaaacc | |
| 8050 | gaaaaggtgcttcttcaacttcctttttacaggct | 115 |
| | aaccaaatagttg | |
| 8081 | aaggaagttgaagaagtaatcggcggaggcgaatt | 116 |
| | tacacaacccg | |
| 8082 | ttcttcaacttccttacaagccgacaaaagaacaa | 117 |
| | aagcg | |
| 8052 | aaaaaggaagttgaagaagtaatcggcggaggcga | 118 |
| | atttacacaacccg | |
| 8054 | ttcttcaacttcctttttacaagccgacaaaagaa | 119 |
| | caaaagcg | |
| 8057 | caaaaggacgatgaagtaatcggcggaggcgaatt | 120 |
| | tacacaacccg | |
| 8058 | ttcatcgtccttttgacaagccgacaaaagaacaa | 121 |
| | aagcg | |
| 8083 | gcaaaggacgatgcagtaatcggcggaggcgaatt | 122 |
| | tacacaacccg | |
| 8084 | tgcatcgtcctttgcacaagccgacaaaagaacaa | 123 |
| | aagcg | |
| 8085 | gcaaaggacgatgaagtaatcggcggaggcgaatt | 124 |
| | tacacaacccg | |
| 8086 | ttcatcgtcctttgcacaagccgacaaaagaacaa | 125 |
| | aagcg | |
| 8148 | gcaaaggacgctgcagtaatcggcggaggcgaatt | 126 |
| | tacacaacccg | |
| 8149 | tgcagcgtcctttgcacaagccgacaaaagaacaa | 127 |
| | aagcg | |
| 8150 | gcaaaggaagctgcagtaatcggcggaggcgaatt | 128 |
| | tacacaacccg | |
| 8151 | tgcagcttcctttgcacaagccgacaaaagaacaa | 129 |
| | aagcg | |
| 8152 | gcaaaggaagaggcagtaatcggcggaggcgaatt | 130 |
| | tacacaacccg | |
| 8153 | tgcctcttcctttgcacaagccgacaaaagaacaa | 131 |
| | aagcg | |
| 8156 | gctgcaaaggacgatgtaatcggcggaggcgaatt | 132 |
| | tacacaacccg | |
| 8157 | atcgtcctttgcagcacaagccgacaaaagaacaa | 133 |
| | aagcg | |
| 8158 | gcagctgcaaaggacgatgtaatcggcggaggcga | 134 |
| | atttacacaacccg | |
| 8159 | atcgtcctttgcagctgcacaagccgacaaaagaa | 135 |
| | caaaagcg | |
| 8160 | gccgcagctgcaaaggacgatgtaatcggcggagg | 136 |
| | cgaatttacacaacccg | |
| 8161 | atcgtcctttgcagctgcggcacaagccgacaaaa | 137 |
| | gaacaaaagcg | |
| 8162 | tctgatgacttcgaagtaatcggcggaggcgaatt | 138 |
| | tacacaacccg | |
| 8163 | ttcgaagtcatcagaacaagccgacaaaagaacaa | 139 |
| | aagcg | |
| 8164 | caagcggacgatgaagtaatcggcggaggcgaatt | 140 |
| | tacacaacccg | |
| 8165 | ttcatcgtccgcttgacaagccgacaaaagaacaa | 141 |
| | aagcg | |
| 8166 | gcagctgacgatgcagtaatcggcggaggcgaatt | 142 |
| | tacacaacccg | |
| 8167 | tgcatcgtcagctgcacaagccgacaaaagaacaa | 143 |
| | aagcg | |
| 8168 | aaggacgatgcagctgtaatcggcggaggcgaatt | 144 |
| | tacacaacccg | |
| 8169 | agctgcatcgtccttacaagccgacaaaagaacaa | 145 |
| | aagcg | |
| 8172 | agtgatgacgacgatgtaatcggcggaggcgaatt | 146 |
| | tacacaacccg | |
| 8173 | atcgtcgtcatcactacaagccgacaaaagaacaa | 147 |
| | aagcg | |

^{a}Restriction sites are underlined

TABLE S4
C. canimorsus 5 surface exposed lipoproteins

| Uniprot | | | SPII cleavage | % of |
|---|---|---|---|---|
| Accession | ORF name | Annotation | site^{c} | surfome^{d} |
| F9YPG1 | Ccan_00120 | Uncharacterized protein | 22-23 | 8.35 |
| F9YPG2 | Ccan_00130 | Uncharacterized protein | 19-20 | 4.25 |
| F9YPJ0 | Ccan_00410 | Uncharacterized protein | 18-19 | 0.23 |
| F9YPJ1 | Ccan_00420 | Uncharacterized protein | 17-18 | 0.32 |
| F9YPJ2 | Ccan_00430 | Uncharacterized protein | 20-21 | 0.27 |
| F9YPJ3 | Ccan_00440 | Uncharacterized protein | 19-20 | 0.14 |
| F9YPV6 | Ccan_00790 | Uncharacterized protein | 19-20 | 12.8 |
| F9YPV7 | Ccan_00800 | Tetanolysin O | 19-20 | 0.58 |
| F9YPV8 | Ccan_00810 | Uncharacterized protein | 12-13 | 0.46 |
| F9YQU8 | Ccan_02630 | UPF0312 protein | 19-20 | 3.63 |
| F9YRN1 | Ccan_03880 | TvBspA-like-625 | 20-21 | 1.24 |
| F9YS71 | Ccan_05040 | Glycosyl hydrolase family 109 | 26-27 | / |
| | | protein 5 (EC 3.2.1.49) | | |
| F9YS78 | Ccan_05110 | Uncharacterized protein | 18-19 | 0.69 |
| F9YSN4 | Ccan_05870 | Carboxyl-terminal-processing | 16-17 | 1.02 |
| | | protease (EC 3.4.21.102) | | |
| F9YT40 | Ccan_06620 | Thiol-activated cytolysin | 21-22 | 1.37 |
| F9YTK6 | Ccan_07500 | Uncharacterized protein | 16-17 | 0.45 |
| F9YTK7 | Ccan_07510 | Uncharacterized protein | 15-16 | 0.2 |
| F9YTY4 | Ccan_08000 | Uncharacterized protein | 19-20 | / |
| F9YUD4 | Ccan_08710 | GpdD | 16-17 | 3.99 |
| F9YUD5 | Ccan_08720 | GpdG | 20-21 | 3.43 |
| F9YUD6 | Ccan_08730 | GpdE | 16-17 | 1.28 |
| F9YUD7 | Ccan_08740 | GpdF | 17-18 | 3.25 |
| F9YUS3 | Ccan_09300 | Thioredoxin family protein (EC | 16-17 | / |
| | | 1.8.1.8) | | |
| F9YUW3 | Ccan_09700 | Peptidyl-prolyl cis-trans | 19-20 | 0.71 |
| | | isomerase (EC 5.2.1.8) | | |
| F9YVS5 | Ccan_11230 | Uncharacterized protein | 17-18 | 0.17 |
| F9YVT2 | Ccan_11300 | Uncharacterized protein | 17-18 | 1.11 |
| F9YPL2 | Ccan_12420 | Uncharacterized protein | 18-19 | 2.57 |
| F9YQG8 | Ccan_13910 | Uncharacterized protein | 21-22 | 0.27 |
| F9YQN5 | Ccan_14580 | Internalin-J (EC 3.2.1.83) | 23-24 | 0.23 |
| F9YSD4 | Ccan_17430^{a} | MucG mucinase | 20-21 | 1.29 |
| F9YSD5 | Ccan_17440 | MucE | 18-19 | 8.99 |
| F9YTL6 | Ccan_19450 | Uncharacterized protein | 18-19 | 5.15 |
| F9YTT1 | Ccan_20100 | Uncharacterized protein | 19-20 | / |
| F9YTT2 | Ccan_20110 | Uncharacterized protein | 20-21 | 1.64 |
| F9YTT3 | Ccan_20120^{b} | Uncharacterized protein | 20-21 | 2.08 |
| F9YUN2 | Ccan_21530 | Uncharacterized protein | 23-24 | / |
| F9YUN4 | Cean_21550 | Uncharacterized protein | 23-24 | 0.09 |
| F9YUP2 | Ccan_21630 | Uncharacterized protein | 24-25 | 11.3 |
| F9YV08 | Ccan_22020 | Uncharacterized protein | 17-18 | 0.03 |
| F9YV37 | Ccan_22310 | Uncharacterized protein | 21-22 | 0.17 |
| F9YV38 | Ccan_22320 | Uncharacterized protein | 20-21 | 0.19 |
| F9YVG4 | Ccan_22830 | Uncharacterized protein | 16-17 | 0.12 |
| F9YVZ6 | Ccan_23850 | Uncharacterized protein | 17-18 | / |
| | | | Total | 84.06 |

^{a}Using the annotated translational start site Ccan_17430 is predicted to be a cytoplasmic protein, but if translation begins at an AUG 13 codons downstream then it is predicted to be a lipoprotein
^{b}Using the annotated translational start site Ccan_20120 is predicted to be a cytoplasmic protein, but if translation begins at an AUG 18 codons downstream then it is predicted to be a lipoprotein.
^{c}SPII cleavage site predicted by the LipoP software; numbers indicate the position of the last amino acid of the signal peptide and the position of the +1 cysteine.
^{d}Quantitative contribution to surfome composition, expressed in percentage, as described in.(Manfredi, P., et al., The genome and surface proteome of Capnocytophaga canimorsus reveal a key role of glycan foraging systems in host glycoproteins deglycosylation. Mol Microbiol, 2011. 81 (4): p. 1050-60)
‘/’ stands for not quantified.

TABLE S5
C. canimorsus 5 periplasmic outer membrane lipoproteins

| Uniprot | | | SPII cleavage |
|---|---|---|---|
| Accession | ORF name | Annotation | site^{a} |
| F9YQA5 | Ccan_01510 | Putative Subtilisin (EC 3.4.21.62) | 18-19 |
| F9YQE9 | Ccan_01950 | Uncharacterized protein | 19-20 |
| F9YRN0 | Ccan_03870 | Surface antigen BspA | 20-21 |
| F9YS48 | Ccan_04790 | Neuraminidase | 16-17 |
| F9YT17 | Ccan_06390 | Membrane or secreted protein | 15-16 |
| F9YT18 | Ccan_06400 | Inner membrane lipoprotein yiaD | 16-17 |
| F9YT35 | Ccan_06570 | Uncharacterized protein | 19-20 |
| F9YT36 | Ccan_06580 | Uncharacterized protein | 22-23 |
| F9YV81 | Ccan_10100 | Uncharacterized protein | 19-20 |
| F9YQI1 | Ccan_14040 | Uncharacterized protein | 16-17 |
| F9YQL3 | Ccan_14360 | Uncharacterized protein | 32-33 |
| F9YQM4 | Ccan_14470 | OmpA/MotB C-terminal like outer | 17-18 |
| | | membrane protein | |
| F9YSV1 | Ccan_18300 | Uncharacterized protein | 25-26 |
| F9YTS3 | Ccan_20020 | Uncharacterized protein | 20-21 |
| F9YV05 | Ccan_21990 | Uncharacterized protein | 16-17 |
| F9YV31 | Ccan_22250 | Tvall (EC 3.2.1.1) | 36-37 |
| F9YV59 | Ccan_22530 | Uncharacterized protein | 20-21 |

^{a}SPII cleavage site predicted by the LipoP software; numbers indicate the position of the last amino acid of the signal peptide and the position of the +1 cysteine.

TABLE S6
B. fragilis NCTC 9343 proteinase K sensitive surface exposed lipoproteins

| Uniprot | | | SPII cleavage |
|---|---|---|---|
| Accession | ORF name | Annotation | site^{c} |
| Q5L9H5 | BF9343_3471 | Uncharacterized protein | 21-22 |
| Q5LAW1 | BF9343_2981 | Putative lipoprotein | 22-23 |
| Q5LAN4 | BF9343_3058 | Putative lipoprotein | 18-19 |
| Q5LBW6 | BF9343_2621 | Putative lipoprotein | 22-23 |
| Q5LFL5 | BF9343_1297 | Uncharacterized protein | 18-19 |
| Q5LFL6 | BF9343_1296 | Uncharacterized protein | 20-21 |
| Q5LF14 | BF9343_1504 | Uncharacterized protein | 25-26 |
| Q5LDF5 | BF9343_2074 | Putative exported protein | 21-22 |
| Q5LFR2 | BF9343_1250 | Uncharacterized protein | 22-23 |
| Q5LGH3 | BF9343_0985 | Conserved hypothetical lipoprotein | 24-25 |
| Q5L8V3 | BF9343_3698 | Putative exported protein | 20-21 |
| Q5L9U0 | BF9343_3356 | Putative lipoprotein | 23-24 |
| Q5LF13 | BF9343_1505 | Uncharacterized protein | 37-38 |
| Q5LDF3 | BF9343_2076 | Putative lipoprotein | 25-26 |
| Q5LAV1 | BF9343_2991 | Putative exported protein | 19-20 |
| Q5LFL7 | BF9343_1295^{a} | Uncharacterized protein | 24-25 |
| Q5CZE9 | BF9343_p20^{b} | Uncharacterized protein | 18-19 |
| Q5L9U1 | BF9343_3355 | Uncharacterized protein | 29-30 |
| Q5L7N0 | BF9343_4139 | Putative outer membrane protein | 28-29 |
| Q5LGX6 | BF9343_0829 | Possible outer membrane protein | 16-17 |
| Q5LDF1 | BF9343_2078 | Conserved hypothetical lipoprotein | 21-22 |
| Q5L7M9 | BF9343_4140 | Uncharacterized protein | 25-26 |

^{a}The translational start site of BF9343_1295 was moved 15 codons downstream, resulting in a predicted lipoprotein.
^{b}The translational start site of BF9343_p20 was moved 38 codons downstream, resulting in a predicted lipoprotein.
^{c}SPII cleavage site predicted by the LipoP software; numbers indicate the position of the last amino acid of the signal peptide and the position of the +1 cysteine.

TABLE S7
F. johnsoniae UW101 SusD-like lipoproteins

| Uniprot | | | SPII cleavage |
|---|---|---|---|
| Accession | ORF name | Annotation | site^{a} |
| A5FNK0 | Fjoh_0184 | RagB/SusD domain protein | 22-23 |
| A5FMX2 | Fjoh_0404 | RagB/SusD domain protein | 17-18 |
| A5FM74 | Fjoh_0666 | RagB/SusD domain protein | 19-20 |
| A5FLV9 | Fjoh_0781 | RagB/SusD domain protein | 26-27 |
| A5FKM3 | Fjoh_1212 | RagB/SusD domain protein | 19-20 |
| A5FK32 | Fjoh_1406 | RagB/SusD domain protein | 24-25 |
| A5FJL9 | Fjoh_1561 | RagB/SusD domain protein | 21-22 |
| A5FIL9 | Fjoh_1925 | RagB/SusD domain protein | 19-20 |
| A5FIC6 | Fjoh_2009 | RagB/SusD domain protein | 20-21 |
| A5FIB2 | Fjoh_2021 | RagB/SusD domain protein | 18-19 |
| A5FI96 | Fjoh_2044 | RagB/SusD domain protein | 22-23 |
| A5FI68 | Fjoh_2078 | RagB/SusD domain protein | 20-21 |
| A5FH57 | Fjoh_2432 | RagB/SusD domain protein | 21-22 |
| A5FGD1 | Fjoh_2712 | RagB/SusD domain protein | 21-22 |
| A5FFU9 | Fjoh_2893 | RagB/SusD domain protein | 18-19 |
| A5FFG2 | Fjoh_3036 | RagB/SusD domain protein | 34-35 |
| A5FF76 | Fjoh_3126 | RagB/SusD domain protein | 20-21 |
| A5FEV7 | Fjoh_3250 | RagB/SusD domain protein | 19-20 |
| A5FEL9 | Fjoh_3338 | RagB/SusD domain protein | 24-25 |
| A5FE35 | Fjoh_3524 | RagB/SusD domain protein | 17-18 |
| A5FDZ2 | Fjoh_3557 | RagB/SusD domain protein | 27-28 |
| A5FDB1 | Fjoh_3801 | RagB/SusD domain protein | 18-19 |
| A5FD47 | Fjoh_3864 | RagB/SusD domain protein | 21-22 |
| A5FD39 | Fjoh_3870 | RagB/SusD domain protein | 20-21 |
| A5FD24 | Fjoh_3881 | RagB/SusD domain protein | 21-22 |
| A5FCW3 | Fjoh_3944 | RagB/SusD domain protein | 19-20 |
| A5FCG9 | Fjoh_4094 | RagB/SusD domain protein | 20-21 |
| A5FCA0 | Fjoh_4168 | RagB/SusD domain protein | 23-24 |
| A5FC59 | Fjoh_4195 | RagB/SusD domain protein | 17-18 |
| A5FC33 | Fjoh_4233 | RagB/SusD domain protein | 21-22 |
| A5FC07 | Fjoh_4254 | RagB/SusD domain protein | 17-18 |
| A5FBT2 | Fjoh_4328 | RagB/SusD domain protein | 18-19 |
| A5FBM9 | Fjoh_4374 | RagB/SusD domain protein | 34-35 |
| A5FBI4 | Fjoh_4433 | RagB/SusD domain protein | 20-21 |
| A5FBC7 | Fjoh_4490 | RagB/SusD domain protein | 24-25 |
| A5FBC2 | Fjoh_4499 | RagB/SusD domain protein | 17-18 |
| A5FB66 | Fjoh_4558 | RagB/SusD domain protein | 20-21 |
| A5FB55 | Fjoh_4561 | RagB/SusD domain protein | 18-19 |
| A5FAX8 | Fjoh_4646 | RagB/SusD domain protein | 17-19 |
| A5FAV5 | Fjoh_4672 | RagB/SusD domain protein | 19-20 |
| A5FAF6 | Fjoh_4815 | RagB/SusD domain protein | 25-26 |
| A5FA21 | Fjoh_4950 | RagB/SusD domain protein | 24-25 |

^{a}SPII cleavage site predicted by the LipoP software; numbers indicate the position of the last amino acid of the signal peptide and the position of the +1 cysteine.

1. A polypeptide precursor comprising
(a) an N-terminal signal peptide of a lipoprotein of Gram-negative bacteria comprising a lipobox motif located at the very end of the C-terminus of said signal peptide, wherein said lipobox motif consists of the amino acid sequence L(S/A)(A/G)C and is specifically recognizable by a signal peptidase type II;
(b) a lipoprotein export signal comprising an amino acid sequence according to any one of the following consensus sequences:
XJZZ, wherein X can be any amino acid, wherein J is selected from the group consisting of K and A, wherein Z is selected from the group consisting of D and E, with the proviso that when J is A, X is Q;
BZZUZ, wherein B is selected from the group consisting of S and T, wherein Z is selected from the group consisting of D and E and wherein U is selected from the group consisting of D, E and F; or
XKEOEE, wherein X and O can be any amino acid, preferably wherein O is V;
wherein said lipoprotein export signal is overall negatively charged and wherein said lipoprotein export signal is located directly adjacent to the C-terminus of said signal peptide;
(c) a polypeptide, wherein said polypeptide is located C-terminally of said signal peptide and said lipoprotein export signal; and
(d) optionally, a protease cleavage site motif, wherein said protease cleavage site motif is different from said lipobox motif and is located C-terminally of said signal peptide and said lipoprotein export signal and N-terminally of said polypeptide;
wherein said signal peptide, said lipoprotein export signal and said polypeptide, do not naturally occur together in a polypeptide sequence.

2. The polypeptide precursor according to claim 1, wherein said N-terminal signal peptide of a lipoprotein of Gram-negative bacteria is the signal peptide of sialidase (siaC) or mucinase (MucG) of C. canimorsus 5.

3. The polypeptide precursor according to claim 1, wherein said lipoprotein export signal is selected from an amino acid sequence according to
any one of SEQ ID NO: 16 to SEQ ID NO:20 or SEQ ID NO: 40 to 47;
any one of SEQ ID NO:1 to SEQ ID NO: 15 or SEQ ID NO: 25 to 39; or
any one of SEQ ID NO:49 to SEQ ID NO:51 or SEQ ID NO:63.

4. A nucleic acid encoding the polypeptide precursor according to claim 1.

5. A recombinant expression vector comprising the nucleic acid according to claim 4, a promoter and transcriptional and translational stop signals, and optionally a selectable marker.

6. A recombinant expression vector comprising
(a) a nucleic acid sequence encoding a signal peptide of a lipoprotein of Gram-negative bacteria wherein said signal peptide comprises a lipobox motif located at the very end of the C-terminus of said signal peptide, wherein said lipobox motif consists of the amino acid sequence L(S/A)(A/G)C and is specifically recognized by a signal peptidase type II;
(b) a nucleic acid sequence encoding a lipoprotein export signal having an amino acid sequence according to any one of the following consensus sequences:
XJZZ, wherein X can be any amino acid, wherein J is selected from the group consisting of K and A, wherein Z is selected from the group consisting of D and E; with the proviso that when J is A, X is Q;
BZZUZ, wherein B is selected from the group consisting of S and T, wherein Z is selected from the group consisting of D and E and wherein U is selected from the group consisting of D, E and F; or
XKEOEE, wherein X and O can be any amino acid, preferably wherein O is V;
wherein said lipoprotein export signal is overall negatively charged and wherein said nucleic acid sequence encoding said lipoprotein export signal is located directly downstream of said nucleic acid sequence encoding said signal peptide;
(c) optionally, a nucleic acid sequence encoding a protease cleavage site motif, wherein said protease cleavage site motif is different from said lipobox motif and is located downstream of said nucleic acid sequence encoding said lipoprotein export signal and said nucleic acid sequence encoding said signal peptide; and
(d) a multiple cloning site, wherein said multiple cloning site is located downstream of said nucleic acid encoding said lipoprotein export signal and said nucleic acid encoding said signal peptide and, optionally downstream of said protease cleavage site motif.

7. The recombinant expression vector according to claim 6, wherein said N-terminal signal peptide of a lipoprotein of Gram-negative bacteria is the signal peptide of sialidase (siaC) or mucinase (MucG) of C. canimorsus 5.

8. The recombinant expression vector according to claim 6, wherein said lipoprotein export signal is selected from an amino acid sequence according to
any one of SEQ ID NO: 16 to SEQ ID NO:20 or SEQ ID NO: 40 to 47;
any one of SEQ ID NO:1 to SEQ ID NO: 15 or SEQ ID NO: 25 to 39; or
any one of SEQ ID NO:49 to SEQ ID NO:51 or SEQ ID NO:63.

9. A recombinant host cell comprising the vector according to claim 5, wherein said host cell is a bacterial cell of the Bacteroidetes phylum.

10. The host cell according to claim 9, wherein said bacterial cell of the Bacteroidetes phylum is Capnocytophaga canimorsus or Flavobacterium johnsoniae.

11. A lipoprotein export signal comprising an amino acid sequence according to one of the following consensus sequences:
XJZZ, wherein X can be any amino acid, wherein J is selected from the group consisting of K and A, wherein Z is selected from the group consisting of D and E; with the proviso that when J is A, X is Q;
BZZUZ, wherein B is selected from the group consisting of S and T, wherein Z is selected from the group consisting of D and E and wherein U is selected from the group consisting of D, E and F; or
XKEOEE, wherein X and O can be any amino acid, preferably wherein O is V;
wherein said lipoprotein export signal is overall negatively charged and wherein said lipoprotein export signal is located directly adjacent to an N-terminal lipid-modified cysteine residue originating from an N-terminal signal peptide of a lipoprotein of Gram-negative bacteria comprising a lipobox motif located at the very end of the C-terminus of said signal peptide, wherein said lipobox motif consists of the amino acid sequence L(S/A)(A/G)C and is specifically recognizable by a signal peptidase type II, for surface exposure of a polypeptide, in a host cell, wherein said polypeptide originates from the same or a different organism than said host cell and wherein said lipoprotein export signal and said polypeptide do not naturally occur together in a polypeptide sequence.

12. The lipoprotein export signal according to claim 11 wherein said N-terminal signal peptide of a lipoprotein of Gram-negative bacteria is the signal peptide of sialidase (siaC) or mucinase (MucG) of C. canimorsus 5.

13. The lipoprotein export signal according to claim 11, wherein said lipoprotein export signal is selected from an amino acid sequence according to
any one of SEQ ID NO: 16 to SEQ ID NO:20 or SEQ ID NO: 40 to 47;
any one of SEQ ID NO:1 to SEQ ID NO: 15 or SEQ ID NO: 25 to 39; or
any one of SEQ ID NO:49 to SEQ ID NO:51 or SEQ ID NO:63.

14. A method of manufacturing a vaccine, for producing antibodies, for biosorption applications, for manufacturing biosensors, for performing bacterial display, for whole-cell based biocatalytic applications or for protein production and purification, the method comprising using the polypeptide precursor according to claim 1 for manufacturing a vaccine, for producing antibodies, for biosorption applications, for manufacturing biosensors, for performing bacterial display, for whole-cell based biocatalytic applications or for protein production and purification.

15. The method according to claim 14, wherein said polypeptide precursor comprises an antigen, or epitope thereof, or an enzyme, or catalytically active fragment thereof, which will be exposed to the surface of a bacterial cell of the Bacteroidetes phylum comprising said polypeptide precursor.

16. The method according to claim 15, wherein said bacterial cell of the Bacteroidetes phylum is Capnocytophaga canimorsus or Flavobacterium johnsoniae.
